(19) United States
(12) Patent Application Publication
(10) Pub. No.: US 2022/0288121 A1
(43) Pub. Date: Sep. 15, 2022

(54) CELL CRYOPRESERVATION MEDIUM

(71) Applicant: BOARD OF REGENTS, THE UNIVERSITY OF TEXAS SYSTEM, Austin, TX (US)

(72) Inventors: Katy REZVANI, Houston, TX (US); Enli LIU, Houston, TX (US); Elizabeth SHPALL, Houston, TX (US); Rafet BASAR, Houston, TX (US); David MARIN COSTA, Houston, TX (US)

(73) Assignee: Board of Regents, The University of Texas System, Austin, TX (US)

(21) Appl. No.: 17/638,601

(22) PCT Filed: Aug. 25, 2020
(86) PCT No.: PCT/US2020/047774

§ 371 Date: Feb. 25, 2022

Related U.S. Application Data
(60) Provisional application No. 62/893,597, filed on Aug. 29, 2019.
(60) Provisional application No. 63/013,823, filed on Apr. 22, 2020.

(51) Int. Cl. A61K 35/17; A01N 1/02; C12N 5/0783; A61K 35/545; A61K 35/28; A61K 35/15

(52) U.S. Cl. CPC A61K 35/17; A01N 1/0221; C12N 5/0646; A01N 1/0226; A61K 35/545; A61K 35/28; A61K 35/15; C12N 2501/2302; C12N 2501/2315; C12N 2501/2321; C12N 2500/62

(57) ABSTRACT

Provided herein are cryopreservation compositions and methods for cells of any kind, including for cells for adoptive cell therapy that are off-the-shelf cells. The cells for cryopreservation may be expanding NK cells expressing chimeric antigen receptors. In specific cases, the cryopreservation media comprises a cryoprotectant, such as DMSO, glycerol or hydroxyethol starch; serum or a non-serum alternative, such as platelet lysate; and one or more cytokines that are either natural, modified, synthetic, or recombinant.

This application claims priority to U.S. Provisional Patent Application Ser. No. 62/893,597, filed Aug. 29, 2019, and also to U.S. Provisional Patent Application Ser. No. 63/013,823, filed Apr. 22, 2020, both of which are incorporated by reference herein in their entirety.

### BACKGROUND

### 1. Technical Field

The present disclosure relates generally to the fields of cell biology, molecular biology, biochemistry, immunology, and medicine.

### 2. Description of Related Art

Culture of cells, e.g., mammalian cells, for in vitro studies or ex vivo culture for administration to a human or animal is an important tool for the study and treatment of human diseases. Cell culture is widely used for the production of various biologically active products, such as viral vaccines, monoclonal antibodies, polypeptide growth factors, hormones, enzymes and tumor specific antigens. However, many of the media or methods used to culture the cells comprise components that can have negative effects on cell growth and/or maintenance of cells in culture.

In addition, presently several cell banks exist that store cells, for example human placental or umbilical cord stem cells, for future medical use. There are also cell banks that store cells, cultivated in for example bioreactors, for scientific purposes as well as for medical therapies. Common for all cell banks is that the cells are stored by cryopreservation usually in liquid nitrogen. The present disclosure satisfies a need in the art for improved cryopreservation media.

### BRIEF SUMMARY

The present disclosure concerns cell media, including cryopreservation media, that allows the cells to have a more robust proliferation and retention of cell characteristics compared to cells cryopreserved in the absence of the disclosed media and methods. In particular embodiments, the cell cryopreservation media allows for enhanced cell viability of cells that are to be used “off-the-shelf.” Following cryopreservation in the disclosed media, the cells upon thawing may be used immediately or may be further manipulated, such as subject to recombination techniques including transfection, for example. In some cases the cells are cryopreserved a second or subsequent time, whether or not in the disclosed media, and prior to the second or subsequent cryopreservation, the cells may or may not be further manipulated, such as subject to recombination techniques including transfection, for example.

Embodiments of the disclosure provide a cryopreservation medium composition comprising at least one cryoprotectant, a serum (human or animal serum) or a non-serum alternative to serum (not human serum or animal serum), and at least one cytokine and/or at least one growth factor. In some cases, the cryoprotectant is dimethyl sulfoxide (DMSO), glycerin, glycerol, hydroxyethol starch, or a combination thereof. The non-serum alternative may be of any kind, including at least platelet lysate and/or a blood product lysate (for example, human serum albumin). In embodiments of the composition wherein one or more (including two or more) cytokines are utilized, the cytokine may be a natural or a recombinant or a synthetic protein. At least one of the cytokines may be an Food and Drug Administration (FDA)-approved cytokine. Examples of cytokines and growth factors include at least IL-1, IL-2, IL-3, IL-4, IL-6, IL-7, IL-9, IL-10, IL-12, IL-13, IL-15, IL-17, IL-18, IL-21, IL-22, interferon, tumor necrosis factor, stem cell factor, FLT3-ligand, APRIL, thrombopoietin, erythropoietin, or a combination thereof. For serum embodiments, the serum may be an animal-derived serum, such as human serum (including human AB serum) or bovine serum. DMSO and other cryoprotectants, when utilized may comprise 4-10%, 4-6%, 4-8%, 5-10%, 5-8%, 6-10%, 6-8%, 8-10%, and so forth, of the composition. For embodiments wherein serum is employed, the serum may comprise 5-99%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-99%, 10-95%, 10-90%, 10-85%, 10-80%, 10-75%, 10-70%, 10-65%, 10-60%, 10-55%, 10-50%, 10-45%, 10-40%, 10-35%, 10-30%, 10-25%, 10-20%, 10-15%, 20-99%, 20-95%, 20-90%. 20-85%, 20-80%, 20-75%, 20-70%, 20-65%, 20-60%, 20-55%, 20-50%, 20-45%, 20-40%, 20-35%, 20-30%, 20-25%, 30-99%, 30-95%, 30-90%, 30-85%, 30-80%, 30-75%, 30-70%, 30-65%, 30-60%, 30-55%, 30-50%, 30-45%, 30-40%, 30-35%, 40-99%, 40-95%, 40-90%, 40-85%, 40-80%, 40-75%, 40-70%, 40-65%, 40-60%, 40-55%, 40-50%, 40-45%, 50-99%, 50-95%, 50-90%, 50-85%, 50-80%, 50-75%, 50-70%, 50-65%, 50-60%, 50-55%, 60-99%, 60-95%, 60-90%, 60-85%, 60-80%, 60-75%, 60-70%, 60-65%, 70-99%, 70-95%, 70-90%, 70-85%, 70-80%, 70-75%, 80-99%, 80-95%, 80-90%, 80-85%, 90-99%, 90-95%, or 95-99% of the composition. The composition may comprise at least or no more than 10, 15, 20, 25, 30, 35, 40, 45, 50, 55, 60, 65, 70, 75, 80, 85, 90, 91, 92, 93, 94, 95, 96, 97, 98, or 99% of serum. In specific embodiments, the composition comprises platelet lysate that may be at any concentration in the composition, but in certain embodiments the platelet lysate comprises 5-99%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-99%, 10-95%, 10-90%, 10-85%, 10-80%, 10-75%, 10-70%, 10-65%, 10-60%, 10-55%, 10-50%, 10-45%, 10-40%, 10-35%, 10-30%, 10-25%, 10-20%, 10-15%, 20-99%, 20-95%, 20-90%. 20-85%, 20-80%, 20-75%, 20-70%, 20-65%, 20-60%, 20-55%, 20-50%, 20-45%, 20-40%, 20-35%, 20-30%, 20-25%, 30-99%, 30-95%, 30-90%, 30-85%, 30-80%, 30-75%, 30-70%, 30-65%, 30-60%, 30-55%, 30-50%, 30-45%, 30-40%, 30-35%, 40-99%, 40-95%, 40-90%, 40-85%, 40-80%, 40-75%, 40-70%, 40-65%, 40-60%, 40-55%, 40-50%, 40-45%, 50-99%, 50-95%, 50-90%, 50-85%, 50-80%, 50-75%, 50-70%, 50-65%, 50-60%, 50-55%, 60-99%, 60-95%, 60-90%, 60-85%, 60-80%, 60-75%, 60-70%, 60-65%, 70-99%, 70-95%, 70-90%, 70-85%, 70-80%, 70-75%, 80-99%, 80-95%, 80-90%, 80-85%, 90-99%, 90-95%, or 95-99% of the composition. The composition may comprise at least or no more than 10, 15, 20, 25, 30, 35, 40, 45, 50, 55, 60, 65, 70, 75, 80, 85, 90, 91, 92, 93, 94, 95, 96, 97, 98, or 99% of platelet lysate.

The composition may have certain concentrations of components, including cytokines and/or growth factors. In specific cases, any cytokine, including IL-2, IL-21, and/or IL-15, for example, are present in the composition in a particular concentration. The IL-2 may be present at a concentration of 1-5000, 1-1000, 1-500, 1-100, 100-5000, 100-500, 500-5000, 500-1000, or 1000-5000 U/mL, for example. In a specific case, the IL-2 is present at a concentration in the composition of at least or no more than 100, 200, 300, 400, 500, 600, 700, 800, 900, or 1000 U/mL. In specific embodiments, IL-21 is present in the composition at a concentration of 10-3000, 10-2000, 10-1000, 10-500, 10-100, 100-3000, 100-2000, 100-1000, 500-3000, 500-2000, 500-1000, 1000-3000, 1000-2000, or 2000-3000 ng/mL. The IL-21 may be in a concentration in the composition of at least or nor more than 1, 5, 10, 20, 30, 40, 50, 60, 70, 80, 90, 100, 150, 200, 250, 300, 350, 400, 450, 500, 750, 1000, 1250, 1500, 1750, 2000, 2250, 2500, 2750, or 3000 ng/mL. IL-15 may be present in the composition at a concentration of 1-2000, 1-1000, 1-500, 1-100, 100-2000, 100-1000, 100-500, 500-2000, 500-1000, or 1000-2000 ng/mL. IL-15 may be present in the composition at a concentration of at least or no more than 10, 50, 100, 500, 1000, 1500, or 2000 ng/mL.

Compositions as encompassed herein that comprise at least one cryoprotectant, a serum or a non-serum alternative to serum, and at least one cytokine and/or at least one growth factor may further comprise a plurality of immune cells and/or stem cells, each of any kind. In specific embodiments, the cells are NK cells, T cells, B cells, NKT cells derived from mature bone marrow or peripheral blood cells, cell lines such as tumor cell lines (e.g., NK92 or other NK lines), hematopoietic stem cells, induced pluripotent stem cells, MSCs (a population of cells alternatively called “mesenchymal stem cells” and “mesenchymal stromal cells” in the literature), or a mixture thereof, which can be derived from bone marrow, peripheral blood, skin, adipose tissue, or a combination thereof. In embodiments wherein NK cells are utilized, the NK cells may or may not be expanded NK cells. Embodiments of the disclosure also encompass pharmaceutical compositions that comprise any composition of the disclosure and a suitable pharmaceutically acceptable carrier.

Embodiments of the disclosure include methods of producing any composition of the disclosure, comprising the step of subjecting cells to an effective amount of a cryopreservation medium composition. The cells may be immune and/or stem cells. Examples of cells include NK cells, T cells, NKT cells, B cells, NKT cells derived from mature peripheral blood, bone marrow, and/or umbilical cord blood cells, cell lines such as tumor cell lines (e.g., NK92 or other NK lines), stem cells, induced pluripotent stem cells, or MSCs from umbilical cord blood, bone marrow, peripheral blood, adipose tissue, and/or skin. The cells may be expanded NK cells or expanded fractions of any of the cell types encompassed herein.

Embodiments of the disclosure include populations of cells produced according to any method encompassed herein. The population may or may not be comprised in a suitable pharmaceutically acceptable carrier. The cells may be immune cells and/or stem cells of any kind. The cells may be NK cells (whether or not they are expanded), T cells, NKT cells, B cells, NKT cells derived from mature cells, cell lines such as tumor cell lines (e.g., NK92 or other NK lines), stem cells, or induced pluripotent stem cells. Any cells encompassed herein may or may not be expanded. Embodiments of the disclosure include methods of treating an immune-related disorder in a subject comprising administering an effective amount of any thawed population encompassed herein. Examples of immune-related disorders include cancer, an autoimmune disorder, graft versus host disease, an allograft rejection, or an inflammatory condition, including a bacterial, viral or fungal infection. The population may comprise cells that are NK cells, T cells, NKT cells, B cells, NKT cells derived from mature cells, stem cells, induced pluripotent stem cells, or MSCs. Additionally, cancer cells of non-immune origin may be treated with the populations of cells that are NK cells, T cells, NKT cells, B cells, NKT cells derived from mature cells, stem cells, induced pluripotent stem cells, and/or MSCs.

Embodiments of the disclosure include methods of preserving cells that are sensitive to cryopreservation, comprising the step of subjecting cells that are sensitive to cryopreservation to an effective amount of the cryopreservation medium composition of the disclosure. The cells may be NK cells, T cells, NKT cells, B cells, NKT cells derived from mature cells, cell lines such as tumor cell lines (e.g., NK92 or other NK lines), stem cells, induced pluripotent stem cells, or MSCs, for example. The method may or may not further comprise the step of obtaining or providing the cells to be subjected to the cryopreservation medium composition. Following cryopreservation and thawing of the cells, an effective amount of the cells may be delivered to a subject in need thereof, such as one having cancer, autoimmune disorder, graft versus host disease, allograft rejection, or an inflammatory condition, including a bacterial, viral or fungal infection. The cells may be allogeneic or autologous with respect to the subject. Additionally, individuals with organ damage, including at least cardiac, lung, brain and/or kidney, may receive an effective amount of the cryopreserved and thawed cells, including, for example, the MSCs and/or induced pluripotent stem cells for regenerative medicine.

Embodiments of the disclosure include methods of maintaining the viability of a population of cells over at least 50% percent following cryopreservation of the population, comprising the step of subjecting the population to an effective amount of the cryopreservation medium composition encompassed herein and thawing the population, wherein upon thawing the viability of the population is over at least 50%. In some cases, upon thawing of the cells the viability of the population of cells is over at least 55, 60, 65, 70, 75, 80, 85, 90, 91, 92, 93, 94, 95, 96, 97, 98, or 99% following cryopreservation of the population. The cells may be immune and/or stem cells, including NK cells, T cells, NKT cells, B cells, NKT cells derived from mature cells, cell lines such as tumor cell lines (e.g., NK92 or other NK lines), stem cells, induced pluripotent stem cells, or MSCs.

Methods of prolonging the shelf life of a population of cells (for example, immune and/or stem cells) upon cryopreservation of the population are contemplated herein, such as comprising the step of subjecting the population to an effective amount of the cryopreservation medium composition of the disclosure. The shelf life may be prolonged on the order of 1-4, 1-2, 1-3, 2-4, 2-3, or 3-4 weeks, 1-12, 2-12, 3-12, 4-12, 5-12, 6-12, 7-12, 8-12, 9-12, 10-12, or 11-12, months, or 1, 2, 3, 4, 5, 6, 7, 8, 9, 10, 11, 12, 13, 14, 15, 16, 17, 18, 19, 20, or more years compared to shelf life of cryopreserved cells in the absence of use of the cryopreservation medium composition of the disclosure. The cells may or may not be NK cells, T cells, NKT cells, B cells, NKT cells derived from mature cells, cell lines such as tumor cell lines (e.g., NK92 or other NK lines), stem cells, induced pluripotent stem cells, or MSCs. Some methods further comprise the step of obtaining the cells. Following cryopreservation and thawing of the cells, an effective amount of the cells may be delivered to a subject in need thereof. The cells may be allogeneic or autologous with respect to the subject, and the subject may have cancer, autoimmune disorder, graft versus host disease, allograft rejection, or an inflammatory condition, including a bacterial, viral. or fungal infection. The subject may also have vital organ damage in need of regenerative repair.

Embodiments of the disclosure include methods of thawing a population cells that have been cryopreserved with a cryopreservation medium composition encompassed herein, comprising the steps of exposing the population of cells to an effective amount of the cryopreservation medium composition to produce a cryopreserved population; and exposing the cryopreserved population to suitable thawing conditions. The thawing conditions may be standard in the art. For example, one may thaw frozen cells rapidly (<1 minute) in a 37° C. water bath and this may be followed by diluting the thawed cells slowly, optionally using pre-warmed growth medium. In specific cases, thawed cells are plated at high density to optimize recovery.

Certain embodiments of the disclosure concern methods of delivering cells to a target site or tissue in an individual, comprising the step of infusing or administering the cells intravenously, locally, intrathecally, intraperitoneally, subcutaneously an effective amount of cells to the target site or tissue substantially immediately and/or substantially directly following thawing of the cells, wherein the cells were cryopreserved in the cryopreservation medium composition of the disclosure. In specific embodiments, the target site or tissue is cancerous, such as being a solid tumor.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The subject matter of the disclosure may be better understood by reference to one or more of these drawings in combination with the detailed description of specific embodiments presented herein.

FIG. 1 shows viability of CB-NKs cryopreserved in 9 different freezing media formulations containing different combinations of cytokines (n=4).

FIG. 2 demonstrates that NK cells cryopreserved in good manufacturing practice (GMP) freeze media exert inferior cytotoxicity against K562 targets post-thaw compared to fresh NK cells (n=3).

FIG. 3 shows that NK cells cryopreserved in GMP freeze media and cytokines exert similar cytotoxicity against K562 targets post-thaw compared to fresh NK cells (n=3).

FIG. 4 demonstrates that chimeric antigen receptor (CAR)-expressing NK cells cryopreserved in GMP freezing media exert inferior cytotoxicity against Raji targets post-thaw compared to fresh CAR NK cells (n=3).

FIG. 5 shows CAR-expressing NK cells cryopreserved in GMP freeze media and cytokines exert similar cytotoxicity against Raji targets post-thaw compared to fresh CAR NK cells (n=3).

FIG. 6 provides that CAR-expressing NK cells cryopreserved in GMP freezing media and cytokines exert similar cytotoxicity against Raji targets post-thaw compared to fresh CAR NK cells and are superior to CAR NK cells frozen in standard freeze media (n=3).

FIG. 7 shows that CAR-expressing NK cells frozen in novel freeze media+cytokines and infused immediately post-thaw in Raji-engrafted mice exert disease control.

FIG. 8 shows that CAR-expressing NK cells frozen in novel freeze media+cytokines and infused immediately post-thaw in Raji-engrafted mice exert similar disease control as fresh CAR-expressing NK cells, and they are superior to CAR-expressing NK cells frozen in standard GMP freeze media.

FIG. 9 provides an example of a study design for cryopreservation of CAR NK cells using different freezing media conditions.

FIG. 10 shows a variety of freezing conditions having variables such as (1) comparison of RPMI vs PlasmaLyte; (2) comparison of different extracellular cryoprotectants (dextran and human albumin); (3) comparison of cytokines (IL-2/IL-15); and (4) comparison of different intracellular cryoprotectant concentrations (DMSO 5% vs 7.5%).

FIG. 11 provides post-thaw viability and recovery rate for CAR-NK cells frozen in a variety of different media, with a comparison of different concentrations of PlasmaLyte, extracellular cryoprotectant (CPA) (dextran and human albumin); intracellular CPA (DMSO 5% vs 7.5%)+/−cytokines (IL-2/IL-15). Measurements of viability and recovery were performed either immediately post-thaw or 4 hrs after thaw.

FIG. 12 shows expression of CAR on NK cells cryopreserved in media containing different concentrations of PlasmaLyte, extracellular CPA (dextran and human albumin); intracellular CPA (DMSO 5% vs 7.5%)+/−cytokines (IL-2/IL-15) either immediately post-thaw or 4 hrs after thaw.

FIG. 13 shows CD16 expression on NK cells cryopreserved in media containing different concentrations of PlasmaLyte, extracellular CPA (dextran and human albumin); intracellular CPA (DMSO 5% vs 7.5%)+/−cytokines (IL-2/IL-15) either immediately post-thaw or 4 hrs after thaw.

FIG. 14 shows CD56 expression on NK cells cryopreserved in media containing different concentrations of PlasmaLyte, extracellular CPA (dextran and human albumin); intracellular CPA (DMSO 5% vs 7.5%)+/−cytokines (IL-2/IL-15) either immediately post-thaw or 4 hrs after thaw.

FIG. 15 shows the cytotoxicity of CAR NK cells frozen in in freeze media containing different concentrations of PlasmaLyte, extracellular CPA (dextran and human albumin); intracellular CPA (DMSO 5% vs 7.5%)+/−cytokines (IL-2/IL-15) immediately post-thaw against Raji and K562 targets.

FIG. 16 shows the cytotoxicity of CAR NK cells frozen in in freeze media containing different concentrations of PlasmaLyte, extracellular CPA (dextran and human albumin); intracellular CPA (DMSO 5% vs 7.5%)+/−cytokines (IL-2/IL-15) 4 h post-thaw against Raji and K562 targets.

FIG. 17 provides IncuCyte live imaging cytotoxicity assay showing the kinetic of K562 and Raji target killing by CAR NK cells frozen in media containing different concentrations of PlasmaLyte, extracellular CPA (dextran and human albumin); intracellular CPA (DMSO 5% vs 7.5%)+/−cytokines (IL-2/IL-15).

FIG. 18 provides IncuCyte live imaging showing the kinetics of apoptosis post-thaw for CAR NK cells that were frozen with various formulations, thawed and cocultured with Raji cells.

FIG. 19 shows the apoptosis (by Annexin V staining) of CAR NK cells frozen in freeze media containing different concentrations of PlasmaLyte, extracellular CPA (dextran and human albumin); intracellular CPA (DMSO 5% vs 7.5%)+/−cytokines (IL-2/IL-15) 4 h post thaw.

FIG. 20 illustrates testing of the addition of platelet lysate (PLT Lys), PlasmaLyte and AB serum+different cytokine combination (IL-2/IL-15 vs IL-2/IL-21) in the freeze media of GMP grade CAR NK cells and including a comparison of CAR NK cells frozen using the same conditions on after 15 vs 22 days of in vitro expansion.

FIG. 21 illustrates testing of the addition of PLT Lys, PlasmaLyte and AB serum+different cytokine combination (IL-2/IL-15 vs IL-2/IL-21) in the freeze media of GMP grade CAR NK cells, including comparison of CAR NK cells expanded for 15 vs 22 days in vitro and frozen using the same conditions.

FIG. 22 shows the CAR expression post-thaw on CAR NK cells frozen in freeze media containing different concentrations of PLT Lys, PlasmaLyte and AB serum+different cytokine combination (IL-2/IL-15 vs IL-2/IL-21).

FIG. 23 demonstrates CD16 expression post-thaw on CAR NK cells frozen in freeze media containing different concentrations of PLT Lys, PlasmaLyte and AB serum+different cytokine combination (IL-2/IL-15 vs IL-2/IL-21).

FIG. 24 shows CD56 expression post-thaw on CAR NK cells frozen in freeze media containing different concentrations of PLT Lys, PlasmaLyte and AB serum+different cytokine combination (IL-2/IL-15 vs IL-2/IL-21).

FIG. 25 demonstrates an IncuCyte live imaging cytotoxicity assay and the kinetic of K562 and Raji killing by CAR NK cells frozen in freeze media containing PLT Lys, PlasmaLyte and AB serum+different cytokine combination (IL-2/IL-15 vs IL-2/IL-21)-Day 22.

FIG. 26 provides IncuCyte live imaging showing the kinetics of apoptosis post thaw for CAR NK cells that were expanded for 22 days and frozen with various formulations, thawed and cocultured with Raji cells.

FIG. 27 shows a study for titration of components of the extracellular cryoprotectant to minimize ice recrystallization: PLT Lys (25% vs 50%); dextran (25% vs 50%; in NACL or dextrose); human albumin (20% vs 45% vs 70%). All conditions tested with a combination of two cytokines (IL-2/IL-15).

FIG. 28 shows the viability results for CAR NK cells frozen in media containing different concentrations of the extracellular cryoprotectant to minimize ice recrystallization: PLT Lys (25% vs 50%); dextran (25% vs 50%; in NACL or dextrose); human albumin (20% vs 45 vs 70%). All conditions tested with a combination of two cytokines (IL-2/IL-15). The viability was tested immediately post-thaw.

FIG. 29 provides the percentage of Annexin expressing NK cells as a measure of apoptosis post-thaw for CAR NK cells that were frozen with media containing different components of the extracellular cryoprotectant to minimize ice recrystallization: PLT Lys (25% vs 50%); dextran (25% vs 50%; in NACL or dextrose); human albumin (20% vs 45 vs 70%). All conditions tested with a combination of two cytokines (IL-2/IL-15).

FIG. 30 demonstrates an IncuCyte live imaging cytotoxicity assay and the kinetic of K562 and Raji killing by CAR NK cells frozen in media containing different concentrations of PLT Lys (25% vs 50%), dextran (25% vs 50%; in NACL or in dextrose) and human albumin (20% vs 45 vs 70%).

FIG. 31 shows IncuCyte live imaging showing the kinetics of apoptosis post thaw for CAR NK cells that were expanded for 22 days and frozen with various formulations, thawed and cocultured with Raji cells.

FIG. 32 provides a study for titration of components of the extracellular cryoprotectant to minimize ice recrystallization: PLT Lys (25% vs 50%); dextran (25% vs 50%; in NACL or in dextrose); human albumin (20% vs 45% vs 70%). All conditions tested with a combination of two cytokines (IL-2/IL-15).

FIG. 33 shows determination of the viability and recovery of CAR NK cells frozen in cryopreservation media containing PLT Lys (25% vs 50%); dextran (25% vs 50%; in NACL or dextrose); human albumin (20% vs 45% vs 70%). All conditions tested with a combination of two cytokines (IL-2/IL-15)

FIG. 34 provides examination of CAR expression on CAR NK cells frozen in: PLT Lys (25% vs 50%); dextran (25% vs 50%; in NACL or in dextrose); human albumin (20% vs 45% vs 70%). All conditions tested with a combination of two cytokines (IL-2/IL-15).

FIG. 35 provides an examination of cytotoxicity of CAR NK cells expanded for 15 days and frozen in media containing: PLT Lys (25% vs 50%); dextran (25% vs 50%; in NACL or in dextrose); human albumin (20% vs 45% vs 70%). All conditions were tested with a combination of two cytokines (IL-12/IL-15). CAR NK cell cytotoxicity was measured by 51 chromium release assay immediately post-thaw.

FIG. 36 shows an IncuCyte live imaging cytotoxicity assay and the kinetics of K562 and Raji killing by CAR NK cells expanded for 15 days, cryopreserved and then tested immediately post-thaw

FIG. 37 illustrates a plan to test the in vivo antitumor activity of GMP grade CAR NK cells frozen in media containing PLT Lys, PlasmaLyte and AB serum+different cytokine combination (IL-2/IL-15 vs IL-2/IL-21), followed by comparing cells that were expanded for either 15 days or 22 days and frozen using the same cryopreservation conditions.

FIG. 38 illustrates a titration of components of the extracellular cryoprotectant to minimize ice recrystallization. The freeze media include: PLT Lys (25% vs 50%); dextran (25% vs 50%; in NACL or in dextrose); human albumin (20% vs 45% vs 70%). All conditions tested with a combination of two cytokines (IL-12/IL-15).

FIG. 39 shows a titration of components of the extracellular cryoprotectant to minimize ice recrystallization. The freeze media include: PLT Lys (25% vs 50%); dextran (25% vs 50%; in NACL or in dextrose); human albumin (20% vs 45 vs 70%). All conditions tested without cytokines, with one cytokine only (IL-2 or IL-15) or with a combination of two cytokines (IL-12/IL-15).

FIG. 40 shows a plan to test of the addition of PLT Lys, PlasmaLyte and AB serum+different cytokine combination (IL-2/IL-15 vs IL-2/IL-21) in the freeze media. GMP grade CAR NK cells were expanded for 15 days vs 22 days and cryopreserved using the different freezing media.

FIG. 41 provides a table with the description of the freezing media used to cryopreserve GMP-grade CAR NK cells that were expanded for 22 days and used for the in vivo mouse study.

FIG. 42 shows survival of mice infused with Raji tumor cells and treated with CAR NK cryopreserved in various freezing media formulations. One cohort received fresh CD19 CAR NK cells (positive control), 11 cohorts received frozen CAR NK cells that were cryopreserved in different freeze media and infused immediately post-thaw. One cohort did not receive CARNK cells (negative control). Mice that received CAR NK cells had a statistically significant superior survival compared to mice that remained untreated irrespectively of the freeze media used to cryopreserve the CAR NK cells, however for cohorts #6, #8 and #11, the survival was clearly inferior to the survival of mice that received fresh CAR NK cells. Mice treated in cohorts #1 (HR=0.811, p=0.78), #2 (HR=0.6, p=0.49), #3 (HR=0.916, p=0.90), #4 (HR=0.859, p=0.83) and #7 (HR=0.883, p=0.87) had superior survival, although it was not statistically significant compared to mice treated with the fresh CAR NK cell product.

FIG. 43 demonstrates anti-tumor activity of frozen CAR NK cells compared to fresh CAR NK cells in Raji mouse model as assessed by BLI.

FIGS. 44-45 show the average radiance for mice treated with CAR NK cells frozen using the different conditions listed in FIG. 41 compared to mice treated with fresh CAR NK cells or no treatment as positive and negative controls, respectively.

### DESCRIPTION OF ILLUSTRATIVE EMBODIMENTS

One limitation of using certain cryopreserved cells for clinical therapy is because of their small numbers and their poor survival post thaw. The present disclosure has addressed both of these limitations by using GMP-compliant strategy for the ex vivo expansion of cells following cryopreservation. Embodiments of the present methods resulted in a much greater survival rate following thaw. In specific embodiments, any method disclosed herein indicates that this strategy could also be applied to cells without prior expansion.

Accordingly, certain embodiments of the present disclosure provide methods and compositions concerning the preservation, such as for storage, of clinical-grade cells, including those intended for cellular and immunotherapy. Growing and molding clinically relevant numbers of cells for infusion into patients while meeting time constraints are extremely challenging even in the best of circumstances. The disclosed methods and compositions detail the technical processes of cellular preservation prior to use of any kind.

In particular embodiments, further provided herein is a freezing media formulation for the preservation of any type of mammalian cells, including immune cells and/or stem cells. The immune cells may be of any kind, including NK cells, T cells, B cells, NKT cells, stem cells, induced pluripotent stem cells (iPSCs) or any cell derived from iPSCs, MSCs, differentiated or committed cells from any organ, any fibroblasts. In any case, the mammalian cells may be utilized for adoptive cell therapy. In specific cases, the cells are CAR NK cells. In specific embodiments, the freezing media may comprise a cryoprotectant such as (but not limited to) dimethyl sulfoxide (DMSO), glycerin, glycerol, hydroxyethol starch, or a combination thereof, serum from human, bovine or other animal source, or a serum alternative such as (but not limited) to platelet lysate, one or more cytokines or growth factors included but not limited to IL-1, IL-2, IL-3, IL-4, IL-6, IL-7, IL-9, IL-10, IL-12, IL-13, IL-15, IL-17, IL-18, IL-21, IL-22, interferon, tumor necrosis factor, stem cell factor, FLT3-ligand, APRIL, or a combination thereof. Serum may be utilized as a source of growth factors, adhesion factors, hormones, lipids and/or minerals and/or in certain cases is used to regulate cell membrane permeability and serves as a carrier for lipids, enzymes, micronutrients, and trace elements into the cell. The freezing media allows for the successful freezing of individual doses of cells with improved viability and functionality. The cells may be thawed and infused into patients per demand. Thus, the frozen cells provide herein are an “off-the-shelf” cell therapy that can be thawed and infused into patients with no delay needed for production.

The media allows for adoptive cell therapy cells to be stored as banks of cells for any purpose, including immunotherapy, without the need to recruit donors for cell collection, although this approach may also be used for the cryopreservation of autologous patient-directed products, as well.

### I. Definitions

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. The terms “about”, “substantially” and “approximately” mean, in general, the stated value plus or minus 5%. As used herein “another” may mean at least a second or more.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

Throughout this application, the term “about” is used to indicate that a value includes the inherent variation of error for the device, the method being employed to determine the value, or the variation that exists among the study subjects.

An “immune disorder,” “immune-related disorder,” or “immune-mediated disorder” refers to a disorder in which the immune response plays a key role in the development or progression of the disease. Immune-mediated disorders include autoimmune disorders, allograft rejection, graft versus host disease and inflammatory and allergic conditions.

An “immune response” is a response of a cell of the immune system, such as a B cell, or a T cell, or innate immune cell to a stimulus. In one embodiment, the response is specific for a particular antigen (an “antigen-specific response”).

An “autoimmune disease” refers to a disease in which the immune system produces an immune response (for example, a B-cell or a T-cell response) against an antigen that is part of the normal host (that is, an autoantigen), with consequent injury to tissues. An autoantigen may be derived from a host cell, or may be derived from a commensal organism such as the microorganisms (known as commensal organisms) that normally colonize mucosal surfaces.

“Treating” or treatment of a disease or condition refers to executing a protocol, which may include administering one or more drugs or cellular therapy products to a patient, in an effort to alleviate signs or symptoms of the disease. Desirable effects of treatment include decreasing the rate of disease progression, ameliorating or palliating the disease state, and remission or improved prognosis. Alleviation can occur prior to signs or symptoms of the disease or condition appearing, as well as after their appearance. Thus, “treating” or “treatment” may include “preventing” or “prevention” of disease or undesirable condition. In addition, “treating” or “treatment” does not require complete alleviation of signs or symptoms, does not require a cure, and specifically includes protocols that have only a marginal effect on the patient.

The term “therapeutic benefit” or “therapeutically effective” as used throughout this application refers to anything that promotes or enhances the well-being of the subject with respect to the medical treatment of this condition. This includes, but is not limited to, a reduction in the frequency or severity of the signs or symptoms of a disease. For example, treatment of cancer may involve, for example, complete eradication of the tumor, a reduction in the size of a tumor, a reduction in the invasiveness of a tumor, reduction in the growth rate of the cancer, or prevention of metastasis. Treatment of cancer may also refer to prolonging survival of a subject with cancer.

“Subject” and “patient” and “individual” may be interchangeable and may refer to either a human or non-human, such as primates, mammals, and vertebrates. In particular embodiments, the subject is a human. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as one or more infectious diseases, one or more genetic disorders, one or more cancers, or any combination thereof. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (e.g., children) and infants and includes in utero individuals. A subject may or may not have a need for medical treatment; an individual may voluntarily or involuntarily be part of experimentation whether clinical or in support of basic science studies.

The phrases “pharmaceutical or pharmacologically acceptable” refers to molecular entities and compositions that do not produce an adverse, allergic, or other untoward reaction when administered to an animal, such as a human, as appropriate. The preparation of a pharmaceutical composition comprising an antibody or additional active ingredient will be known to those of skill in the art in light of the present disclosure. Moreover, for animal (e.g., human) administration, it will be understood that preparations should meet sterility, pyrogenicity, general safety, and purity standards as required by FDA Office of Biological Standards.

As used herein, “pharmaceutically acceptable carrier” includes any and all aqueous solvents (e.g., water, alcoholic/aqueous solutions, saline solutions, parenteral vehicles, such as sodium chloride, Ringer's dextrose, etc.), non-aqueous solvents (e.g., propylene glycol, polyethylene glycol, vegetable oil, and injectable organic esters, such as ethyloleate), dispersion media, coatings, surfactants, antioxidants, preservatives (e.g., antibacterial or antifungal agents, anti-oxidants, chelating agents, and inert gases), isotonic agents, absorption delaying agents, salts, drugs, drug stabilizers, gels, binders, excipients, disintegration agents, lubricants, sweetening agents, flavoring agents, dyes, fluid and nutrient replenishers, such like materials and combinations thereof, as would be known to one of ordinary skill in the art. The pH and exact concentration of the various components in a pharmaceutical composition are adjusted according to well-known parameters.

The term “antigen presenting cells (APCs)” refers to a class of cells capable of presenting one or more antigens in the form of a peptide-MHC complex recognizable by specific effector cells of the immune system, and thereby inducing an effective cellular immune response against the antigen or antigens being presented. The term “APC” encompasses intact whole cells such as macrophages, B-cells, endothelial cells, activated T-cells, dendritic cells, cell lines (such as K562), or molecules, naturally occurring or synthetic, capable of presenting antigen, such as purified MHC Class I molecules complexed to β2-microglobulin.

### II. Cryopreservation Medium and Use Thereof

Cells of any kind may be preserved in the cryopreservation media of the disclosure. The cells may be mammalian, in certain embodiments, and in specific cases they are mammalian cells to be utilized for research and/or therapy. The cells may be immune cells, in specific cases, including immune cells to be utilized for adoptive cell therapy. Such cells may or may not be NK cells, T cells, NKT cells, B cells, stem cells, induced pluripotent stem cells (iPSCs) or any cell derived from iPSCs, MSCs, differentiated or committed cells from any organ, any fibroblasts, and so forth. The cells may be obtained from an individual, cryopreserved using media encompassed herein, and then thawed and used for the individual and/or for another one or more other individuals. The cells may be obtained from an individual, manipulated to comprise one or more characteristics in addition to those without the manipulation, cryopreserved using media encompassed herein, and used for the individual and/or for another one or more other individuals.

A first plurality of cells from one collection of cells may be cryopreserved in one particular cryopreservation media encompassed herein, while a second plurality of cells from the same collection of cells may be cryopreserved in a different cryopreservation media also encompassed herein. Such a practice may or may not be employed depending on the application of the cells, the number and/or viability of the cells, and so forth.

In particular embodiments, cells are preserved in the cryopreservation media encompassed herein substantially immediately following collecting them from one or more individuals. In other embodiments, cells are cryopreserved following culture or expansion. Following expansion, the cells (such as immune cells) may be immediately manipulated for a later purpose (such as infused), or they may be stored through cryopreservation. In certain aspects, the cells may be propagated for days, weeks, or months ex vivo as a bulk population within about 1, 2, 3, 4, 5, 6, 7, 8, 9, 10, 11, 12, 13, 14, or more days.

In particular embodiments, the cryopreservation media may comprise dimethyl sulfoxide (DMSO); serum (including human serum); and one or more cytokines of any kind. In specific embodiments, any one or more components of the cryopreservation media are natural proteins, which may also be referred to as endogenous or recombinant proteins. In specific cases the endogenous proteins are the one or more cytokines. The cryopreservation media may also comprise one or more FDA-approved agents, and the one or more FDA-approved agents may be the one or more cytokines, in certain cases.

Particular embodiments of the disclosure include cryopreservation media composition that comprises, consists of, or consists essentially of at least one cryoprotectant, at least one serum (or non-serum alternative to serum), and at least one cytokine and/or at least one growth factor. Examples of cryoprotectants include dimethyl sulfoxide (DMSO), glycerin, glycerol, hydroxyethol starch, or a combination thereof. For the composition, the non-serum alternative may comprise platelet lysate and/or a blood product lysate and/or human serum albumin and/or animal serum albumin. The human serum may be human AB serum. Any cytokine may be a natural protein, a recombinant protein, a synthetic protein, or a mixture thereof, including at least one cytokine being a Food and Drug Administration (FDA)-approved cytokine. In specific cases, the composition comprises two or more cytokines. Merely as examples, at least one cytokine is IL-1, IL-2, IL-3, IL-4, IL-6, IL-7, IL-9, IL-10, IL-12, IL-13, IL-15, IL-17, IL-18, IL-21, IL-22, interferon, tumor necrosis factor, stem cell factor, FLT3-ligand, APRIL, or a combination thereof.

In particular cases, the one or more cytokines include IL-2, IL-15, IL-12, IL-18, and/or IL-21. The cells may be suspended in GMP cryopreservation medium comprising DMSO (e.g., 1-10%, such as 1, 2, 3, 4, 5, 6, 7, 8, 9 or 10%, particularly 5%), 95% Human AB Serum (e.g., 90-99%, such as 91, 92, 93, 94, 95, 96, 97, 98, or 99%, particularly 95%), Platelet lysate (e.g., 90-99%, such as 91, 92, 93, 94, 95, 96, 97, 98, or 99%, particularly 95%), IL-2 (e.g., 50-500 U/mL, such as 100, 200, 300, 400, 500, 1000, or 5000 U/mL, particularly 400 U/mL), IL-15 (5-500 ng/ml) and/or IL-21 (e.g., 1-500 ng/mL, such as 10, 20, 30, 40, 50, 100, or 500 ng/mL, particularly 20 ng/mL). In particular cases, the cells are frozen in liquid nitrogen using a rate controlled method.

In particular embodiments, the cryoprotectant comprises a particular amount of the composition; in specific aspects, the cryoprotectant comprises 4-6% of the composition or 5-10% of the composition; in specific cases, the cryoprotectant comprises 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10% of the composition. The serum may comprise a particular amount of the composition, such as comprising 5-99, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-90, 10-85, 10-80, 10-75, 10-70, 10-65, 10-60, 10-55, 10-50, 10-45, 10-40, 10-35, 10-30, 10-25, 10-20, 10-15, 25-99, 25-90, 25-85, 25-08, 25-75, 25-70, 25-65, 25-60, 25-55, 25-50, 25-45, 25-40, 25-35, 25-30, 50-99, 50-90, 50-85, 50-80, 50-75, 50-70, 40-65, 50-60, or 50-55% of the composition. The plateley lysate may comprise a certain amount of the composition, such as 5-99, 5-90, 5-85, 5-80, 5-75, 5-70, 5-65, 5-60, 5-55, 5-50, 5-45, 5-40, 5-35, 5-30, 5-25, 5-20, 5-15, 5-10, 10-99, 10-90, 10-85, 10-80, 10-75, 10-70, 10-65, 10-60, 10-55, 10-50, 10-45, 10-40, 10-35, 10-30, 10-25, 10-20, 10-15, 25-99, 25-90, 25-85, 25-08, 25-75, 25-70, 25-65, 25-60, 25-55, 25-50, 25-45, 25-40, 25-35, 25-30, 50-99, 50-90, 50-85, 50-80, 50-75, 50-70, 40-65, 50-60, or 50-55% of the composition. In specific aspects, the platelet lysate comprises 95% of the composition.

In embodiments wherein the composition comprises IL-2, it may be present at a concentration of 1-5000, 1-4000, 1-3000, 1-2000, 10-1000, 100-5000, 100-4000, 100-3000, 100-1000,100-1000, 100-500,500-5000, 500-4000,500-3000, 500-2000, 500-1000, 1000-5000, 1000-4000, 1000-3000, 1000-2000, or 2000-5000 U/mL, including specifically at 100, 200, 300, 400, or 500 U/mL. In embodiments wherein the composition comprises IL-21, it may be present at a concentration of 10-3000, 10-2500, 10-2000, 10-1000, 10-500, 100-3000, 100-2000, 100-1000, 500-3000, 500-2000, 500-1000, or 1000-3000 ng/mL, including specifically being present at a concentration of 10, 15, 20, or 25 ng/mL. In specific cases, the IL-15 is present in the composition at a concentration of 10-2000, 10-1000, 10-500, 100-2000, 100-1000, 100-500, 500-2000, 500-1000, or 1000-2000 ng/mL.

For cryopreservation, as one example, the cells (such as NK cells for adoptive therapy, including cord blood NK cells) may be suspended in a GMP cryopreservation medium comprising, for example, 5% DMSO, 95% Human AB Serum, 400 units IL-2/ml, and 20 ng IL-21/ml. They may be frozen using liquid nitrogen, a non-liquid nitrogen freezer, via dump freezing, a rate-controlled freezing method, and/or a non-rate controlled freezing method, for example.

In specific embodiments, the medium comprises glucose, a pH indicator, one or more salts, one or more amino acids, and one or more vitamins. Examples of pH indicators include at least phenol red, bromophenol blue, methyl orange, bromocresol purple, Congo red, and so forth. Examples of salts include at least sodium chloride, sodium bicarbonate, disodium phosphate, potassium chloride, magnesium sulfate, calcium nitrate, or a combination thereof. Examples of amino acids include glutamine, arginine, asparagine, cysteine, leucine, isoleucine, lysine, serine, aspartic acid, glutamic acid, hydroxyproline, proline, threonine, tyrosine, valine, histidine, methionine, phenylalanine, glycine, tryptophan, reduced glutathione, or a combination thereof. In some embodiments, one or more amino acids are greater in amount in the media than one or more other amino acids, whereas one or more other amino acids may be in the same amount in the media. For example, glutamine may or may not be greatest in amount in the media, followed by arginine. Asparagine, cysteine, leucine, isoleucine, or a combination thereof may or may not be substantially the same amount in the media. Aspartic acid, glutamic acid, hydroxyproline, proline, threonine, tyrosine, valine, or a combination thereof may or may not be substantially the same amount in the media. Histidine, methionine, phenylalanine, or a combination thereof may or may not be substantially the same amount in the media. One or more specific vitamins may be present in the media, including i-inositol; choline chloride; para-aminobenzoic acid, folic acid, nicotinamide, pyridoxine hydrochloride, thiamine hydrochloride; calcium pantothenate; biotin; riboflavin; cyanocobalamin; or a combination thereof may be present in the media. The vitamins may or may not be present in the media at specific amounts. For example, i-inositol may be present in the greatest amount, followed by choline chloride. Certain vitamins may be substantially equal in the media, including para-aminobenzoic acid, folic acid, nicotinamide, pyridoxine hydrochloride, thiamine hydrochloride, or a combination thereof, in some cases. Biotin and riboflavin may or may not be essentially equal in amount in the media. Cyanocobalamin may or may not be present as the least amount of any vitamin in the media.

In some embodiments, the cells may be cultured in a media that is substantially similar or identical to RPMI 1640 medium, also known as RPMI medium, that is a growth medium developed by Moore et al. (Moore G E, Gerner R E, Franklin H A (1967). “Culture of normal human leukocytes”. JAMA. 199 (8): 519-524) at Roswell Park Memorial Institute.

In a specific example, one liter of RPMI 1640 contains or comprises the following:

Glucose (2 g); pH indicator (phenol red, 5 mg); Salts (6 g sodium chloride, 2 g sodium bicarbonate, 1.512 g disodium phosphate, 400 mg potassium chloride, 100 mg magnesium sulfate, and 100 mg calcium nitrate); Amino acids (300 mg glutamine; 200 mg arginine; 50 mg each asparagine, cystine, leucine, and isoleucine; 40 mg lysine hydrochloride; 30 mg serine; 20 mg each aspartic acid, glutamic acid, hydroxyproline, proline, threonine, tyrosine, and valine; 15 mg each histidine, methionine, and phenylalanine; 10 mg glycine; 5 mg tryptophan; and 1 mg reduced glutathione); and Vitamins (35 mg i-inositol; 3 mg choline chloride; 1 mg each para-aminobenzoic acid, folic acid, nicotinamide, pyridoxine hydrochloride, and thiamine hydrochloride; 0.25 mg calcium pantothenate; 0.2 mg each biotin and riboflavin; and 0.005 mg cyanocobalamin).

In specific embodiments, the composition comprises: a) one or more of platelet lysate, PlasmaLyte, and Roswell Park Memorial Institute (RPMI) media; (b) one or more of dextran that can be formulated in dextrose or in saline (for example), albumin, and DMSO; and (c) one or more of IL-2, IL-15, and IL-21. In specific embodiments, any composition comprises platelet lysate between 50% and 90% of the composition, including about 50% of the composition or about 90% of the composition. In cases wherein PlasmaLyte is utilized, it may be between about 32.5% and 70% of the composition, including at about 32.5%, 35%, 50%, or 70% of the composition. The RPMI may be between 32.5% and 50% of the composition, including at about 32.5%, 35%, or 50% of the composition. In cases wherein dextran is utilized, the dextran may be about 25-40% of the composition, including at about 25% or about 40% of the composition. In cases wherein albumin is utilized, it may be about 1-99% of the composition, including at about 20% of the composition. In cases wherein DMSO is utilized, it may be about 5-7.5% of the composition, including specifically at about 5% or 7.5% of the composition.

### III. Cells for Cryopreservation

Cells to be cryopreserved may be of any kind including prokaryotic or eukaryotic, but in specific embodiments the cells are mammalian cells. In specific embodiments, the mammalian cells are obtained from one or more individuals. The mammalian cells may be utilized for research or therapeutic purposes of any kind. In specific embodiments, the cells are immune cells of any kind, including NK cells, T cells, NK T cells, PBMCs, antigen presenting cells (APCs), B cells, mononuclear cells, dendritic cells, monocytes, neutrophils, induced pluripotent stem cells (iPSCs) or any cell derived from iPSCs, MSCs, differentiated or committed cells from any organ, any fibroblasts, and so forth. The cells may or may not be stem cells, in some examples.

The cells in particular embodiments are modified prior to and/or after cryopreservation. For example, they may be transfected or transduced with a vector or electroporated with a plasmid that encodes a particular gene product, such as a gene product that imparts a therapeutic activity to the cells. In specific embodiments, the cells are transfected or transduced or electroporated with one or more antigen receptors, including T cell receptors or chimeric antigen receptors (CARs), cytokines, homing receptors or any other genes. In specific cases, the cells are CAR-expressing immune cells, such as CAR-expressing NK cells.

In certain embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art. Specifically, the NK cells may be isolated from cord blood (CB), peripheral blood (PB), bone marrow, or stem cells. In particular embodiments, the immune cells are isolated from pooled CB. The CB may be pooled from 2, 3, 4, 5, 6, 7, 8, 10, or more units. The immune cells may be autologous or allogeneic. The isolated NK cells may be completely matched, completely mismatched, haplotype matched (half matched) or more than haplotype but less than completely matched with the subject to be administered the cell therapy. NK cells can be detected by specific surface markers, such as CD16 and CD56 in humans.

In certain aspects, the starting population of NK cells is obtained by isolating mononuclear cells using ficoll density gradient centrifugation. The cell culture may be depleted of any cells expressing CD3, CD14, and/or CD19 cells and may be characterized to determine the percentage of CD56⁺/CD3⁻ cells or NK cells. They may also be subjected to positive selection with CD56 or other specific NK cell antibodies, in certain procedures.

The cells may be expanded in the presence of APCs, such as universal APCs. The expansion may be for about 2-30 days, or longer, such as 3-20 days, particularly 12-16 days, such as 12, 13, 14, 15, 16, 17, 18, or 19 days, specifically about 14 days. The NK cells and APCS may be present at a ratio of about 3:1-1:3, such as 2:1, 1:1, 1:2, specifically about 1:2. The expansion culture may further comprise cytokines to promote expansion, such as IL-2, IL-2, IL-15, IL-21, and/or IL-18. The cytokines may be present at a concentration of about 10-500 U/mL, such as 100-300 U/mL, particularly about 200 U/mL. The cytokines may be replenished in the expansion culture, such as every 2-3 days. The APCs may be added to the culture at least a second time, such as after CAR transduction. In particular embodiments, the cytokines are present in the cryopreservation medium at a level that avoids providing a therapeutic effect to the individual upon receipt of the cells, for example if and when the medium is included with the cells upon administering them to a subject. The cells may be comprised in at least some of the cryopreservation medium either because of residual medium upon preparation of the cells for administering, or the cells may be comprised in at least some of the cryopreservation medium by intended design. Following thawing of the cells, the cells may or may not be washed prior to administering to a subject.

In one embodiment, the starting population of cells are MNCs isolated from a single CB unit by ficoll density gradient. The cells can then be washed and depleted of the CD3, CD14 and CD19 positive cells, such as by using the CliniMACS immunomagnetic beads (Miltenyi Biotec). The unlabeled, enriched CB-NK cells can be collected, washed with CliniMACS buffer, counted, and combined with irradiated (e.g., 100 Gy) APCs, such as in a 1:2 ratio. The cell mixture (e.g., 1×10⁶ cells/mL) may be transferred to cell culture flasks containing NK Complete Medium (e.g., 90% Stem Cell Growth Medium, 10% FBS, 2 mM L-glutamine) and IL-2, such as 50-500, such as 100-300, such as 200 U/mL. The cells can be incubated at 37° C. in 5% CO₂. On Day 3, a media change may be performed by collecting the cells by centrifugation and resuspending them in NK Complete Medium (e.g., 1×10⁶ cells/mL) containing IL-2, such as 50-500, such as 100-300, such as 200 U/mL. The cells may be incubated at 37° C. in 5% CO₂. On Day 5, the number of wells needed for Retronectin transduction can be determined by the number of CB-NK cells in culture. The RetroNectin solution may be plated to wells of 24-well culture plates. The plates can be sealed and stored in a 4° C. refrigerator.

On Day 6, a 2^{nd} NK selection as described on Day 0 can be performed prior to transduction of the CB-NK cells. The cells can be washed with CliniMACS buffer, centrifuged and resuspended in NK Complete Medium at 0.5×10⁶/mL with IL-2, such as 100-1000, particularly 600 U/mL. The RetroNectin plates can then be washed with NK complete medium and incubated at 37° C. until use. The NK complete medium in each well can be replaced with retroviral supernatant, followed by centrifugation of plates at 32° C. The retroviral supernatant may then be aspirated and replaced with fresh retroviral supernatant. The CB-NK cell suspension containing 0.5×10⁶ cells and IL-2, 600 U/mL, may be added to each well, and the plates may be centrifuged. The plates can then be incubated at 37° C. with 5% CO₂. On Day 9, the CAR transduced CB-NK cells can be removed from the transduction plates, collected by centrifugation and stimulated with irradiated (e.g, 100 Gy) aAPCs, such as in a ratio of 1:2, in NK Complete Medium with IL-2, 200 U/mL. The cell culture flasks were incubated at 37° C. with 5% CO₂. On Day 12, media change may be performed. On Day 14, the cells can be collected by centrifugation, the supernatant may be aspirated and the cells can be resuspended in fresh NK Complete Medium containing IL-2, 200 U/mL. The cell culture flasks are incubated at 37° C. with 5% CO₂. If more than 1×10⁵ CD3⁺ cells/kg are present, a magnetic immunodepletion of CD3⁺ cells may be performed using CliniCliniMACS CD3 Reagent. On Day 15, the cells are harvested and the final product is prepared for infusion or cryopreservation.

Expanded NK cells can secrete type I cytokines, such as interferon-γ, tumor necrosis factor-α and granulocyte-macrophage colony-stimulating factor (GM-CSF), which activate both innate and adaptive immune cells as well as other cytokines and chemokines. The measurement of these cytokines can be used to determine the activation status of NK cells. In addition, other methods known in the art for determination of NK cell activation may be used for characterization of the NK cells of the present disclosure.

In specific embodiments, the cells are manipulated to express one or more engineered antigen receptors (including one or more chimeric antigen receptors and/or one or more engineered TCRs); one or more cytokines; one or more suicide genes; CD47; HLA-G; HLA-E; or a combination thereof.

A. Chimeric Antigen Receptors

In some embodiments, the cells to be cryopreserved are manipulated to express one or more CARs, either before cryopreservation and/or after cryopreservation. In specific embodiments, the CAR comprises: a) at least one intracellular signaling domain, b) a transmembrane domain, and c) an extracellular domain comprising at least one antigen binding region. Optionally the CAR may comprise one or more costimulatory domains.

In some embodiments, the engineered antigen receptors include CARs, including activating or stimulatory CARs, costimulatory CARs (see WO 2014/055668), and/or inhibitory CARs (iCARs, see Fedorov et al., 2013). The CARs generally include an extracellular antigen (or ligand) binding domain linked to one or more intracellular signaling components, in some aspects via linkers and/or transmembrane domain(s). Such molecules typically mimic or approximate a signal through a natural antigen receptor, a signal through such a receptor in combination with a costimulatory receptor, and/or a signal through a costimulatory receptor alone.

Certain embodiments of the present disclosure concern the use of nucleic acids, including nucleic acids encoding an antigen-specific CAR polypeptide, including a CAR that has been humanized to reduce immunogenicity (hCAR), comprising an intracellular signaling domain, a transmembrane domain, and an extracellular domain comprising one or more signaling motifs. In certain embodiments, the CAR may recognize an epitope comprising the shared space between one or more antigens. In certain embodiments, the binding region can comprise complementary determining regions of a monoclonal antibody, variable regions of a monoclonal antibody, and/or antigen binding fragments thereof. In another embodiment, that specificity is derived from a peptide (e.g., cytokine) that binds to a receptor.

It is contemplated that the human CAR nucleic acids may be human genes used to enhance cellular immunotherapy for human patients. In a specific embodiment, the invention includes a full-length CAR cDNA or coding region. The antigen binding regions or domain can comprise a fragment of the V_{H} and V_{L} chains of a single-chain variable fragment (scFv) derived from a particular human monoclonal antibody, such as those described in U.S. Pat. No. 7,109,304, incorporated herein by reference. The fragment can also be any number of different antigen binding domains of a human antigen-specific antibody. In a more specific embodiment, the fragment is an antigen-specific scFv encoded by a sequence that is optimized for human codon usage for expression in human cells. The CAR may be bi-specific for two non-identical antigenic targets or tri-specific for three non-identical antigenic targets, and so forth.

The arrangement could be multimeric, such as a diabody or multimers. The multimers are most likely formed by cross pairing of the variable portion of the light and heavy chains into a diabody. The hinge portion of the construct can have multiple alternatives from being totally deleted, to having the first cysteine maintained, to a proline rather than a serine substitution, to being truncated up to the first cysteine. The Fc portion can be deleted. Any protein that is stable and/or dimerizes can serve this purpose. One could use just one of the Fc domains, e.g., either the CH2 or CH3 domain from human immunoglobulin. One could also use the hinge, CH2 and CH3 region of a human immunoglobulin that has been modified to improve dimerization. One could also use just the hinge portion of an immunoglobulin. One could also use portions of CD8alpha.

In some embodiments, the CAR nucleic acid comprises a sequence encoding other costimulatory receptors, such as a transmembrane domain and a modified CD28 intracellular signaling domain. Other costimulatory receptors include, but are not limited to one or more of CD28, CD27, OX-40 (CD134), DAP10, DAP12, CD40 ligand, and 4-1BB (CD137). In addition to a primary signal initiated by CD3ζ, an additional signal provided by a human costimulatory receptor inserted in a human CAR is important for full activation of NK cells and could help improve in vivo persistence and the therapeutic success of the adoptive immunotherapy.

In some embodiments, CAR is constructed with a specificity for a particular antigen (or marker or ligand), such as an antigen expressed in a particular cell type to be targeted by adoptive therapy, e.g., a cancer marker, and/or an antigen intended to induce a dampening response, such as an antigen expressed on a normal or non-diseased cell type. Thus, the CAR typically includes in its extracellular portion one or more antigen binding molecules, such as one or more antigen-binding fragment, domain, or portion, or one or more antibody variable domains, and/or antibody molecules. In some embodiments, the CAR includes an antigen-binding portion or portions of an antibody molecule, such as a single-chain antibody fragment (scFv) derived from the variable heavy (VH) and variable light (VL) chains of a monoclonal antibody (mAb).

In certain embodiments of the chimeric antigen receptor, the antigen-specific portion of the receptor (which may be referred to as an extracellular domain comprising an antigen binding region) comprises a tumor associated antigen or a pathogen-specific antigen binding domain. Antigens include carbohydrate antigens recognized by pattern-recognition receptors, such as Dectin-1. A tumor associated antigen may be of any kind so long as it is expressed on the cell surface of tumor cells. Exemplary embodiments of tumor associated antigens include CD19, CD20, carcinoembryonic antigen, alphafetoprotein, CA-125, MUC-1, CD56, EGFR, c-Met, AKT, Her2, Her3, epithelial tumor antigen, melanoma-associated antigen, mutated p53, mutated ras, and so forth. In certain embodiments, the CAR may be co-expressed with a cytokine to improve persistence when there is a low amount of tumor-associated antigen. For example, CAR may be co-expressed with IL-15.

The sequence of the open reading frame encoding the chimeric receptor can be obtained from a genomic DNA source, a cDNA source, or can be synthesized (e.g., via PCR), or combinations thereof. Depending upon the size of the genomic DNA and the number of introns, it may be desirable to use cDNA or a combination thereof as it is found that introns stabilize the mRNA. Also, it may be further advantageous to use endogenous or exogenous non-coding regions to stabilize the mRNA.

It is contemplated that the chimeric construct can be introduced into immune cells as naked DNA, a plasmid, or in a suitable vector. Methods of stably transfecting cells by electroporation using naked DNA or plasmids are known in the art. See, e.g., U.S. Pat. No. 6,410,319. Naked DNA generally refers to the DNA encoding a chimeric receptor contained in a plasmid expression vector in proper orientation for expression.

Alternatively, a viral vector (e.g., a retroviral vector, adenoviral vector, adeno-associated viral vector, or lentiviral vector) can be used to introduce the chimeric construct into immune cells. Suitable vectors for use in accordance with the method of the present disclosure are non-replicating in the immune cells. A large number of vectors are known that are based on viruses, where the copy number of the virus maintained in the cell is low enough to maintain the viability of the cell, such as, for example, vectors based on HIV, SV40, EBV, HSV, or BPV.

In some aspects, the antigen-specific binding, or recognition component is linked to one or more transmembrane and intracellular signaling domains. In some embodiments, the CAR includes a transmembrane domain fused to the extracellular domain of the CAR. In one embodiment, the transmembrane domain that naturally is associated with one of the domains in the CAR is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e. comprise at least the transmembrane region(s) of) the alpha, beta or zeta chain of the T-cell receptor, CD28, CD3 zeta, CD3 epsilon, CD3 gamma, CD3 delta, CD45, CD4, CD5, CD8, CD9, CD 16, CD22, CD33, CD37, CD64, CD80, CD86, CD 134, CD137, CD154, ICOS/CD278, GITR/CD357, NKG2D, and DAP molecules. Alternatively the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

In certain embodiments, the platform technologies disclosed herein to genetically modify immune cells, such as NK cells, comprise (i) non-viral gene transfer using an electroporation device (e.g., a nucleofector), (ii) CARs that signal through endodomains (e.g., CD28/CD3-ζ, CD137/CD3-ζ, or other combinations), (iii) CARs with variable lengths of extracellular domains connecting the antigen-recognition domain to the cell surface, and, in some cases, (iv) artificial antigen presenting cells (aAPC) derived from K562 to be able to robustly and numerically expand CAR⁺ immune cells (Singh et al., 2008; Singh et al., 2011).

B. T Cell Receptors

In some embodiments, the cells comprise genetically engineered antigen receptors, including recombinant TCRs and/or TCRs cloned from naturally occurring T cells. A “T cell receptor” or “TCR” refers to a molecule that contains a variable α and β chains (also known as TCRα and TCRβ, respectively) or a variable γ and δ chains (also known as TCRγ and TCRδ, respectively) and that is capable of specifically binding to an antigen peptide bound to a MHC receptor. In some embodiments, the TCR is in the αβ form. In alternative embodiments, the cells lack an engineered TCR; for example, endogenous TCR in the cells may target cancer or infectious diseases (e.g., CMV or EBV-specific T cells with endogenous TCR).

Typically, TCRs that exist in αβ and γδ forms are generally structurally similar, but T cells expressing them may have distinct anatomical locations or functions. A TCR can be found on the surface of a cell or in soluble form. Generally, a TCR is found on the surface of T cells (or T lymphocytes) where it is generally responsible for recognizing antigens bound to major histocompatibility complex (MHC) molecules. In some embodiments, a TCR also can contain a constant domain, a transmembrane domain and/or a short cytoplasmic tail (see, e.g., Janeway et al, 1997). For example, in some aspects, each chain of the TCR can possess one N-terminal immunoglobulin variable domain, one immunoglobulin constant domain, a transmembrane region, and a short cytoplasmic tail at the C-terminal end. In some embodiments, a TCR is associated with invariant proteins of the CD3 complex involved in mediating signal transduction. Unless otherwise stated, the term “TCR” should be understood to encompass functional TCR fragments thereof. The term also encompasses intact or full-length TCRs, including TCRs in the αβ form or γδ form.

Thus, for purposes herein, reference to a TCR includes any TCR or functional fragment, such as an antigen-binding portion of a TCR that binds to a specific antigenic peptide bound in an MHC molecule, i.e. MHC-peptide complex. An “antigen-binding portion” or antigen-binding fragment” of a TCR, which can be used interchangeably, refers to a molecule that contains a portion of the structural domains of a TCR, but that binds the antigen (e.g. MHC-peptide complex) to which the full TCR binds. In some cases, an antigen-binding portion contains the variable domains of a TCR, such as variable a chain and variable β chain of a TCR, sufficient to form a binding site for binding to a specific MHC-peptide complex, such as generally where each chain contains three complementarity determining regions.

In some embodiments, the variable domains of the TCR chains associate to form loops, or complementarity determining regions (CDRs) analogous to immunoglobulins, which confer antigen recognition and determine peptide specificity by forming the binding site of the TCR molecule and determine peptide specificity. Typically, like immunoglobulins, the CDRs are separated by framework regions (FRs) (see, e.g., Jores et al., 1990; Chothia et al., 1988; Lefranc et al., 2003). In some embodiments, CDR3 is the main CDR responsible for recognizing processed antigen, although CDR1 of the alpha chain has also been shown to interact with the N-terminal part of the antigenic peptide, whereas CDR1 of the beta chain interacts with the C-terminal part of the peptide. CDR2 is thought to recognize the MHC molecule. In some embodiments, the variable region of the β-chain can contain a further hypervariability (HV4) region.

In some embodiments, the TCR chains contain a constant domain. For example, like immunoglobulins, the extracellular portion of TCR chains (e.g., α-chain, β-chain) can contain two immunoglobulin domains, a variable domain (e.g., Vₐ or Vp; typically amino acids 1 to 116 based on Kabat numbering Kabat et al., “Sequences of Proteins of Immunological Interest, US Dept. Health and Human Services, Public Health Service National Institutes of Health, 1991, 5^{th} ed.) at the N-terminus, and one constant domain (e.g., a-chain constant domain or Ca, typically amino acids 117 to 259 based on Kabat, β-chain constant domain or Cp, typically amino acids 117 to 295 based on Kabat) adjacent to the cell membrane. For example, in some cases, the extracellular portion of the TCR formed by the two chains contains two membrane-proximal constant domains, and two membrane-distal variable domains containing CDRs. The constant domain of the TCR domain contains short connecting sequences in which a cysteine residue forms a disulfide bond, making a link between the two chains. In some embodiments, a TCR may have an additional cysteine residue in each of the α and β chains such that the TCR contains two disulfide bonds in the constant domains.

In some embodiments, the TCR chains can contain a transmembrane domain. In some embodiments, the transmembrane domain is positively charged. In some cases, the TCR chains contains a cytoplasmic tail. In some cases, the structure allows the TCR to associate with other molecules like CD3. For example, a TCR containing constant domains with a transmembrane region can anchor the protein in the cell membrane and associate with invariant subunits of the CD3 signaling apparatus or complex.

Generally, CD3 is a multi-protein complex that can possess three distinct chains (γ, δ, and ε) in mammals and the ζ-chain. For example, in mammals the complex can contain a CD3γ chain, a CD3δ chain, two CD3ε chains, and a homodimer of CD3ζ chains. The CD3γ, CD3δ, and CD3ε chains are highly related cell surface proteins of the immunoglobulin superfamily containing a single immunoglobulin domain. The transmembrane regions of the CD3γ, CD3δ, and CD3ε chains are negatively charged, which is a characteristic that allows these chains to associate with the positively charged T cell receptor chains. The intracellular tails of the CD3γ, CD3δ, and CD3ε chains each contain a single conserved motif known as an immunoreceptor tyrosine-based activation motif or ITAM, whereas each CD3ζ chain has three. Generally, ITAMs are involved in the signaling capacity of the TCR complex. These accessory molecules have negatively charged transmembrane regions and play a role in propagating the signal from the TCR into the cell. The CD3- and ζ-chains, together with the TCR, form what is known as the T cell receptor complex.

In some embodiments, the TCR may be a heterodimer of two chains a and β (or optionally γ and δ) or it may be a single chain TCR construct. In some embodiments, the TCR is a heterodimer containing two separate chains (α and β chains or γ and δ chains) that are linked, such as by a disulfide bond or disulfide bonds. In some embodiments, a TCR for a target antigen (e.g., a cancer antigen) is identified and introduced into the cells. In some embodiments, nucleic acid polymer encoding the TCR can be obtained from a variety of sources, such as by polymerase chain reaction (PCR) amplification of publicly available TCR DNA sequences. In some embodiments, the TCR is obtained from a biological source, such as from cells such as from a T cell (e.g. cytotoxic T cell), T cell hybridomas or other publicly available source. In some embodiments, the T cells can be obtained from in vivo isolated cells. In some embodiments, a high-affinity T cell clone can be isolated from a patient, and the TCR isolated. In some embodiments, the T cells can be a cultured T cell hybridoma or clone. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al., 2009 and Cohen et al., 2005). In some embodiments, phage display is used to isolate TCRs against a target antigen (see, e.g., Varela-Rohena et al., 2008 and Li, 2005). In some embodiments, the TCR or antigen-binding portion thereof can be synthetically generated from knowledge of the sequence of the TCR.

C. Antigen-Presenting Cells

Antigen-presenting cells may be cryopreserved with the medium encompassed herein. Antigen-presenting cells, which include macrophages, B lymphocytes, and dendritic cells, are distinguished by their expression of a particular MHC molecule. APCs internalize antigen and re-express a part of that antigen, together with the MHC molecule on their outer cell membrane. The MHC is a large genetic complex with multiple loci. The MHC loci encode two major classes of MHC membrane molecules, referred to as class I and class II MHCs. T helper lymphocytes generally recognize antigen associated with MHC class II molecules, and T cytotoxic lymphocytes recognize antigen associated with MHC class I molecules. In humans the MHC is referred to as the HLA complex and in mice the H-2 complex.

In some cases, aAPCs are useful in preparing therapeutic compositions and cell therapy products of the embodiments. For general guidance regarding the preparation and use of antigen-presenting systems, see, e.g., U.S. Pat. Nos. 6,225,042, 6,355,479, 6,362,001 and 6,790,662; U.S. Patent Application Publication Nos. 2009/0017000 and 2009/0004142; and International Publication No. WO2007/103009.

aAPC systems may comprise at least one exogenous assisting molecule. Any suitable number and combination of assisting molecules may be employed. The assisting molecule may be selected from assisting molecules such as co-stimulatory molecules and adhesion molecules. Exemplary co-stimulatory molecules include CD86, CD64 (FcγRI), 41BB ligand, and IL-21. Adhesion molecules may include carbohydrate-binding glycoproteins such as selectins, transmembrane binding glycoproteins such as integrins, calcium-dependent proteins such as cadherins, and single-pass transmembrane immunoglobulin (Ig) superfamily proteins, such as intercellular adhesion molecules (ICAMs), which promote, for example, cell-to-cell or cell-to-matrix contact. Exemplary adhesion molecules include LFA-3 and ICAMs, such as ICAM-1. Techniques, methods, and reagents useful for selection, cloning, preparation, and expression of exemplary assisting molecules, including co-stimulatory molecules and adhesion molecules, are exemplified in, e.g., U.S. Pat. Nos. 6,225,042, 6,355,479, and 6,362,001.

D. Antigens

Among the antigens targeted by the genetically engineered antigen receptors are those expressed in the context of a disease, condition, or cell type to be targeted via the adoptive cell therapy. Among the diseases and conditions are proliferative, neoplastic, and malignant diseases and disorders, including cancers and tumors, including hematologic cancers, cancers of the immune system, such as lymphomas, leukemias, and/or myelomas, such as B, T, and myeloid leukemias, lymphomas, and multiple myelomas. In some embodiments, the antigen is selectively expressed or overexpressed on cells of the disease or condition, e.g., the tumor or pathogenic cells, as compared to normal or non-targeted cells or tissues. In other embodiments, the antigen is expressed on normal cells and/or is expressed on the engineered cells.

Any suitable antigen may find use in the present method. Exemplary antigens include, but are not limited to, antigenic molecules from infectious agents, auto-/self-antigens, tumor-/cancer-associated antigens, and tumor neoantigens (Linnemann et al., 2015). In particular aspects, the antigens include BCMA, NY-ESO, EGFRvIII, Muc-1, Her2, CA-125, WT-1, Mage-A3, Mage-A4, Mage-A10, TRAIL/DR4, and CEA. In particular aspects, the antigens for the two or more antigen receptors include, but are not limited to, CD19, EBNA, WT1, CD123, NY-ESO, EGFRvIII, MUC1, HER2, CA-125, WT1, Mage-A3, Mage-A4, Mage-A10, TRAIL/DR4, and/or CEA. The sequences for these antigens are known in the art, for example, CD19 (Accession No. NG_007275.1), EBNA (Accession No. NG_002392.2), WT1 (Accession No. NG_009272.1), CD123 (Accession No. NC_000023.11), NY-ESO (Accession No. NC_000023.11), EGFRvIII (Accession No. NG_007726.3), MUC1 (Accession No. NG_029383.1), HER2 (Accession No. NG_007503.1), CA-125 (Accession No. NG_055257.1), WT1 (Accession No. NG_009272.1), Mage-A3 (Accession No. NG_013244.1), Mage-A4 (Accession No. NG_013245.1), Mage-A10 (Accession No. NC_000023.11), TRAIL/DR4 (Accession No. NC_000003.12), and/or CEA (Accession No. NC_000019.10).

Tumor-associated antigens may be derived from prostate, breast, colorectal, lung, pancreatic, renal, mesothelioma, ovarian, or melanoma cancers. Exemplary tumor-associated antigens or tumor cell-derived antigens include MAGE 1, 3, and MAGE 4 (or other MAGE antigens such as those disclosed in International Patent Publication No. WO99/40188); PRAME; BAGE; RAGE, Lage (also known as NY ESO 1); SAGE; and HAGE or GAGE. These non-limiting examples of tumor antigens are expressed in a wide range of tumor types such as melanoma, lung carcinoma, sarcoma, and bladder carcinoma. See, e.g., U.S. Pat. No. 6,544,518. Prostate cancer tumor-associated antigens include, for example, prostate specific membrane antigen (PSMA), prostate-specific antigen (PSA), prostatic acid phosphates, NKX3.1, and six-transmembrane epithelial antigen of the prostate (STEAP).

Other tumor associated antigens include Plu-1, HASH-1, HasH-2, Cripto and Criptin. Additionally, a tumor antigen may be a self peptide hormone, such as whole length gonadotrophin hormone releasing hormone (GnRH), a short 10 amino acid long peptide, useful in the treatment of many cancers.

Tumor antigens include tumor antigens derived from cancers that are characterized by tumor-associated antigen expression, such as HER-2/neu expression. Tumor-associated antigens of interest include lineage-specific tumor antigens such as the melanocyte-melanoma lineage antigens MART-1/Melan-A, gp100, gp75, mda-7, tyrosinase and tyrosinase-related protein. Illustrative tumor-associated antigens include, but are not limited to, tumor antigens derived from or comprising any one or more of, p53, Ras, c-Myc, cytoplasmic serine/threonine kinases (e.g., A-Raf, B-Raf, and C-Raf, cyclin-dependent kinases), MAGE-A1, MAGE-A2, MAGE-A3, MAGE-A4, MAGE-A6, MAGE-A10, MAGE-A12, MART-1, BAGE, DAM-6, -10, GAGE-1, -2, -8, GAGE-3, -4, -5, -6, -7B, NA88-A, MART-1, MC1R, Gp100, PSA, PSM, Tyrosinase, TRP-1, TRP-2, ART-4, CAMEL, CEA, Cyp-B, hTERT, hTRT, iCE, MUC1, MUC2, Phosphoinositide 3-kinases (PI3Ks), TRK receptors, PRAME, P15, RU1, RU2, SART-1, SART-3, Wilms' tumor antigen (WT1), AFP, -catenin/m, Caspase-8/m, CEA, CDK-4/m, ELF2M, GnT-V, G250, HSP70-2M, HST-2, KIAA0205, MUM-1, MUM-2, MUM-3, Myosin/m, RAGE, SART-2, TRP-2/INT2, 707-AP, Annexin II, CDC27/m, TPI/mbcr-abl, BCR-ABL, interferon regulatory factor 4 (IRF4), ETV6/AML, LDLR/FUT, Pml/RAR, Tumor-associated calcium signal transducer 1 (TACSTD1) TACSTD2, receptor tyrosine kinases (e.g., Epidermal Growth Factor receptor (EGFR) (in particular, EGFRvIII), platelet derived growth factor receptor (PDGFR), vascular endothelial growth factor receptor (VEGFR)), cytoplasmic tyrosine kinases (e.g., src-family, syk-ZAP70 family), integrin-linked kinase (ILK), signal transducers and activators of transcription STAT3, STATS, and STATE, hypoxia inducible factors (e.g., HIF-1 and HIF-2), Nuclear Factor-Kappa B (NF-B), Notch receptors (e.g., Notchl-4), c-Met, mammalian targets of rapamycin (mTOR), WNT, extracellular signal-regulated kinases (ERKs), and their regulatory subunits, PMSA, PR-3, MDM2, Mesothelin, renal cell carcinoma-5T4, SM22-alpha, carbonic anhydrases I (CAI) and IX (CAIX) (also known as G250), STEAD, TEL/AML1, GD2, proteinase3, hTERT, sarcoma translocation breakpoints, EphA2, ML-IAP, EpCAM, ERG (TMPRSS2 ETS fusion gene), NA17, PAX3, ALK, androgen receptor, cyclin B1, polysialic acid, MYCN, RhoC, GD3, fucosyl GM1, mesothelian, PSCA, sLe, PLAC1, GM3, BORIS, Tn, GLoboH, NY-BR-1, RGsS, SART3, STn, PAX5, OY-TES1, sperm protein 17, LCK, HMWMAA, AKAP-4, SSX2, XAGE 1, B7H3, legumain, TIE2, Page4, MAD-CT-1, FAP, MAD-CT-2, fos related antigen 1, CBX2, CLDN6, SPANX, TPTE, ACTL8, ANKRD30A, CDKN2A, MAD2L1, CTAGiB, SUNC1, LRRN1 and idiotype.

Antigens may include epitopic regions or epitopic peptides derived from genes mutated in tumor cells or from genes transcribed at different levels in tumor cells compared to normal cells, such as telomerase enzyme, survivin, mesothelin, mutated ras, bcr/abl rearrangement, Her2/neu, mutated or wild-type p53, cytochrome P450 1B1, and abnormally expressed intron sequences such as N-acetylglucosaminyltransferase-V; clonal rearrangements of immunoglobulin genes generating unique idiotypes in myeloma and B-cell lymphomas; tumor antigens that include epitopic regions or epitopic peptides derived from oncoviral processes, such as human papilloma virus proteins E6 and E7; Epstein bar virus protein LMP2; nonmutated oncofetal proteins with a tumor-selective expression, such as carcinoembryonic antigen and alphafetoprotein.

In other embodiments, an antigen is obtained or derived from a pathogenic microorganism or from an opportunistic pathogenic microorganism (also called herein an infectious disease microorganism), such as a virus, fungus, parasite, and bacterium. In certain embodiments, antigens derived from such a microorganism include full-length proteins.

Illustrative pathogenic organisms whose antigens are contemplated for use in the method described herein include human immunodeficiency virus (HIV), herpes simplex virus (HSV), respiratory syncytial virus (RSV), cytomegalovirus (CMV), Epstein-Barr virus (EBV), Influenza A, B, and C, vesicular stomatitis virus (VSV), vesicular stomatitis virus (VSV), polyomavirus (e.g., BK virus and JC virus), adenovirus, Staphylococcus species including Methicillin-resistant Staphylococcus aureus (MRSA), and Streptococcus species including Streptococcus pneumoniae. As would be understood by the skilled person, proteins derived from these and other pathogenic microorganisms for use as antigen as described herein and nucleotide sequences encoding the proteins may be identified in publications and in public databases such as GENBANK®, SWISS-PROT®, and TREMBL®.

Antigens derived from human immunodeficiency virus (HIV) include any of the HIV virion structural proteins (e.g., gp120, gp41, p17, p24), protease, reverse transcriptase, or HIV proteins encoded by tat, rev, nef, vif, vpr and vpu.

Antigens derived from herpes simplex virus (e.g., HSV 1 and HSV2) include, but are not limited to, proteins expressed from HSV late genes. The late group of genes predominantly encodes proteins that form the virion particle. Such proteins include the five proteins from (UL) which form the viral capsid: UL6, UL18, UL35, UL38 and the major capsid protein UL19, UL45, and UL27, each of which may be used as an antigen as described herein. Other illustrative HSV proteins contemplated for use as antigens herein include the ICP27 (H1, H2), glycoprotein B (gB) and glycoprotein D (gD) proteins. The HSV genome comprises at least 74 genes, each encoding a protein that could potentially be used as an antigen.

Antigens derived from cytomegalovirus (CMV) include CMV structural proteins, viral antigens expressed during the immediate early and early phases of virus replication, glycoproteins I and III, capsid protein, coat protein, lower matrix protein pp65 (ppUL83), p52 (ppUL44), IE1 and 1E2 (UL123 and UL122), protein products from the cluster of genes from UL128-UL150 (Rykman, et al., 2006), envelope glycoprotein B (gB), gH, gN, and pp150. As would be understood by the skilled person, CMV proteins for use as antigens described herein may be identified in public databases such as GENBANK®, SWISS-PROT®, and TREMBL® (see e.g., Bennekov et al., 2004; Loewendorf et al., 2010; Marschall et al., 2009).

Antigens derived from Epstein-Ban virus (EBV) that are contemplated for use in certain embodiments include EBV lytic proteins gp350 and gp110, EBV proteins produced during latent cycle infection including Epstein-Ban nuclear antigen (EBNA)-1, EBNA-2, EBNA-3A, EBNA-3B, EBNA-3C, EBNA-leader protein (EBNA-LP) and latent membrane proteins (LMP)-1, LMP-2A and LMP-2B (see, e.g., Lockey et al., 2008).

Antigens derived from respiratory syncytial virus (RSV) that are contemplated for use herein include any of the eleven proteins encoded by the RSV genome, or antigenic fragments thereof: NS 1, NS2, N (nucleocapsid protein), M (Matrix protein) SH, G and F (viral coat proteins), M2 (second matrix protein), M2-1 (elongation factor), M2-2 (transcription regulation), RNA polymerase, and phosphoprotein P.

Antigens derived from Vesicular stomatitis virus (VSV) that are contemplated for use include any one of the five major proteins encoded by the VSV genome, and antigenic fragments thereof: large protein (L), glycoprotein (G), nucleoprotein (N), phosphoprotein (P), and matrix protein (M) (see, e.g., Rieder et al., 1999).

Antigens derived from an influenza virus that are contemplated for use in certain embodiments include hemagglutinin (HA), neuraminidase (NA), nucleoprotein (NP), matrix proteins M1 and M2, NS1, NS2 (NEP), PA, PB1, PB1-F2, and PB2.

Exemplary viral antigens also include, but are not limited to, adenovirus polypeptides, alphavirus polypeptides, calicivirus polypeptides (e.g., a calicivirus capsid antigen), coronavirus polypeptides, distemper virus polypeptides, Ebola virus polypeptides, enterovirus polypeptides, flavivirus polypeptides, hepatitis virus (AE) polypeptides (a hepatitis B core or surface antigen, a hepatitis C virus E1 or E2 glycoproteins, core, or non-structural proteins), herpesvirus polypeptides (including a herpes simplex virus or varicella zoster virus glycoprotein), infectious peritonitis virus polypeptides, leukemia virus polypeptides, Marburg virus polypeptides, orthomyxovirus polypeptides, papilloma virus polypeptides, parainfluenza virus polypeptides (e.g., the hemagglutinin and neuraminidase polypeptides), paramyxovirus polypeptides, parvovirus polypeptides, pestivirus polypeptides, picorna virus polypeptides (e.g., a poliovirus capsid polypeptide), pox virus polypeptides (e.g., a vaccinia virus polypeptide), rabies virus polypeptides (e.g., a rabies virus glycoprotein G), reovirus polypeptides, retrovirus polypeptides, and rotavirus polypeptides.

In certain embodiments, the antigen may be bacterial antigens. In certain embodiments, a bacterial antigen of interest may be a secreted polypeptide. In other certain embodiments, bacterial antigens include antigens that have a portion or portions of the polypeptide exposed on the outer cell surface of the bacteria.

Antigens derived from Staphylococcus species including Methicillin-resistant Staphylococcus aureus (MRSA) that are contemplated for use include virulence regulators, such as the Agr system, Sar and Sae, the Arl system, Sar homologues (Rot, MgrA, SarS, SarR, SarT, SarU, SarV, SarX, SarZ and TcaR), the Srr system and TRAP. Other Staphylococcus proteins that may serve as antigens include Clp proteins, HtrA, MsrR, aconitase, CcpA, SvrA, Msa, CfvA and CfvB (see, e.g., Staphylococcus: Molecular Genetics, 2008 Caister Academic Press, Ed. Jodi Lindsay). The genomes for two species of Staphylococcus aureus (N315 and Mu50) have been sequenced and are publicly available, for example at PATRIC (PATRIC: The VBI PathoSystems Resource Integration Center, Snyder et al., 2007). As would be understood by the skilled person, Staphylococcus proteins for use as antigens may also be identified in other public databases such as GenBank®, Swiss-Prot®, and TrEMBL®.

Antigens derived from Streptococcus pneumoniae that are contemplated for use in certain embodiments described herein include pneumolysin, PspA, choline-binding protein A (CbpA), NanA, NanB, SpnHL, PavA, LytA, Pht, and pilin proteins (RrgA; RrgB; RrgC). Antigenic proteins of Streptococcus pneumoniae are also known in the art and may be used as an antigen in some embodiments (see, e.g., Zysk et al., 2000). The complete genome sequence of a virulent strain of Streptococcus pneumoniae has been sequenced and, as would be understood by the skilled person, S. pneumoniae proteins for use herein may also be identified in other public databases such as GENBANK®, SWISS-PROT®, and TREMBL®. Proteins of particular interest for antigens according to the present disclosure include virulence factors and proteins predicted to be exposed at the surface of the pneumococci (see, e.g., Frolet et al., 2010).

Examples of bacterial antigens that may be used as antigens include, but are not limited to, Actinomyces polypeptides, Bacillus polypeptides, Bacteroides polypeptides, Bordetella polypeptides, Bartonella polypeptides, Borrelia polypeptides (e.g., B. burgdorferi OspA), Brucella polypeptides, Campylobacter polypeptides, Capnocytophaga polypeptides, Chlamydia polypeptides, Corynebacterium polypeptides, Coxiella polypeptides, Dermatophilus polypeptides, Enterococcus polypeptides, Ehrlichia polypeptides, Escherichia polypeptides, Francisella polypeptides, Fusobacterium polypeptides, Haemobartonella polypeptides, Haemophilus polypeptides (e.g., H. influenzae type b outer membrane protein), Helicobacter polypeptides, Klebsiella polypeptides, L-form bacteria polypeptides, Leptospira polypeptides, Listeria polypeptides, Mycobacteria polypeptides, Mycoplasma polypeptides, Neisseria polypeptides, Neorickettsia polypeptides, Nocardia polypeptides, Pasteurella polypeptides, Peptococcus polypeptides, Peptostreptococcus polypeptides, Pneumococcus polypeptides (i.e., S. pneumoniae polypeptides) (see description herein), Proteus polypeptides, Pseudomonas polypeptides, Rickettsia polypeptides, Rochalimaea polypeptides, Salmonella polypeptides, Shigella polypeptides, Staphylococcus polypeptides, group A streptococcus polypeptides (e.g., S. pyogenes M proteins), group B streptococcus (S. agalactiae) polypeptides, Treponema polypeptides, and Yersinia polypeptides (e.g., Ypestis F1 and V antigens).

Examples of fungal antigens include, but are not limited to, Absidia polypeptides, Acremonium polypeptides, Alternaria polypeptides, Aspergillus polypeptides, Basidiobolus polypeptides, Bipolaris polypeptides, Blastomyces polypeptides, Candida polypeptides, Coccidioides polypeptides, Conidiobolus polypeptides, Cryptococcus polypeptides, Curvalaria polypeptides, Epidermophyton polypeptides, Exophiala polypeptides, Geotrichum polypeptides, Histoplasma polypeptides, Madurella polypeptides, Malassezia polypeptides, Microsporum polypeptides, Moniliella polypeptides, Mortierella polypeptides, Mucor polypeptides, Paecilomyces polypeptides, Penicillium polypeptides, Phialemonium polypeptides, Phialophora polypeptides, Prototheca polypeptides, Pseudallescheria polypeptides, Pseudomicrodochium polypeptides, Pythium polypeptides, Rhinosporidium polypeptides, Rhizopus polypeptides, Scolecobasidium polypeptides, Sporothrix polypeptides, Stemphylium polypeptides, Trichophyton polypeptides, Trichosporon polypeptides, and Xylohypha polypeptides.

Examples of protozoan parasite antigens include, but are not limited to, Babesia polypeptides, Balantidium polypeptides, Besnoitia polypeptides, Cryptosporidium polypeptides, Eimeria polypeptides, Encephalitozoon polypeptides, Entamoeba polypeptides, Giardia polypeptides, Hammondia polypeptides, Hepatozoon polypeptides, Isospora polypeptides, Leishmania polypeptides, Microsporidia polypeptides, Neospora polypeptides, Nosema polypeptides, Pentatrichomonas polypeptides, Plasmodium polypeptides. Examples of helminth parasite antigens include, but are not limited to, Acanthocheilonema polypeptides, Aelurostrongylus polypeptides, Ancylostoma polypeptides, Angiostrongylus polypeptides, Ascaris polypeptides, Brugia polypeptides, Bunostomum polypeptides, Capillaria polypeptides, Chabertia polypeptides, Cooperia polypeptides, Crenosoma polypeptides, Dictyocaulus polypeptides, Dioctophyme polypeptides, Dipetalonema polypeptides, Diphyllobothrium polypeptides, Diplydium polypeptides, Dirofilaria polypeptides, Dracunculus polypeptides, Enterobius polypeptides, Filaroides polypeptides, Haemonchus polypeptides, Lagochilascaris polypeptides, Loa polypeptides, Mansonella polypeptides, Muellerius polypeptides, Nanophyetus polypeptides, Necator polypeptides, Nematodirus polypeptides, Oesophagostomum polypeptides, Onchocerca polypeptides, Opisthorchis polypeptides, Ostertagia polypeptides, Parafilaria polypeptides, Paragonimus polypeptides, Parascaris polypeptides, Physaloptera polypeptides, Protostrongylus polypeptides, Setaria polypeptides, Spirocerca polypeptides Spirometra polypeptides, Stephanofilaria polypeptides, Strongyloides polypeptides, Strongylus polypeptides, Thelazia polypeptides, Toxascaris polypeptides, Toxocara polypeptides, Trichinella polypeptides, Trichostrongylus polypeptides, Trichuris polypeptides, Uncinaria polypeptides, and Wuchereria polypeptides. (e.g., P. falciparum circumsporozoite (PfCSP)), sporozoite surface protein 2 (PfSSP2), carboxyl terminus of liver state antigen 1 (PfLSA1 c-term), and exported protein 1 (PfExp-1), Pneumocystis polypeptides, Sarcocystis polypeptides, Schistosoma polypeptides, Theileria polypeptides, Toxoplasma polypeptides, and Trypanosoma polypeptides.

Examples of ectoparasite antigens include, but are not limited to, polypeptides (including antigens as well as allergens) from fleas; ticks, including hard ticks and soft ticks; flies, such as midges, mosquitoes, sand flies, black flies, horse flies, horn flies, deer flies, tsetse flies, stable flies, myiasis-causing flies and biting gnats; ants; spiders, lice; mites; and true bugs, such as bed bugs and kissing bugs.

E. Suicide Genes

In some embodiments, the cells encompass nucleic acids that express one or more suicide genes. The cells may be manipulated to express a suicide gene either before or after cryopreservation and thawing. The CAR of the exemplary immune cells of the present disclosure may comprise one or more suicide genes. The term “suicide gene” as used herein is defined as a gene which, upon administration of a prodrug, effects transition of a gene product to a compound which kills its host cell. Examples of suicide gene/prodrug combinations which may be used are Herpes Simplex Virus-thymidine kinase (HSV-tk) and ganciclovir, acyclovir, or FIAU; oxidoreductase and cycloheximide; cytosine deaminase and 5-fluorocytosine; thymidine kinase thymidilate kinase (Tdk::Tmk) and AZT; and deoxycytidine kinase and cytosine arabinoside.

The E. coli purine nucleoside phosphorylase, a so-called suicide gene which converts the prodrug 6-methylpurine deoxyriboside to toxic purine 6-methylpurine. Other examples of suicide genes used with prodrug therapy are the E. coli cytosine deaminase gene and the HSV thymidine kinase gene.

Exemplary suicide genes include CD20, mutant TNF-alpha (for example, a non-secretable mutant), CD52, EGFRv3, or inducible caspase 9. In one embodiment, a truncated version of EGFR variant III (EGFRv3) may be used as a suicide antigen which can be ablated by Cetuximab. Further suicide genes known in the art that may be used in the present disclosure include Purine nucleoside phosphorylase (PNP), Cytochrome p450 enzymes (CYP), Carboxypeptidases (CP), Carboxylesterase (CE), Nitroreductase (NTR), Guanine Ribosyltransferase (XGRTP), Glycosidase enzymes, Methionine-α,γ-lyase (MET), and Thymidine phosphorylase (TP).

F. Methods of Delivery

The cells encompassed herein may harbor a recombinant vector, either before or following thawing after cryopreservation. One of skill in the art would be well-equipped to construct a vector through standard recombinant techniques (see, for example, Sambrook et al., 2001 and Ausubel et al., 1996, both incorporated herein by reference) for the expression of the antigen receptors of the present disclosure. Vectors include but are not limited to, plasmids, cosmids, viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors, parvovirus vectors, polio virus vectors, vesicular stomatitis virus vectors, maraba virus vectors and group B adenovirus enadenotucirev vectors.

a. Viral Vectors

Viral vectors encoding an antigen receptor may be provided in certain aspects of the present disclosure. In generating recombinant viral vectors, non-essential genes are typically replaced with a gene or coding sequence for a heterologous (or non-native) protein. A viral vector is a kind of expression construct that utilizes viral sequences to introduce nucleic acid and possibly proteins into a cell. The ability of certain viruses to infect cells or enter cells via receptor mediated-endocytosis, and to integrate into host cell genomes and express viral genes stably and efficiently have made them attractive candidates for the transfer of foreign nucleic acids into cells (e.g., mammalian cells). Non-limiting examples of virus vectors that may be used to deliver a nucleic acid of certain aspects of the present invention are described below.

Lentiviruses are complex retroviruses, which, in addition to the common retroviral genes gag, pol, and env, contain other genes with regulatory or structural function. Lentiviral vectors are well known in the art (see, for example, U.S. Pat. Nos. 6,013,516 and 5,994,136).

Recombinant lentiviral vectors are capable of infecting non-dividing cells and can be used for both in vivo and ex vivo gene transfer and expression of nucleic acid sequences. For example, recombinant lentivirus capable of infecting a non-dividing cell—wherein a suitable host cell is transfected with two or more vectors carrying the packaging functions, namely gag, pol and env, as well as rev and tat—is described in U.S. Pat. No. 5,994,136, incorporated herein by reference.

b. Regulatory Elements

Expression cassettes included in vectors useful in the present disclosure in particular contain (in a 5′-to-3′ direction) a eukaryotic transcriptional promoter operably linked to a protein-coding sequence, splice signals including intervening sequences, and a transcriptional termination/polyadenylation sequence. The promoters and enhancers that control the transcription of protein encoding genes in eukaryotic cells are composed of multiple genetic elements. The cellular machinery is able to gather and integrate the regulatory information conveyed by each element, allowing different genes to evolve distinct, often complex patterns of transcriptional regulation. A promoter used in the context of the present disclosure includes constitutive, inducible, and tissue-specific promoters.

c. Promoter/Enhancers

The expression constructs provided herein comprise a promoter to drive expression of the antigen receptor. A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30110 bp-upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the βlactamase (penicillinase), lactose and tryptophan (trp-) promoter systems. In addition to producing nucleic acid sequences of promoters and enhancers synthetically, sequences may be produced using recombinant cloning and/or nucleic acid amplification technology, including PCR™, in connection with the compositions disclosed herein. Furthermore, it is contemplated that the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally, any promoter/enhancer combination (as per, for example, the Eukaryotic Promoter Data Base EPDB, through world wide web at epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Non-limiting examples of promoters include early or late viral promoters, such as, SV40 early or late promoters, cytomegalovirus (CMV) immediate early promoters, Rous Sarcoma Virus (RSV) early promoters; eukaryotic cell promoters, such as, e. g., beta actin promoter, GADPH promoter, metallothionein promoter; and concatenated response element promoters, such as cyclic AMP response element promoters (cre), serum response element promoter (sre), phorbol ester promoter (TPA) and response element promoters (tre) near a minimal TATA box. It is also possible to use human growth hormone promoter sequences (e.g., the human growth hormone minimal promoter described at Genbank, accession no. X05244, nucleotide 283-341) or a mouse mammary tumor promoter (available from the ATCC, Cat. No. ATCC 45007). In certain embodiments, the promoter is CMV IE, dectin-1, dectin-2, human CD11c, F4/80, SM22, RSV, SV40, Ad MLP, beta-actin, MHC class I or MHC class II promoter, however any other promoter that is useful to drive expression of the therapeutic gene is applicable to the practice of the present disclosure.

In certain aspects, methods of the disclosure also concern enhancer sequences, i.e., nucleic acid sequences that increase a promoter's activity and that have the potential to act in cis, and regardless of their orientation, even over relatively long distances (up to several kilobases away from the target promoter). However, enhancer function is not necessarily restricted to such long distances as they may also function in close proximity to a given promoter.

d. Initiation Signals and Linked Expression

A specific initiation signal also may be used in the expression constructs provided in the present disclosure for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments, the use of internal ribosome entry sites (IRES) elements are used to create multigene, or polycistronic, messages. IRES elements are able to bypass the ribosome scanning model of 5′ methylated Cap dependent translation and begin translation at internal sites. IRES elements from two members of the picornavirus family (polio and encephalomyocarditis) have been described, as well an IRES from a mammalian message. IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Additionally, certain 2A sequence elements could be used to create linked-or co-expression of genes in the constructs provided in the present disclosure. For example, cleavage sequences could be used to co-express genes by linking open reading frames to form a single cistron. An exemplary cleavage sequence is the F2A (Foot-and-mouth disease virus 2A) or a “2A-like” sequence (e.g., Thosea asigna virus 2A; T2A).

e. Origins of Replication

In order to propagate a vector in a host cell, it may contain one or more origins of replication sites (often termed “ori”), for example, a nucleic acid sequence corresponding to oriP of EBV as described above or a genetically engineered oriP with a similar or elevated function in programming, which is a specific nucleic acid sequence at which replication is initiated. Alternatively a replication origin of other extra-chromosomally replicating virus as described above or an autonomously replicating sequence (ARS) can be employed.

f. Selection and Screenable Markers

In some embodiments, cells containing a construct of the present disclosure may be identified in vitro or in vivo by including a marker in the expression vector. Such markers would confer an identifiable change to the cell permitting easy identification of cells containing the expression vector. Generally, a selection marker is one that confers a property that allows for selection. A positive selection marker is one in which the presence of the marker allows for its selection, while a negative selection marker is one in which its presence prevents its selection. An example of a positive selection marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selection markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes as negative selection markers such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selection and screenable markers are well known to one of skill in the art.

g. Other Methods of Nucleic Acid Delivery

In addition to viral delivery of the nucleic acids encoding the antigen receptor, the following are additional methods of recombinant gene delivery to a given host cell and are thus considered in the present disclosure.

Introduction of a nucleic acid, such as DNA or RNA, into the immune cells of the current disclosure may use any suitable methods for nucleic acid delivery for transformation of a cell, as described herein or as would be known to one of ordinary skill in the art. Such methods include, but are not limited to, direct delivery of DNA such as by ex vivo transfection, by injection, including microinjection); by electroporation; by calcium phosphate precipitation; by using DEAE-dextran followed by polyethylene glycol; by direct sonic loading; by liposome mediated transfection and receptor-mediated transfection; by microprojectile bombardment; by agitation with silicon carbide fibers; by Agrobacterium-mediated transformation; by desiccation/inhibition-mediated DNA uptake, and any combination of such methods. Through the application of techniques such as these, organelle(s), cell(s), tissue(s) or organism(s) may be stably or transiently transformed.

G. Modification of Gene Expression

In some embodiments, the immune cells of the present disclosure that are cryopreserved are modified to have altered expression of certain genes such as glucocorticoid receptor, TGFβ receptor (e.g., TGFβ-RII), and/or CISH. In one embodiment, the immune cells may be modified to express a dominant negative TGFβ receptor II (TGFβRIIDN) which can function as a cytokine sink to deplete endogenous TGFβ.

Cytokine signaling is essential for the normal function of hematopoietic cells. The SOCS family of proteins plays an important role in the negative regulation of cytokine signaling, acting as an intrinsic brake. CIS, a member of the SOCS family of proteins encoded by the CISH gene, has been identified as an important checkpoint molecule in NK cells in mice. Thus, in some embodiments, the present disclosure concerns the knockout of CISH in immune cells to improve cytotoxicity of NK cells and CD8⁺ T cells, for example. This approach may be used alone or in combination with other checkpoint inhibitors to improve anti-tumor activity.

In some embodiments, the altered gene expression is carried out by effecting a disruption in the gene, such as a knock-out, insertion, missense or frameshift mutation, such as biallelic frameshift mutation, deletion of all or part of the gene, e.g., one or more exon or portion therefore, and/or knock-in. For example, the altered gene expression can be effected by sequence-specific or targeted nucleases, including DNA-binding targeted nucleases such as zinc finger nucleases (ZFN) and transcription activator-like effector nucleases (TALENs), and RNA-guided nucleases such as a CRISPR-associated nuclease (Cas), specifically designed to be targeted to the sequence of the gene or a portion thereof.

In some embodiments, the alteration of the expression, activity, and/or function of the gene is carried out by disrupting the gene. In some aspects, the gene is modified so that its expression is reduced by at least at or about 20, 30, or 40%, generally at least at or about 50, 60, 70, 80, 90, or 95% as compared to the expression in the absence of the gene modification or in the absence of the components introduced to effect the modification.

In some embodiments, the alteration is transient or reversible, such that expression of the gene is restored at a later time. In other embodiments, the alteration is not reversible or transient, e.g., is permanent.

In some embodiments, gene alteration is carried out by induction of one or more double-stranded breaks and/or one or more single-stranded breaks in the gene, typically in a targeted manner. In some embodiments, the double-stranded or single-stranded breaks are made by a nuclease, e.g. an endonuclease, such as a gene-targeted nuclease. In some aspects, the breaks are induced in the coding region of the gene, e.g. in an exon. For example, in some embodiments, the induction occurs near the N-terminal portion of the coding region, e.g. in the first exon, in the second exon, or in a subsequent exon.

In some aspects, the double-stranded or single-stranded breaks undergo repair via a cellular repair process, such as by non-homologous end-joining (NHEJ) or homology-directed repair (HDR). In some aspects, the repair process is error-prone and results in disruption of the gene, such as a frameshift mutation, e.g., biallelic frameshift mutation, which can result in complete knockout of the gene. For example, in some aspects, the disruption comprises inducing a deletion, mutation, and/or insertion. In some embodiments, the disruption results in the presence of an early stop codon. In some aspects, the presence of an insertion, deletion, translocation, frameshift mutation, and/or a premature stop codon results in disruption of the expression, activity, and/or function of the gene.

In some embodiments, gene alteration is achieved using antisense techniques, such as by RNA interference (RNAi), short interfering RNA (siRNA), short hairpin (shRNA), and/or ribozymes are used to selectively suppress or repress expression of the gene. siRNA technology is RNAi which employs a double-stranded RNA molecule having a sequence homologous with the nucleotide sequence of mRNA which is transcribed from the gene, and a sequence complementary with the nucleotide sequence. siRNA generally is homologous/complementary with one region of mRNA which is transcribed from the gene, or may be siRNA including a plurality of RNA molecules which are homologous/complementary with different regions. In some aspects, the siRNA is comprised in a polycistronic construct.

1. ZFPs and ZFNs

In some embodiments, the DNA-targeting molecule includes a DNA-binding protein such as one or more zinc finger protein (ZFP) or transcription activator-like protein (TAL), fused to an effector protein such as an endonuclease. Examples include ZFNs, TALEs, and TALENs.

In some embodiments, the DNA-targeting molecule comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion. The term zinc finger DNA binding protein is often abbreviated as zinc finger protein or ZFP. Among the ZFPs are artificial ZFP domains targeting specific DNA sequences, typically 9-18 nucleotides long, generated by assembly of individual fingers.

ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice.

In some embodiments, the DNA-targeting molecule is or comprises a zinc-finger DNA binding domain fused to a DNA cleavage domain to form a zinc-finger nuclease (ZFN). In some embodiments, fusion proteins comprise the cleavage domain (or cleavage half-domain) from at least one Type liS restriction enzyme and one or more zinc finger binding domains, which may or may not be engineered. In some embodiments, the cleavage domain is from the Type liS restriction endonuclease Fok I. Fok I generally catalyzes double-stranded cleavage of DNA, at 9 nucleotides from its recognition site on one strand and 13 nucleotides from its recognition site on the other.

Many gene-specific engineered zinc fingers are available commercially. For example, Sangamo Biosciences (Richmond, Calif., USA) has developed a platform (CompoZr) for zinc-finger construction in partnership with Sigma-Aldrich (St. Louis, Mo., USA), allowing investigators to bypass zinc-finger construction and validation altogether, and provides specifically targeted zinc fingers for thousands of proteins (Gaj et al., Trends in Biotechnology, 2013, 31(7), 397-405). In some embodiments, commercially available zinc fingers are used or are custom designed. (See, for example, Sigma-Aldrich catalog numbers CSTZFND, CSTZFN, CTil-1KT, and PZD0020).

2. TALs, TALEs and TALENs

In some embodiments, the DNA-targeting molecule comprises a naturally occurring or engineered (non-naturally occurring) transcription activator-like protein (TAL) DNA binding domain, such as in a transcription activator-like protein effector (TALE) protein, See, e.g., U.S. Patent Publication No. 2011/0301073, incorporated by reference in its entirety herein.

A TALE DNA binding domain or TALE is a polypeptide comprising one or more TALE repeat domains/units. The repeat domains are involved in binding of the TALE to its cognate target DNA sequence. A single “repeat unit” (also referred to as a “repeat”) is typically 33-35 amino acids in length and exhibits at least some sequence homology with other TALE repeat sequences within a naturally occurring TALE protein. Each TALE repeat unit includes 1 or 2 DNA-binding residues making up the Repeat Variable Diresidue (RVD), typically at positions 12 and/or 13 of the repeat. The natural (canonical) code for DNA recognition of these TALEs has been determined such that an HD sequence at positions 12 and 13 leads to a binding to cytosine (C), NG binds to T, NI to A, NN binds to G or A, and NO binds to T and non-canonical (atypical) RVDs are also known. In some embodiments, TALEs may be targeted to any gene by design of TAL arrays with specificity to the target DNA sequence. The target sequence generally begins with a thymidine.

In some embodiments, the molecule is a DNA binding endonuclease, such as a TALE nuclease (TALEN). In some aspects the TALEN is a fusion protein comprising a DNA-binding domain derived from a TALE and a nuclease catalytic domain to cleave a nucleic acid target sequence.

In some embodiments, the TALEN recognizes and cleaves the target sequence in the gene. In some aspects, cleavage of the DNA results in double-stranded breaks. In some aspects the breaks stimulate the rate of homologous recombination or non-homologous end joining (NHEJ). Generally, NHEJ is an imperfect repair process that often results in changes to the DNA sequence at the site of the cleavage. In some aspects, repair mechanisms involve rejoining of what remains of the two DNA ends through direct re-ligation or via the so-called microhomology-mediated end joining. In some embodiments, repair via NHEJ results in small insertions or deletions and can be used to disrupt and thereby repress the gene. In some embodiments, the modification may be a substitution, deletion, or addition of at least one nucleotide. In some aspects, cells in which a cleavage-induced mutagenesis event, i.e. a mutagenesis event consecutive to an NHEJ event, has occurred can be identified and/or selected by well-known methods in the art.

In some embodiments, TALE repeats are assembled to specifically target a gene. (Gaj et al., 2013). A library of TALENs targeting 18,740 human protein-coding genes has been constructed (Kim et al., 2013). Custom-designed TALE arrays are commercially available through Cellectis Bioresearch (Paris, France), Transposagen Biopharmaceuticals (Lexington, Ky., USA), and Life Technologies (Grand Island, N.Y., USA). Specifically, TALENs that target CD38 are commercially available (See Gencopoeia, catalog numbers HTN222870-1, HTN222870-2, and HTN222870-3). Exemplary molecules are described, e.g., in U.S. Patent Publication Nos. US 2014/0120622, and 2013/0315884.

In some embodiments the TALEN s are introduced as trans genes encoded by one or more plasmid vectors. In some aspects, the plasmid vector can contain a selection marker which provides for identification and/or selection of cells which received said vector.

3. RGENs (CRISPR/Cas Systems)

In some embodiments, the alteration is carried out using one or more DNA-binding nucleic acids, such as alteration via an RNA-guided endonuclease (RGEN). For example, the alteration can be carried out using clustered regularly interspaced short palindromic repeats (CRISPR) and CRISPR-associated (Cas) proteins. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions or alterations as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, sheep, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Exemplary gRNA sequences for NR3CS (glucocorticoid receptor) include Ex3 NR3C1 sG1 5-TGC TGT TGA GGA GCT GGA-3 (SEQ ID NO:1) and Ex3 NR3C1 sG2 5-AGC ACA CCA GGC AGA GTT-3 (SEQ ID NO:2). Exemplary gRNA sequences for TGF-beta receptor 2 include EX3 TGFBR2 sG1 5-CGG CTG AGG AGC GGA AGA-3 (SEQ ID NO:3) and EX3 TGFBR2 sG2 5-TGG-AGG-TGA-GCA-ATC-CCC-3 (SEQ ID NO:4). The T7 promoter, target sequence, and overlap sequence may have the sequence TTAATACGACTCACTATAGG (SEQ ID NO:5)+target sequence+gttttagagctagaaatagc (SEQ ID NO:6).

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

### IV. Methods of Treatment

In some embodiments, the present disclosure provides methods for immunotherapy comprising administering an effective amount of the cryopreserved cells of the present disclosure following thawing. In one embodiments, a medical disease or disorder is treated by transfer of a cell population previously cryopreserved, such as an NK cell population that elicits an immune response. The cells following thawing may or may not be washed to remove substantially all of the cryopreservation medium prior to administration of the cells to an individual. The cells following thawing may be diluted without washing and infused. The cells may be delivered to an individual substantially immediately upon thawing, or there may be a delay before delivery on the order of 1-24 hours or 1 or more days, for example, including if the cells were washed before infusion. The delivery may be by any route and may depend on the medical condition being treated. The delivery may be local or systemic. With respect to infusion volumes of doses of cells being delivered, the infusion volume may or may not depend on whether or not the subject has already received a dose of cells. For example, a first dose of cells may or may not be greater in volume than a subsequent dose. Multiple infusion volumes may be of the same volume. In some embodiments, the infusion volume of the cells is 1, 5, 10, 15, 20, 30, 40, 50, 60, 70, 75, 80, 90, 100, 125, 150, 175, 200, 225, 250, 275, or 300 or more mL. The liquid in which the cells are suspended for infusion may be of any kind. In specific embodiments, the liquid is PLASMA-LYTE A or a similar solution. The liquid in which the cells are suspended for infusion may or may not comprise human serum albumin, for example. Albumin is a cryoprotectant that can also be used as a non-serum alternative, so it has dual effects. Prior to delivery to an individual in need thereof, the thawed cells may be tested for one or more characteristic, such as the presence of microbes, for example by contamination; viability; cell count, and so forth. In specific embodiments, the cells for infusion are comprised in a solution that comprises one or more other therapeutic agents than the cells themselves.

In certain embodiments of the present disclosure, cancer or infection is treated by transfer of a cryopreserved and thawed population, such as an NK cell population that elicits an immune response. Provided herein are methods for treating or delaying progression of cancer in an individual comprising administering to the individual an effective amount an antigen-specific cell therapy. The present methods may be applied for the treatment of immune disorders, solid cancers, hematologic cancers, viral infections, and regenerative medicine.

Tumors for which the present treatment methods are useful include any malignant cell type, such as those found in a solid tumor or a hematological tumor. Exemplary solid tumors can include, but are not limited to, a tumor of an organ selected from the group consisting of pancreas, colon, cecum, stomach, brain, head, neck, ovary, kidney, larynx, sarcoma, lung, bladder, melanoma, prostate, and breast. Exemplary hematological tumors include tumors of the bone marrow, T or B cell malignancies, leukemias, lymphomas, blastomas, myelomas, and the like. Further examples of cancers that may be treated using the methods provided herein include, but are not limited to, lung cancer (including small-cell lung cancer, non-small cell lung cancer, adenocarcinoma of the lung, and squamous carcinoma of the lung), cancer of the peritoneum, gastric or stomach cancer (including gastrointestinal cancer and gastrointestinal stromal cancer), pancreatic cancer, cervical cancer, ovarian cancer, liver cancer, bladder cancer, breast cancer, colon cancer, colorectal cancer, endometrial or uterine carcinoma, salivary gland carcinoma, kidney or renal cancer, prostate cancer, vulval cancer, thyroid cancer, various types of head and neck cancer, and melanoma.

The cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; lentigo malignant melanoma; acral lentiginous melanomas; nodular melanomas; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; hodgkin's disease; hodgkin's; paragranuloma; malignant lymphoma, small lymphocytic; malignant lymphoma, large cell, diffuse; malignant lymphoma, follicular; mycosis fungoides; other specified non-hodgkin's lymphomas; B-cell lymphoma; low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; Waldenstrom's macroglobulinemia; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; hairy cell leukemia; chronic lymphocytic leukemia (CLL); acute lymphoblastic leukemia (ALL); acute myeloid leukemia (AML); and chronic myeloblastic leukemia.

Particular embodiments concern methods of treatment of leukemia. Leukemia is a cancer of the blood or bone marrow and is characterized by an abnormal proliferation (production by multiplication) of blood cells, usually white blood cells (leukocytes) but can involve red blood cells (erythroleukemia). It is part of the broad group of diseases called hematological neoplasms. Leukemia is a broad term covering a spectrum of diseases. Leukemia is clinically and pathologically split into its acute and chronic forms.

In certain embodiments of the present disclosure, immune cells are delivered to an individual in need thereof, such as an individual that has cancer or an infection. The cells then enhance the individual's immune system to attack the respective cancer or pathologic cells. In some cases, the individual is provided with one or more doses of the immune cells. In cases where the individual is provided with two or more doses of the immune cells, the duration between the administrations should be sufficient to allow time for propagation in the individual, and in specific embodiments the duration between doses is 1, 2, 3, 4, 5, 6, 7, or more days.

Certain embodiments of the present disclosure provide methods for treating or preventing an immune-mediated disorder. In one embodiment, the subject has an autoimmune disease. Non-limiting examples of autoimmune diseases include: alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac spate-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, nephrotic syndrome (such as minimal change disease, focal glomerulosclerosis, or mebranous nephropathy), pemphigus vulgaris, pernicious anemia, polyarteritis nodosa, polychondritis, polyglandular syndromes, polymyalgia rheumatica, polymyositis and dermatomyositis, primary agammaglobulinemia, primary biliary cirrhosis, psoriasis, psoriatic arthritis, Raynaud's phenomenon, Reiter's syndrome, Rheumatoid arthritis, sarcoidosis, scleroderma, Sjogren's syndrome, stiff-man syndrome, systemic lupus erythematosus, lupus erythematosus, ulcerative colitis, uveitis, vasculitides (such as polyarteritis nodosa, takayasu arteritis, temporal arteritis/giant cell arteritis, or dermatitis herpetiformis vasculitis), vitiligo, and Wegener's granulomatosis. Thus, some examples of an autoimmune disease that can be treated using the methods disclosed herein include, but are not limited to, multiple sclerosis, rheumatoid arthritis, systemic lupus erythematosis, type I diabetes mellitus, Crohn's disease; ulcerative colitis, myasthenia gravis, glomerulonephritis, ankylosing spondylitis, vasculitis, or psoriasis. The subject can also have an allergic disorder such as Asthma.

In yet another embodiment, the subject is the recipient of a transplanted organ or stem cells and immune cells are used to prevent and/or treat rejection. In particular embodiments, the subject has or is at risk of developing graft versus host disease. GVHD is a possible complication of any transplant that uses or contains stem cells from either a related or an unrelated donor. There are two kinds of GVHD, acute and chronic. Acute GVHD appears within the first three months following transplantation. Signs of acute GVHD include a reddish skin rash involving small areas of the body initially (chest, back, arms, legs) and that may spread and become more severe encompassing >80% of the body, with peeling or blistering skin. Acute GVHD can also affect the gastrointestinal (GI) tract, in which casenausea and vomiting (upper GI GVHD) and/or abdominal cramping and diarrhea (lower GI GVHD) are present. Yellowing of the skin and eyes (jaundice) indicates that acute GVHD has affected the liver. Chronic GVHD is ranked based on its severity: stage/grade 1 is mild; stage/grade 4 is severe. Chronic GVHD develops three months or later following transplantation. The symptoms of chronic GVHD are similar to those of acute GVHD, but in addition, chronic GVHD may also affect the mucous glands in the eyes, salivary glands in the mouth, and glands that lubricate the stomach lining and intestines. Any of the populations of immune cells disclosed herein can be utilized. Examples of a transplanted organ include a solid organ transplant, such as kidney, liver, skin, pancreas, lung and/or heart, or a cellular transplant such as islets, hepatocytes, myoblasts, bone marrow, or hematopoietic or other stem cells. The transplant can be a composite transplant, such as tissues of the face. Immune cells can be administered prior to transplantation, concurrently with transplantation, or following transplantation. In some embodiments, the immune cells are administered prior to the transplant, such as at least 1 hour, at least 12 hours, at least 1 day, at least 2 days, at least 3 days, at least 4 days, at least 5 days, at least 6 days, at least 1 week, at least 2 weeks, at least 3 weeks, at least 4 weeks, or at least 1 month prior to the transplant. In one specific, non-limiting example, administration of the therapeutically effective amount of immune cells occurs 3-5 days prior to transplantation.

In some embodiments, the subject can be administered nonmyeloablative lymphodepleting chemotherapy prior to the immune cell therapy. The nonmyeloablative lymphodepleting chemotherapy can be any suitable such therapy, which can be administered by any suitable route. The nonmyeloablative lymphodepleting chemotherapy can comprise, for example, the administration of cyclophosphamide and fludarabine, particularly if the cancer is melanoma, which can be metastatic. An exemplary route of administering cyclophosphamide and fludarabine is intravenously. Likewise, any suitable dose of cyclophosphamide and fludarabine can be administered and is the most common regimen as lymphodepleting chemotherapy before the administration of CAR-T cells or CAR-NK cells. In particular aspects, around 60 mg/kg of cyclophosphamide is administered for two days after which around 25 mg/m² fludarabine is administered for five days.

In certain embodiments, a growth factor that promotes the growth and activation of the immune cells is administered to the subject either concomitantly with the immune cells or subsequently to the immune cells. The immune cell growth factor can be any suitable growth factor that promotes the growth and activation of the immune cells. Examples of suitable immune cell growth factors include interleukin (IL)-2, IL-7, IL-15, and IL-12, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Therapeutically effective doses of immune cells can be administered by a number of routes, including parenteral administration, for example, intravenous, intraperitoneal, intramuscular, intrasternal, or intraarticular injection, or infusion.

The therapeutically effective dose of immune cells for use in adoptive cell therapy is that amount that achieves a desired effect in a subject being treated. For instance, this can be the dose of immune cells necessary to inhibit advancement, or to cause regression of an autoimmune or alloimmune disease, or which is capable of relieving symptoms caused by an autoimmune disease, such as pain and inflammation. It can be the amount necessary to relieve symptoms associated with inflammation, such as pain, edema and elevated temperature. It can also be the amount necessary to diminish or prevent rejection of a transplanted organ.

The immune cell population can be administered in treatment regimens consistent with the disease, for example a single or a few doses over one to several days to ameliorate a disease state or periodic doses over an extended time to inhibit disease progression and prevent disease recurrence. The precise dose to be employed in the formulation will also depend on the route of administration, and the seriousness of the disease or disorder, and should be decided according to the judgment of the practitioner and each patient's circumstances. The therapeutically effective dose of immune cells will be dependent on the subject being treated, the severity and type of the affliction, and the manner of administration. In some embodiments, doses that could be used in the treatment of human subjects range from at least 3.8×10⁴, at least 3.8×10⁵, at least 3.8×10⁶, at least 3.8×10⁷, at least 3.8×10⁸, at least 3.8×10⁹, or at least 3.8×10¹⁰ immune cells/m². In a certain embodiment, the dose used in the treatment of human subjects ranges from about 3.8×10⁹ to about 3.8×10¹⁰ immune cells/m². In additional embodiments, a therapeutically effective amount of immune cells can vary from about 5×10⁶ cells per kg body weight to about 7.5×10⁸ cells per kg body weight, such as about 2×10⁷ cells to about 5×10⁸ cells per kg body weight, or about 5×10⁷ cells to about 2×10⁸ cells per kg body weight. The exact amount of immune cells is readily determined by one of skill in the art based on the age, weight, sex, and physiological condition of the subject. Effective doses can be extrapolated from dose-response curves derived from in vitro or animal model test systems.

The immune cells may be administered in combination with one or more other therapeutic agents for the treatment of the immune-mediated disorder. Combination therapies can include, but are not limited to, one or more anti-microbial agents (for example, antibiotics, anti-viral agents and anti-fungal agents), anti-tumor agents (for example, fluorouracil, methotrexate, paclitaxel, fludarabine, etoposide, doxorubicin, or vincristine), immune-depleting agents (for example, fludarabine, etoposide, doxorubicin, or vincristine), immunosuppressive agents (for example, azathioprine, or glucocorticoids, such as dexamethasone or prednisone), anti-inflammatory agents (for example, glucocorticoids such as hydrocortisone, dexamethasone or prednisone, or non-steroidal anti-inflammatory agents such as acetylsalicylic acid, ibuprofen or naproxen sodium), cytokines (for example, interleukin-10 or transforming growth factor-beta), hormones (for example, estrogen), or a vaccine. In addition, immunosuppressive or tolerogenic agents including but not limited to calcineurin inhibitors (e.g., cyclosporin and tacrolimus); mTOR inhibitors (e.g., Rapamycin); mycophenolate mofetil, antibodies (e.g., recognizing CD3, CD4, CD40, CD154, CD45, IVIG, or B cells); chemotherapeutic agents (e.g., Methotrexate, Treosulfan, Busulfan); irradiation; or chemokines, interleukins or their inhibitors (e.g., BAFF, IL-2, anti-IL-2R, IL-4, JAK kinase inhibitors) can be administered. Such additional pharmaceutical agents can be administered before, during, or after administration of the immune cells, depending on the desired effect. This administration of the cells and the agent can be by the same route or by different routes, and either at the same site or at a different site.

### V. Pharmaceutical Compositions

Also provided herein are pharmaceutical compositions and formulations comprising cells that were subject to cryopreservation, such as immune cells (e.g., T cells or NK cells) and a pharmaceutically acceptable carrier.

Pharmaceutical compositions and formulations as described herein can be prepared by mixing the active ingredients (such as cells) having the desired degree of purity with one or more optional pharmaceutically acceptable carriers (Remington's Pharmaceutical Sciences 22^{nd} edition, 2012), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

### VI. Combination Therapies

In certain embodiments, the compositions and methods of the present embodiments involve a previously cryopreserved cell population in combination with at least one additional therapy. The additional therapy may be radiation therapy, surgery (e.g., lumpectomy and a mastectomy), chemotherapy, gene therapy, DNA therapy, viral therapy, RNA therapy, immunotherapy, bone marrow transplantation, nanotherapy, monoclonal antibody therapy, or a combination of the foregoing. The additional therapy may be in the form of adjuvant or neoadjuvant therapy.

In some embodiments, the additional therapy is the administration of small molecule enzymatic inhibitor or anti-metastatic agent. In some embodiments, the additional therapy is the administration of side-effect limiting agents (e.g., agents intended to lessen the occurrence and/or severity of side effects of treatment, such as anti-nausea agents, etc.). In some embodiments, the additional therapy is radiation therapy. In some embodiments, the additional therapy is surgery. In some embodiments, the additional therapy is a combination of radiation therapy and surgery. In some embodiments, the additional therapy is gamma irradiation. In some embodiments, the additional therapy is therapy targeting PBK/AKT/mTOR pathway, HSP90 inhibitor, tubulin inhibitor, apoptosis inhibitor, and/or chemopreventative agent. The additional therapy may be one or more of the chemotherapeutic agents known in the art.

An immune cell therapy may be administered before, during, after, or in various combinations relative to an additional cancer therapy, such as immune checkpoint therapy. The administrations may be in intervals ranging from concurrently to minutes to days to weeks. In embodiments where the immune cell therapy is provided to a patient separately from an additional therapeutic agent, one would generally ensure that a significant period of time did not expire between the time of each delivery, such that the two compounds would still be able to exert an advantageously combined effect on the patient. In such instances, it is contemplated that one may provide a patient with the antibody therapy and the anti-cancer therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

Various combinations may be employed. For the example below an immune cell therapy is “A” and an anti-cancer therapy is “B”:
A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of any compound or therapy of the present embodiments to a patient will follow general protocols for the administration of such compounds, taking into account the toxicity, if any, of the agents. Therefore, in some embodiments there is a step of monitoring toxicity that is attributable to combination therapy.

A. Chemotherapy

A wide variety of chemotherapeutic agents may be used in accordance with the present embodiments. The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis.

Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards, such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gamma1I and calicheamicin omegaI1); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenisher, such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSKpolysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylornithine (DMFO); retinoids, such as retinoic acid; capecitabine; carboplatin, procarbazine, plicomycin, gemcitabien, navelbine, farnesyl-protein tansferase inhibitors, transplatinum, and pharmaceutically acceptable salts, acids, or derivatives of any of the above.

B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated, such as microwaves, proton beam irradiation (U.S. Pat. Nos. 5,760,395 and 4,870,287), and UV-irradiation. It is most likely that all of these factors affect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

C. Immunotherapy

The skilled artisan will understand that additional immunotherapies may be used in combination or in conjunction with methods of the embodiments. In the context of cancer treatment, immunotherapeutics, generally, rely on the use of immune effector cells and molecules to target and destroy cancer cells. Rituximab (RITUXAN®) is such an example. The immune effector may be, for example, an antibody specific for some marker on the surface of a tumor cell. The antibody alone may serve as an effector of therapy or it may recruit other cells to actually affect cell killing. The antibody also may be conjugated to a drug or toxin (chemotherapeutic, radionuclide, ricin A chain, cholera toxin, pertussis toxin, etc.) and serve as a targeting agent. Alternatively, the effector may be a lymphocyte carrying a surface molecule that interacts, either directly or indirectly, with a tumor cell target. Various effector cells include cytotoxic T cells and NK cells

Antibody-drug conjugates have emerged as a breakthrough approach to the development of cancer therapeutics. Cancer is one of the leading causes of deaths in the world. Antibody-drug conjugates (ADCs) comprise monoclonal antibodies (MAbs) that are covalently linked to cell-killing drugs. This approach combines the high specificity of MAbs against their antigen targets with highly potent cytotoxic drugs, resulting in “armed” MAbs that deliver the payload (drug) to tumor cells with enriched levels of the antigen. Targeted delivery of the drug also minimizes its exposure in normal tissues, resulting in decreased toxicity and improved therapeutic index. The approval of two ADC drugs, ADCETRIS® (brentuximab vedotin) in 2011 and KADCYLA® (trastuzumab emtansine or T-DM1) in 2013 by FDA validated the approach. There are currently more than 30 ADC drug candidates in various stages of clinical trials for cancer treatment (Leal et al., 2014). As antibody engineering and linker-payload optimization are becoming more and more mature, the discovery and development of new ADCs are increasingly dependent on the identification and validation of new targets that are suitable to this approach and the generation of targeting MAbs. Two criteria for ADC targets are upregulated/high levels of expression in tumor cells and robust internalization.

In one aspect of immunotherapy, the tumor cell must bear some marker that is amenable to targeting, i.e., is not present on the majority of other cells. Many tumor markers exist and any of these may be suitable for targeting in the context of the present embodiments. Common tumor markers include CD19, CD20, CA-125, carcinoembryonic antigen, tyrosinase (p97), gp68, TAG-72, HMFG, Sialyl Lewis Antigen, MucA, MucB, PLAP, laminin receptor, erb B, and p155. An alternative aspect of immunotherapy is to combine anticancer effects with immune stimulatory effects. Immune stimulating molecules also exist including: cytokines, such as IL-2, IL-4, IL-12, GM-CSF, gamma-IFN, chemokines, such as MIP-1, MCP-1, IL-8, and growth factors, such as FLT3 ligand.

Examples of immunotherapies currently under investigation or in use are immune adjuvants, e.g., Mycobacterium bovis, Plasmodium falciparum, dinitrochlorobenzene, and aromatic compounds (U.S. Pat. Nos. 5,801,005 and 5,739,169; Hui and Hashimoto, 1998; Christodoulides et al., 1998); cytokine therapy, e.g., interferons α, β, and γ, IL-1, GM-CSF, and TNF (Bukowski et al., 1998; Davidson et al., 1998; Hellstrand et al., 1998); gene therapy, e.g., TNF, IL-1, IL-2, and p53 (Qin et al., 1998; Austin-Ward and Villaseca, 1998; U.S. Pat. Nos. 5,830,880 and 5,846,945); and monoclonal antibodies, e.g., anti-CD20, anti-ganglioside GM2, and anti-p185 (Hollander, 2012; Hanibuchi et al., 1998; U.S. Pat. No. 5,824,311). It is contemplated that one or more anti-cancer therapies may be employed with the antibody therapies described herein.

In some embodiments, the immunotherapy may be an immune checkpoint inhibitor. Immune checkpoints either turn up a signal (e.g., co-stimulatory molecules) or turn down a signal. Inhibitory immune checkpoints that may be targeted by immune checkpoint blockade include adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), programmed death 1 (PD-1), T-cell immunoglobulin domain and mucin domain 3 (TIM-3) and V-domain Ig suppressor of T cell activation (VISTA). In particular, the immune checkpoint inhibitors target the PD-1 axis and/or CTLA-4.

The immune checkpoint inhibitors may be drugs such as small molecules, recombinant forms of ligand or receptors, or, in particular, are antibodies, such as human antibodies (e.g., International Patent Publication WO2015016718; Pardoll, Nat Rev Cancer, 12(4): 252-64, 2012; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example it is known that lambrolizumab is also known under the alternative and equivalent names MK-3475 and pembrolizumab.

In some embodiments, the PD-1 binding antagonist is a molecule that inhibits the binding of PD-1 to its ligand binding partners. In a specific aspect, the PD-1 ligand binding partners are PDL1 and/or PDL2. In another embodiment, a PDL1 binding antagonist is a molecule that inhibits the binding of PDL1 to its binding partners. In a specific aspect, PDL1 binding partners are PD-1 and/or B7-1. In another embodiment, the PDL2 binding antagonist is a molecule that inhibits the binding of PDL2 to its binding partners. In a specific aspect, a PDL2 binding partner is PD-1. The antagonist may be an antibody, an antigen binding fragment thereof, an immunoadhesin, a fusion protein, or oligopeptide. Exemplary antibodies are described in U.S. Pat. Nos. U.S. Pat. Nos. 8,735,553, 8,354,509, and 8,008,449, all incorporated herein by reference. Other PD-1 axis antagonists for use in the methods provided herein are known in the art such as described in U.S. Patent Application No. US20140294898, US2014022021, and US20110008369, all incorporated herein by reference.

In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of nivolumab, pembrolizumab, and CT-011. In some embodiments, the PD-1 binding antagonist is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PD-1 binding antagonist is AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. CT-011, also known as hBAT or hBAT-1, is an anti-PD-1 antibody described in WO2009/101611. AMP-224, also known as B7-DCIg, is a PDL2-Fc fusion soluble receptor described in WO2010/027827 and WO2011/066342.

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to CD80 or CD86 on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to CD80 and CD86, also called B7-1 and B7-2 respectively, on antigen-presenting cells. CTLA4 transmits an inhibitory signal to T cells, whereas CD28 transmits a stimulatory signal. Intracellular CTLA4 is also found in regulatory T cells and may be important to their function. T cell activation through the T cell receptor and CD28 leads to increased expression of CTLA-4, an inhibitory receptor for B7 molecules.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al. (1998) Proc Natl Acad Sci USA 95(17): 10067-10071; Camacho et al. (2004) J Clin Oncology 22(145): Abstract No. 2505 (antibody CP-675206); and Mokyr et al. (1998) Cancer Res 58:5301-5304 can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001014424, WO2000037504, and U.S. Pat. No. 8,017,114; all incorporated herein by reference.

An exemplary anti-CTLA-4 antibody is ipilimumab (also known as 10D1, MDX-010, MDX-101, and Yervoy®) or antigen binding fragments and variants thereof (see, e.g., WO 01/14424). In other embodiments, the antibody comprises the heavy and light chain CDRs or VRs of ipilimumab. Accordingly, in one embodiment, the antibody comprises the CDR1, CDR2, and CDR3 domains of the VH region of ipilimumab, and the CDR1, CDR2 and CDR3 domains of the VL region of ipilimumab. In another embodiment, the antibody competes for binding with and/or binds to the same epitope on CTLA-4 as the above-mentioned antibodies. In another embodiment, the antibody has at least about 90% variable region amino acid sequence identity with the above-mentioned antibodies (e.g., at least about 90%, 95%, or 99% variable region identity with ipilimumab).

Other molecules for modulating CTLA-4 include CTLA-4 ligands and receptors such as described in U.S. Pat. Nos. U.S. Pat. Nos. 5,844,905, 5,885,796 and International Patent Application Nos. WO1995001994 and WO1998042752; all incorporated herein by reference, and immunoadhesins such as described in U.S. Pat. No. 8,329,867, incorporated herein by reference.

D. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

E. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VII. Articles of Manufacture or Kits

An article of manufacture or a kit is provided comprising cryopreservation medium or components thereof and optionally immune cells. The article of manufacture or kit can further comprise a package insert comprising instructions for using the cryopreservation and/or immune cells to treat or delay progression of cancer in an individual or to enhance immune function of an individual having cancer. Any of the cryopreservation media components and optionally antigen-specific immune cells described herein may be included in the article of manufacture or kits. Suitable containers include, for example, bottles, vials, bags and syringes. The container may be formed from a variety of materials such as glass, plastic (such as polyvinyl chloride or polyolefin), or metal alloy (such as stainless steel or hastelloy). In some embodiments, the container holds the formulation and the label on, or associated with, the container may indicate directions for use. The article of manufacture or kit may further include other materials desirable from a commercial and user standpoint, including other buffers, diluents, filters, needles, syringes, and package inserts with instructions for use. In some embodiments, the article of manufacture further includes one or more of another agent (e.g., a chemotherapeutic agent, and anti-neoplastic agent). Suitable containers for the one or more agent include, for example, bottles, vials, bags and syringes.

### VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Cryopreservation of NK-CAR Cells

In specific embodiments, at a suitable time an infusion product comprising NK-CAR cells (as an example of cells) may be harvested, washed and cryopreserved. The cells are harvested at a time of need, such as following a suitable time for expansion of cells. A sample may be removed for cell count and viability, and flow cytometry to characterize the phenotype of the expanded cells, in some cases. Samples may also be removed from the culture for Mycoplasma testing (for example, with PCR and MycoAlert), as one example. A gram stain is often performed to make sure no bacteria are present. If the cell viability is >70%, cells may be collected by centrifugation and a sample may be removed from the supernatant for PCR testing (if deemed necessary) by a cell culture-based assay.

The cell product may be washed, for example with Plasma-Lyte A containing 0.5% Human Serum Albumin (HSA) to remove culture media reagents. Samples are collected for release and non-release testing. The final cell suspension may be prepared for cryopreservation by washing with a mixture of Plasma-Lyte A containing 10% Human AB Serum. The cells are cryopreserved in a mixture of 95% Human AB serum containing 5% DMSO, IL-2 400 U/mL, and IL-21 20 ng/mL. The cells are stored in vapor phase liquid nitrogen until ready for infusion. Release testing includes testing for purity, Gram Stain, Mycoplasma (MycoAlert), Visual Inspection, Viability (7AAD), Immunophenotyping and Endotoxin (LAL). Non-release testing includes Mycoplasma by PCR (Day 15) and Vector Copy Number (VCN) Analysis by QPCR (Day 15).

### Example 2—Preparation of Cryopreserved NK-CAR Cells for Infusion

On the day of infusion, the cryopreserved cells are thawed at 37° C. The cells are washed twice with a mixture of Plasma-Lyte A containing 0.5% HSA. Samples are collected for Cell Count, Immunophenotyping and Viability (7AAD). The cell product is then resuspended at the required dose level in Plasma-Lyte A containing 0.5% HSA. The final infusion volume may be approximately 20 mL for Dose 1, and approximately 100 mL for Dose 2 and Dose 3. Samples are removed from the final infusion product for release testing that includes testing for Visual Inspection, Viability (7AAD), Gram Stain, Immunophenotyping and Cell Count (Cell Dose). Non-release testing includes Sterility Testing (BD Bactec).

### Example 3—Cryopreservation Embodiments

As an example only, cryopreservation methods were utilized for NK cells that were derived from cord blood (CB) and in which case their specificity was redirected by genetically engineering them to express tumor-specific chimeric antigen receptors (CARs) that could enhance their anti-tumor activity without increasing the risk of graft-versus-host disease (GVHD). This allows for providing an ‘off-the-shelf’ source of cells for therapy, such as immunotherapy of any cancer expressing the target.

For cryopreservation, the CB NK cells were suspended in a GMP cryopreservation medium comprising 5% DMSO, 95% Human AB Serum, 400 units IL-2/ml, and 20 ng IL-21/ml, and the cells were frozen in liquid nitrogen using a rate controlled method.

Following the thawing of the cultured CB-NK cells that had been frozen as the final product, the cells were characterized. Post-thaw, the cell viability was more than 80% and the cell recovery was more than 85%. Moreover, the NK cells cryopreserved in FMC exerted significantly better cytotoxicity against K562 and Raji targets compared to NK cells cryopreserved in FM alone (p=0.02 and p=0.0004, respectively).

Thus, the exemplary CAR-transduced cord blood-derived NK cells can provide an off-the-shelf source of personalized NK cells that can recognize and attack many cancers including both liquid and solid tumors. Retroviral transduction of cord blood derived natural killer cells allows for longer persistence and improved efficacy of the engineered cells for use in the immunotherapy of many cancers and potentially for the treatment of many viral infections.

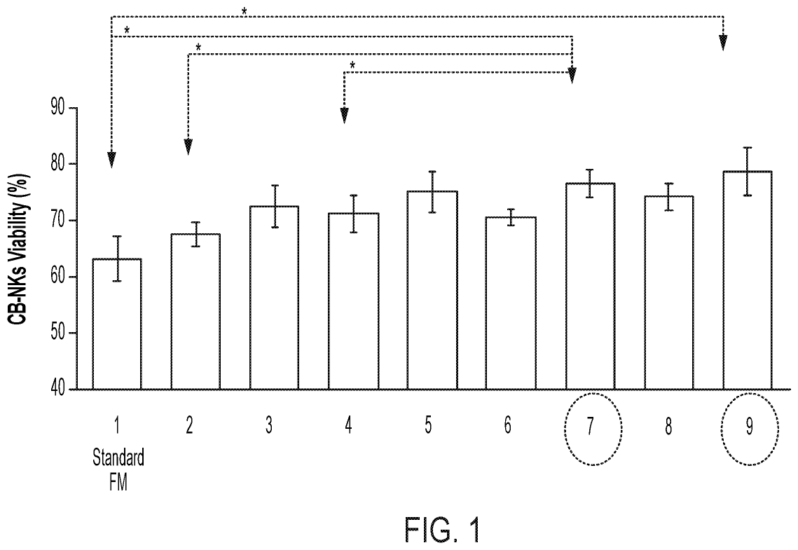

With further studies, the viability of CB-NKs cryopreserved in nine different freezing media comprising different combinations of cytokines was tested, many of which had statistically significant viability than standard freezing media (FIG. 1). Standard freeze media is 95% human AB serum+5% DMSO. In FIG. 1, the combinations of cytokines include the following: (1) Standard freezing media; (2) IL2 alone; (3) IL15 alone; (4) IL21 alone; (5) IL2+IL21; (6) IL2+IL15; (7) IL21+IL15; (8) IL2+IL15+IL21; and (9) research grade freeze media (Sigma).

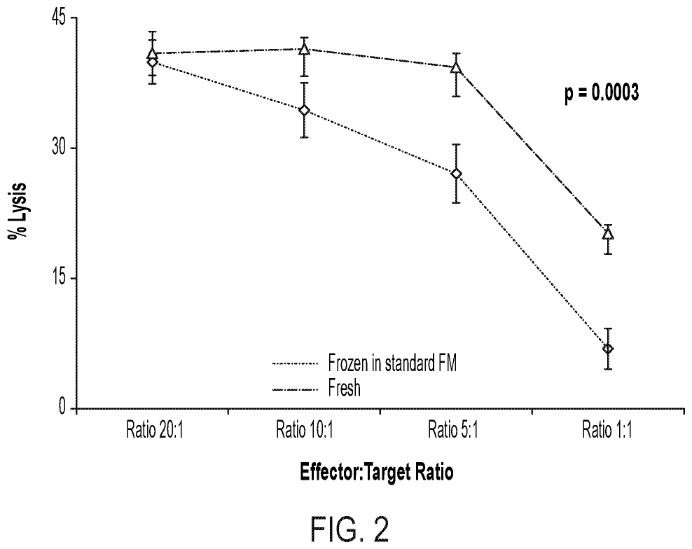

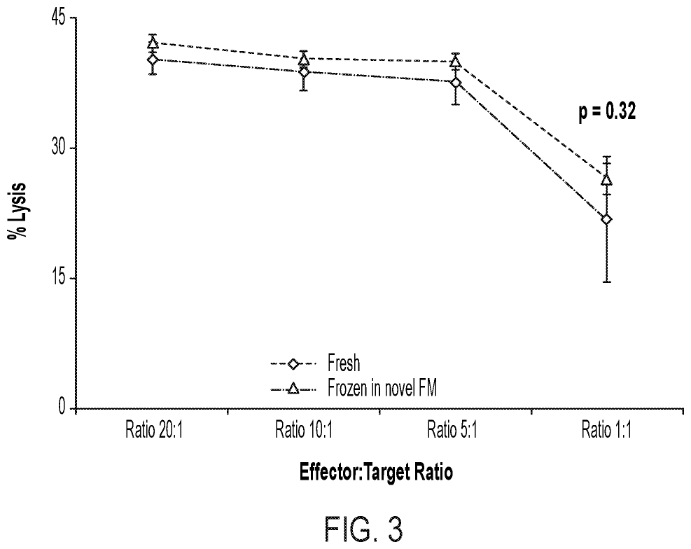

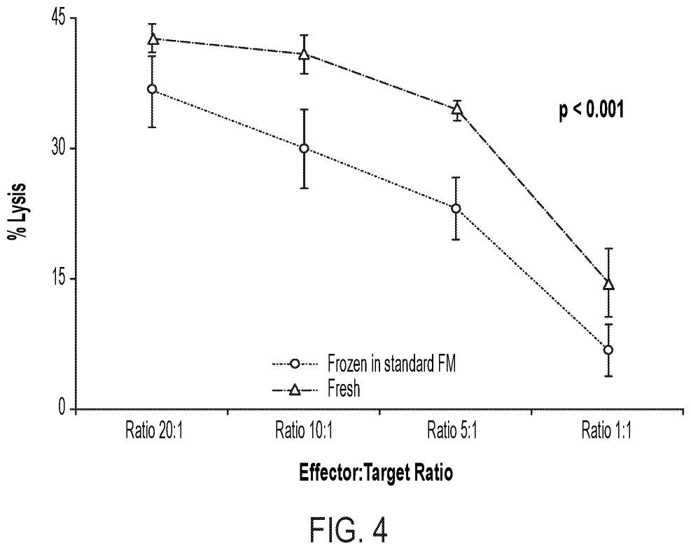

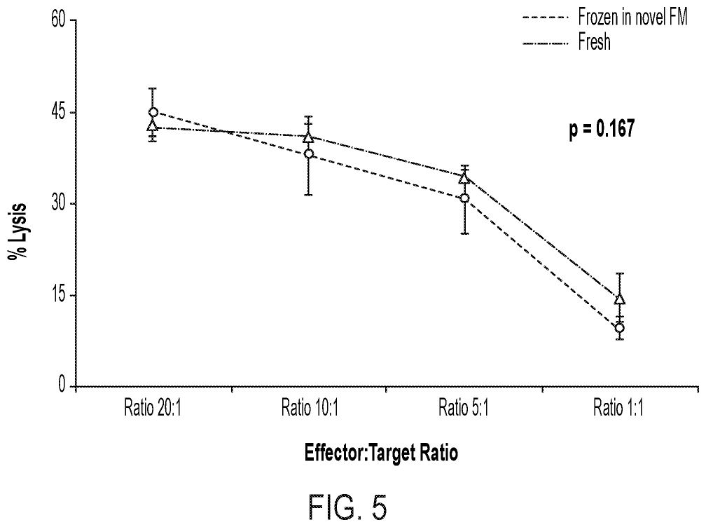

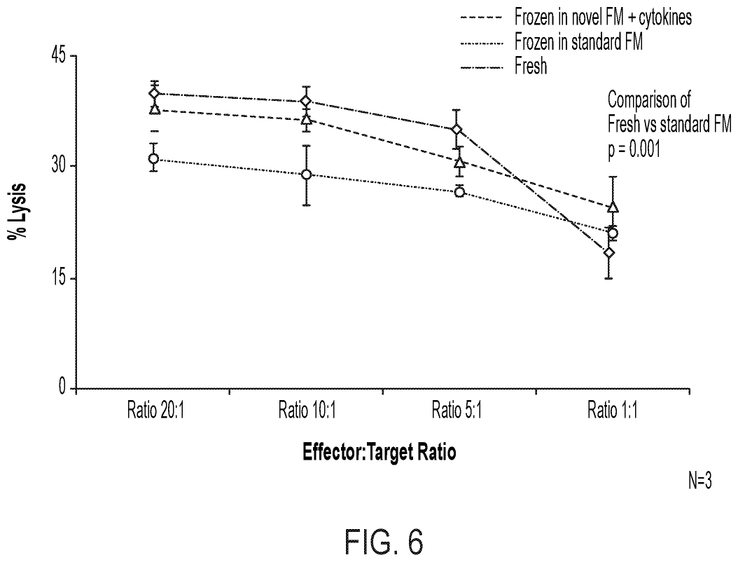

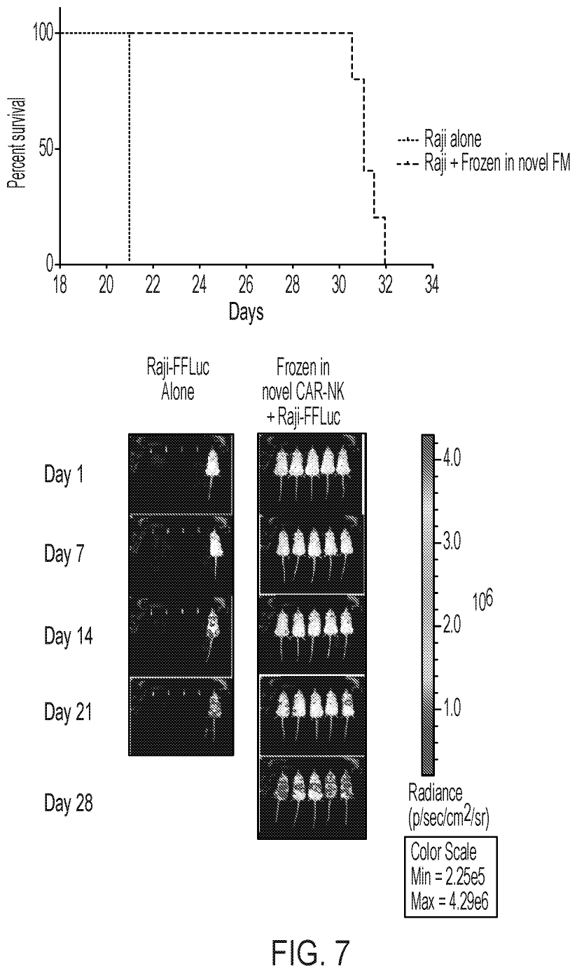

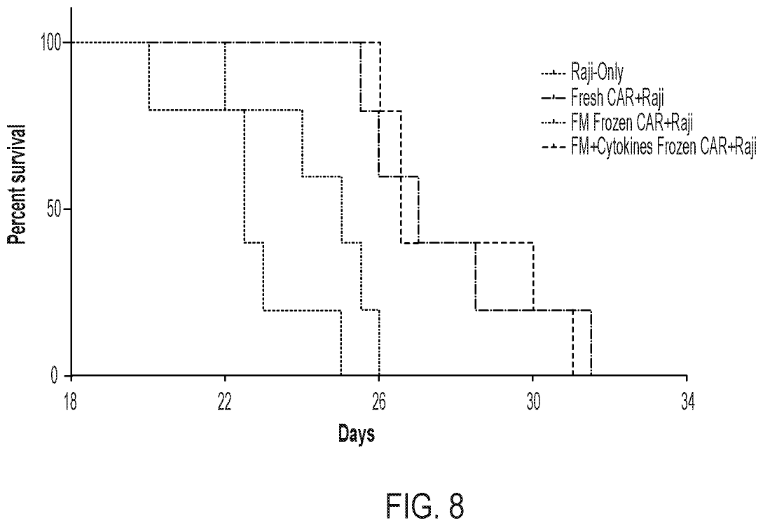

FIG. 2 shows a comparison of NK cells cryopreserved in GMP standard freeze media with fresh NK cells. The NK cells cryopreserved in GMP freeze media exert inferior cytotoxicity against K562 targets post-thaw compared to fresh NK cells. In contrast, NK cells cryopreserved in GMP freeze media and cytokines exert similar cytotoxicity against K562 targets post-thaw compared to fresh NK cells (FIG. 3). Testing NK cells that express a chimeric antigen receptor (CAR), FIG. 4 shows that CAR-expressing NK cells cryopreserved in GMP standard freezing media exert inferior cytotoxicity against Raji targets post-thaw compared to fresh CAR-expressing NK cells. However, CAR-expressing NK cells cryopreserved in GMP freeze media and cytokines exert similar cytotoxicity against Raji targets post-thaw compared to fresh CAR-expressing NK cells (n=3). Analogously, CAR-expressing NK cells cryopreserved in GMP standard freezing media and cytokines exert similar cytotoxicity against Raji targets post-thaw compared to fresh CAR-expressing NK cells, and they are superior to CAR-expressing NK cells frozen in standard GMP freeze media (FIG. 6). CAR-expressing NK cells frozen in the cryopreservation media of the disclosure including cytokines and infused immediately post-thaw in Raji-engrafted mice exert disease control. FIG. 8 shows that CAR-expressing NK cells frozen in cryopreservation media of the disclosure including cytokines (FM+cytokines) and infused immediately post-thaw in Raji-engrafted mice exert similar disease control as fresh CAR-expressing NK cells, and they are superior to CAR-expressing NK cells frozen in standard GMP freeze media (FM).

### Example 4—Robust, Cryopreservation of GMP-Compliant CAR-NK Cell Products for Off-the-Shelf Immunotherapy

The present example concerns production of frozen cell products that may be utilized as “off-the-shelf” cell therapy that can be thawed and infused into individuals in need thereof with no delay needed for production. The methods and compositions may be applied to any type of cell, including NK cells, such as umbilical cord blood-derived natural killer (CB-NK) cells. The cells may be transduced with one or more types of vectors, including viral vectors such as retroviral vectors. In specific embodiments, the vectors produce gene products in the cells that allow the cells to target cancer, such as through cancer antigens. Specific examples of targets include CD19+ lymphoid cancers, myeloid tumor, and solid tumor cancer antigens.

In certain embodiments, the disclosure is particularly suited for peripheral blood derived NK cells that under normal circumstances do not allow for an ‘off-the-shelf’ approach. This is because a donor has to be identified for NK cell donation in each case. In specific embodiments, chimeric antigen receptor (CAR)-engineered NK cells are particularly suited for methods and compositions of the disclosure. Although CAR-engineered NK92 cells known in the art, NK92 is an NK cell line derived from a lymphoma patient, which lacks many of the NK cell receptors important for NK cell cytotoxicity. Because the cell line is derived from a patient with lymphoma, it must be irradiated prior to infusion or there is a risk it will cause lymphoma in the recipient. The radiation will significantly reduce their ability to proliferate and persist. These cells are therefore likely to be less effective than CAR-modified CB-NK cells that express the full array of NK cell receptors.

The present disclosure provides an off-the-shelf source of cells for immunotherapy of cancer cells. The main advantage over CAR-T cells is that NK cells are much less likely to cause graft-versus-host disease (GVHD), while off-the-shelf CAR T cells, in the absence of full HLA-matching, if infused into a patient are likely to cause lethal GVHD. An advantage over peripheral blood-derived CAR-NK cells is the availability of CB banks worldwide, which would allow off-the-shelf sources of CAR-engineered cord blood derived NK cells for immunotherapy without the need to recruit donors for NK cell collection. CAR engineered NK cells are more likely to be effective than NK92-CAR transduced cells, as the latter does not possess the full machinery of NK cell killing compared to the former and needs to be irradiated prior to infusion, thus negatively impacting their persistence and proliferation. Moreover, the ability to cryopreserve NK cells such that post thaw they retain the same potency as their fresh counterpart is extremely valuable, as it will allow for this type of immunotherapy to be used as an off-the-shelf therapy for patients with cancer.

Particular embodiments for the methods and compositions include at least the following: the robust expansion of NK cells from frozen/thawed CB units in co-cultures comprising universal antigen presenting cells (uAPCs) or other feeder cells and cytokines including interleukin (IL)-2; the high and consistent transduction levels of the NK cells with the CAR constructs; the rapid production of the highly potent CB—NK-CAR cells that can be infused fresh or frozen for subsequent infusion. The generated frozen NK-CAR products will provide truly “off-the-shelf” cell therapy that can be thawed and infused into patients at will and with no need to postpone treatment while waiting for production of the cells.

In one example, NK cells isolated from umbilical cord blood (CB) of healthy donors are co-cultured with antigen presenting cells (APC) such as K562-based feeders or other feeder cells (such as lymphoblastoid cells lines or beads), and this is done in the presence of one or more cytokines, including IL-2. The NK cells are then transduced with retroviral or lentiviral vectors (as examples), or electroporated with sleeping beauty or piggy-back constructs, and these constructs or vectors allow the cells to target hematologic and solid cancers. The transduced cells are then further expanded in co-cultures with the APCs or other feeders and IL-2 (or other cytokine(s)) to obtain the potent CB-NK cells. In one specific case the NK cells are CAR-transduced NK cells. Those cells can be infused fresh or can be frozen in media comprising cytokines for thaw and infusion at a later date. A same or similar approach can be used to generate and cryopreserve CAR-transduced NK cells from the peripheral blood (PB) of healthy donors or from PB of patients, from NK cell lines such as NK92 cells, from induced pluripotent stem cells, or hematopoietic stem cells or from bone marrow. The procedures for generating the desired NK cells (such as genetically modified CB-NK cells with retroviral vectors) is as follows:

CAR-NK Cell Cryopreservation. In a comprehensive series of studies the inventors have optimized the cryopreservation of CAR-NK cells. The addition of dextran and albumin (extracellular cryoprotectants) and DMSO (intracellular cryoprotectant) was shown to preserve and even improve the cytotoxicity of CAR NK cells post-thaw against tumor cells compared to standard cryopreservation techniques.

The inventors compared different concentrations of PlasmaLyte, extracellular cryoprotectants (dextran and human albumin) and intracellular cryoprotectant concentrations (DMSO 5% vs 7.5%)+/−cytokines (IL-2 or IL15 alone, or combinations of IL-2/IL-15 or IL-2/IL-21) using viability and in vitro killing assays.

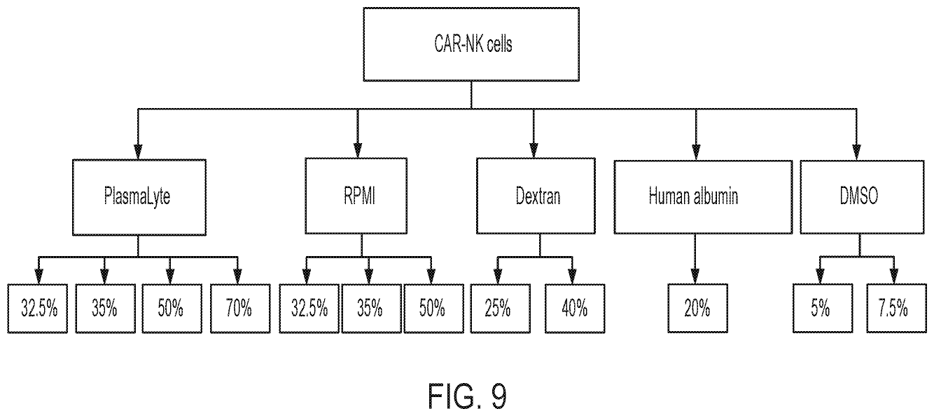

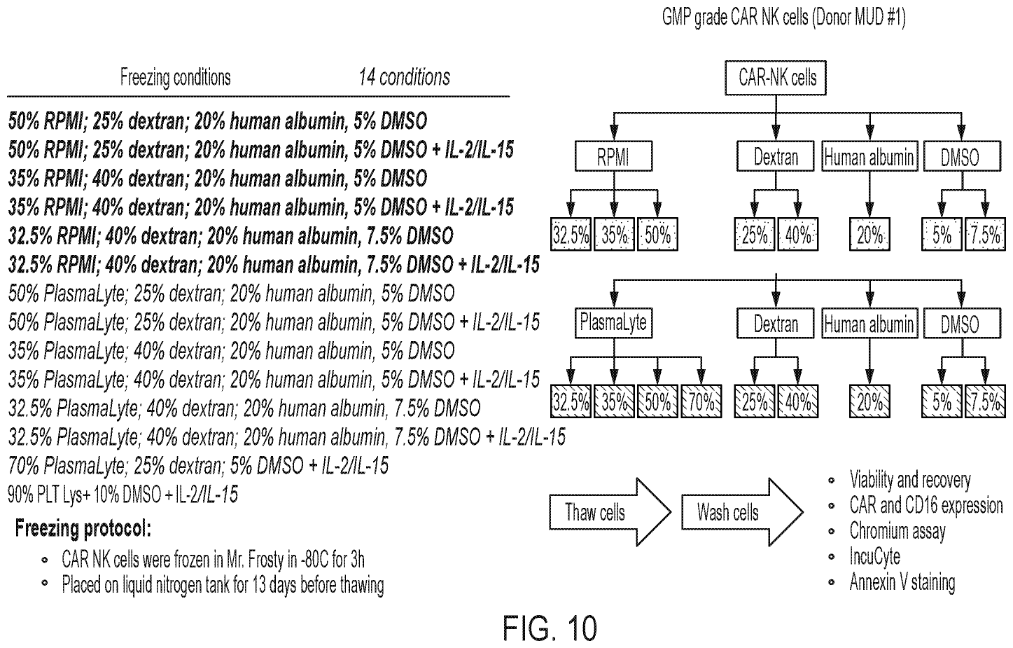

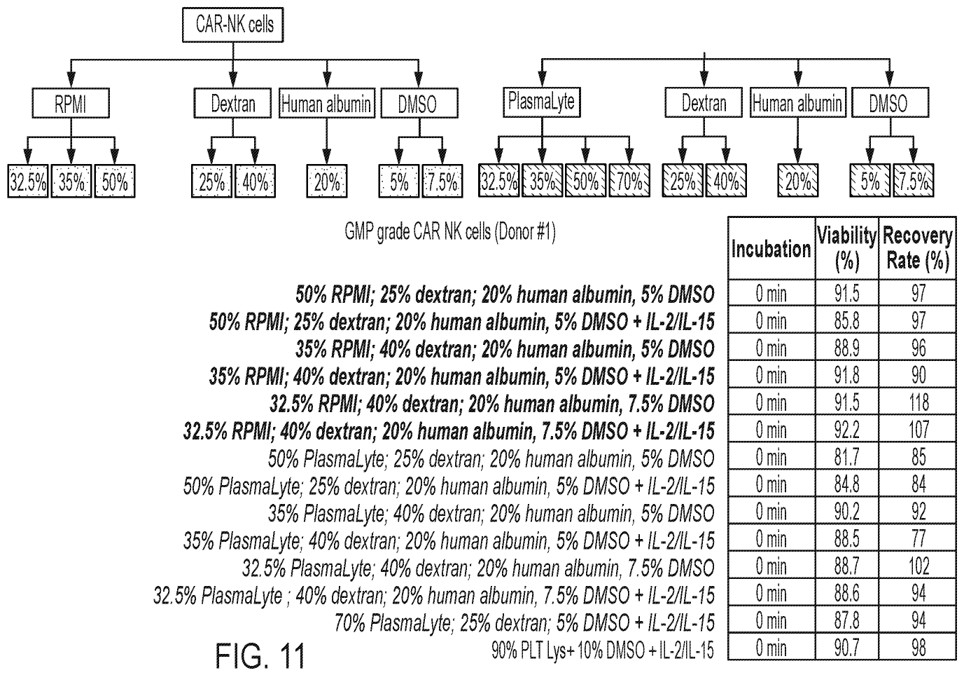

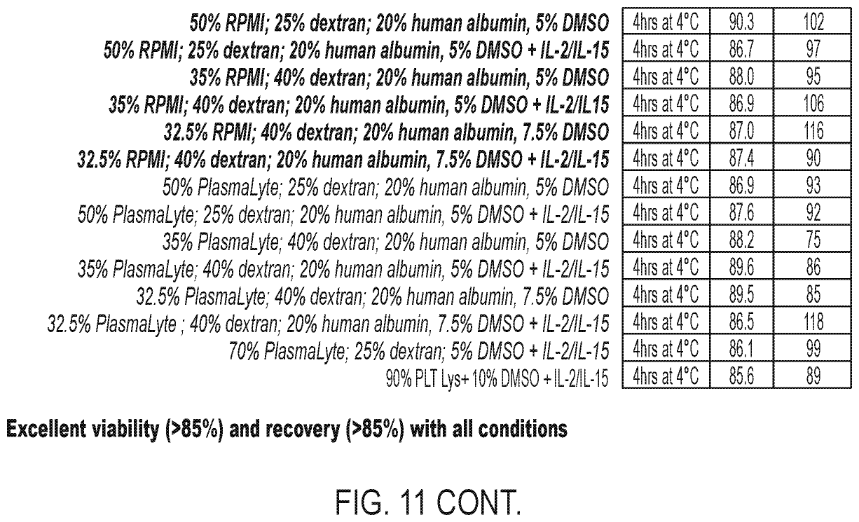

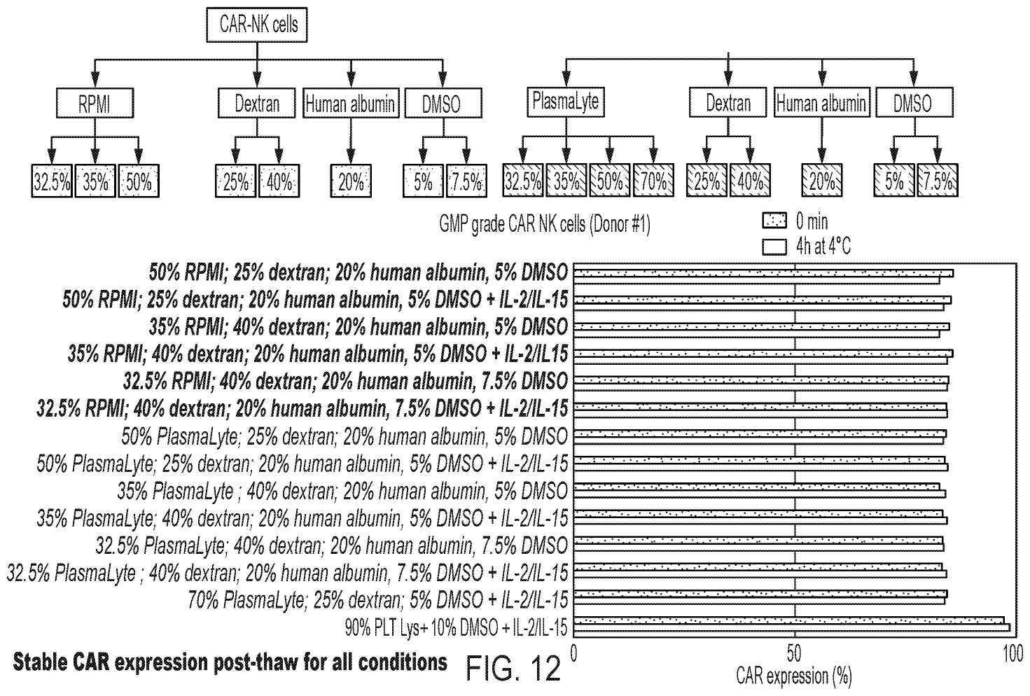

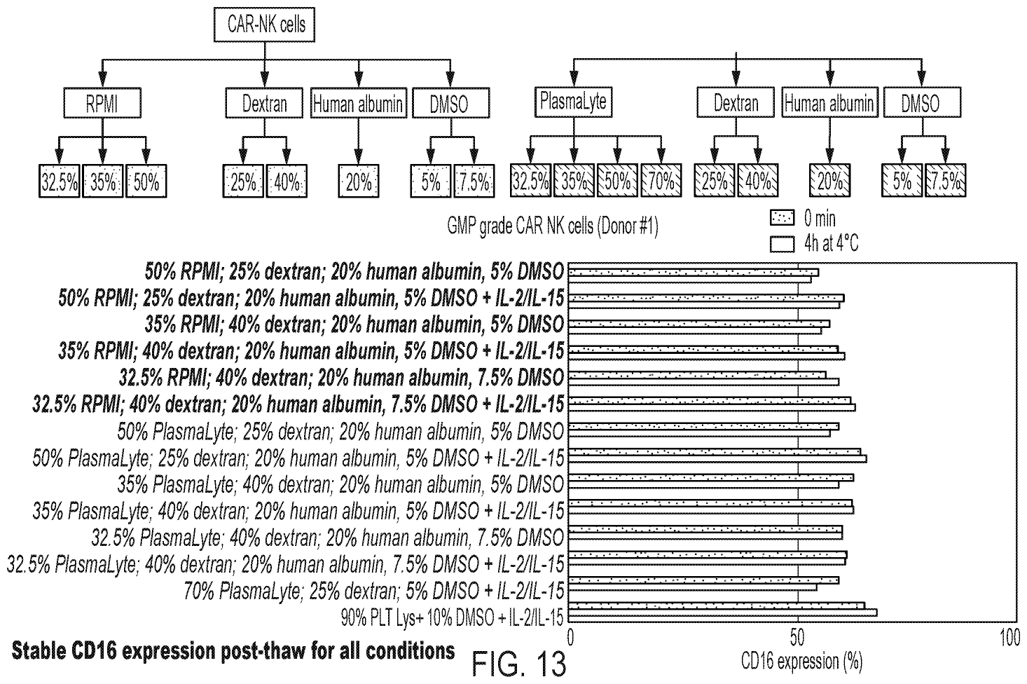

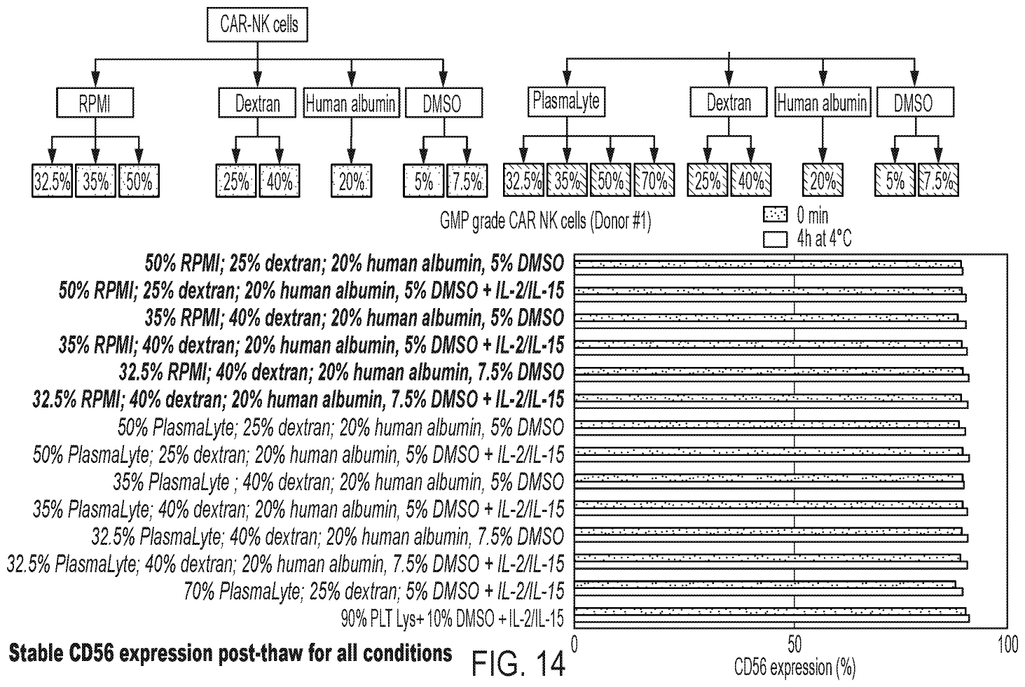

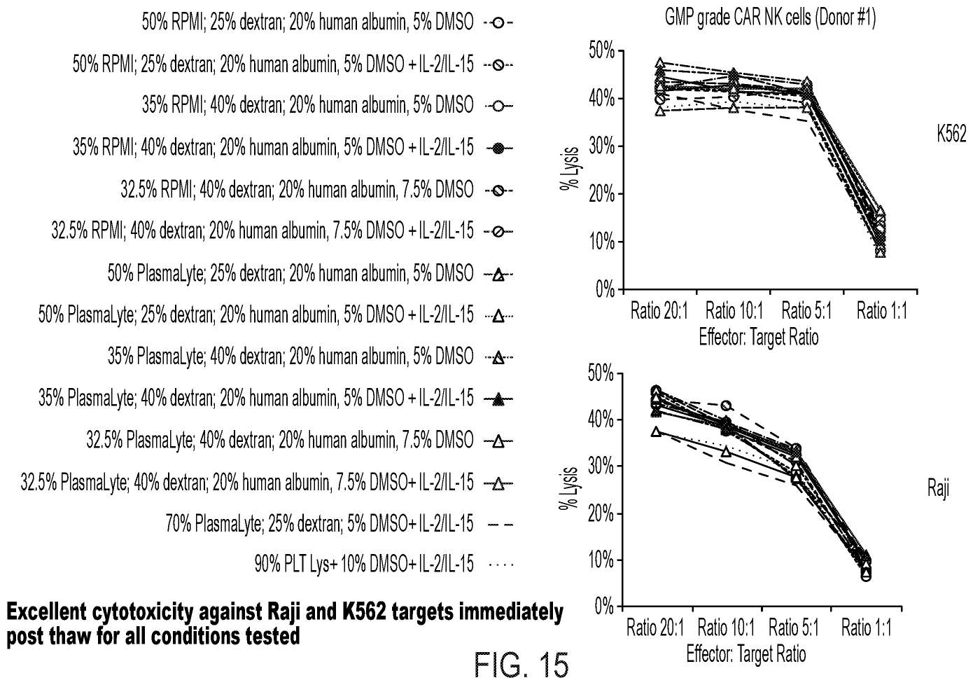

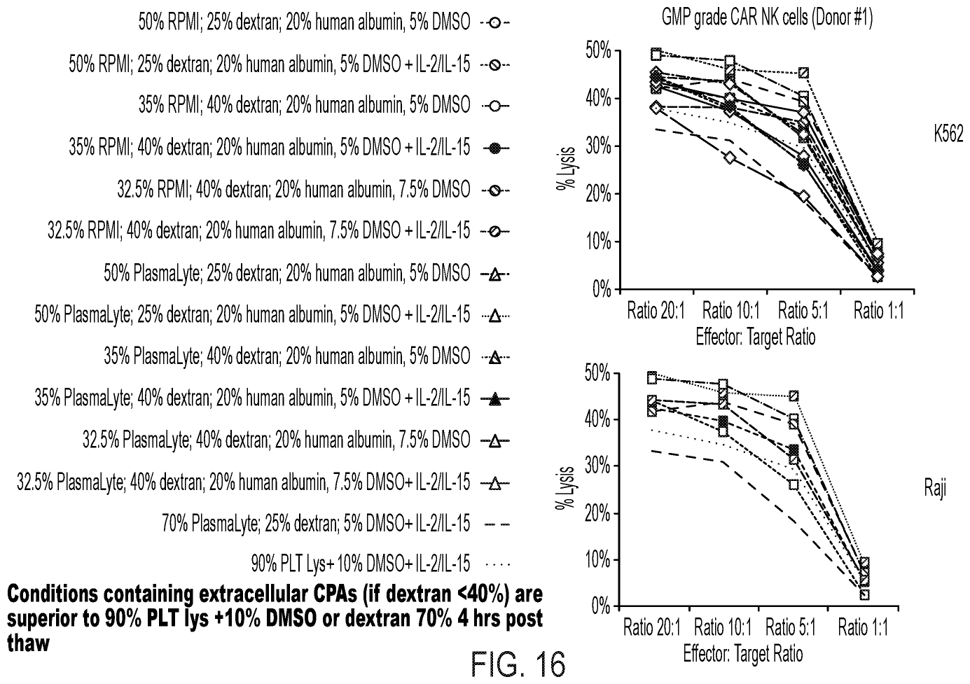

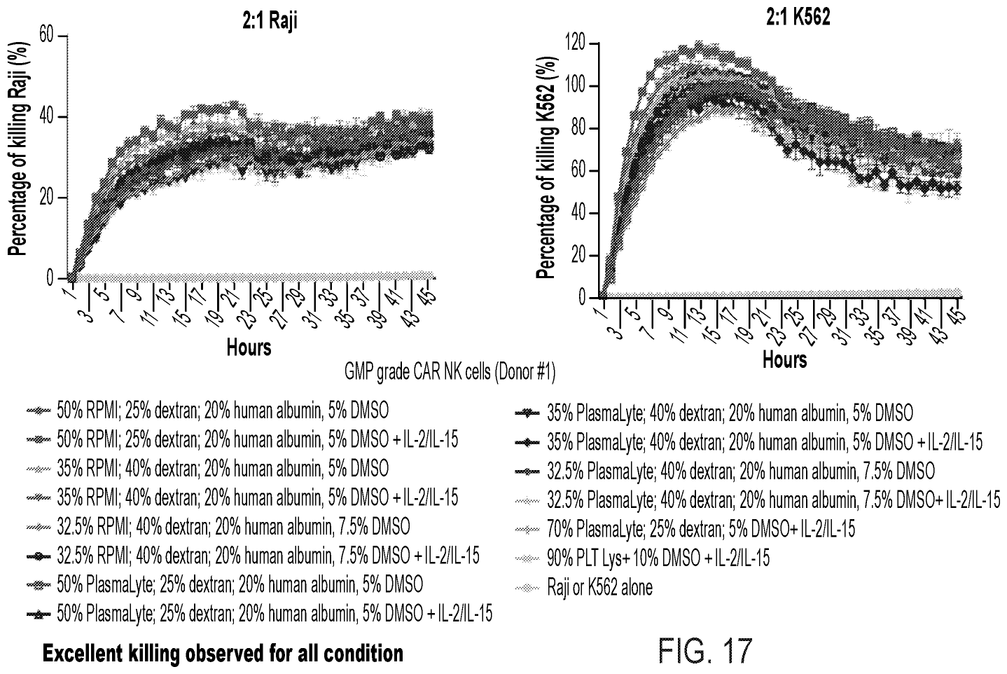

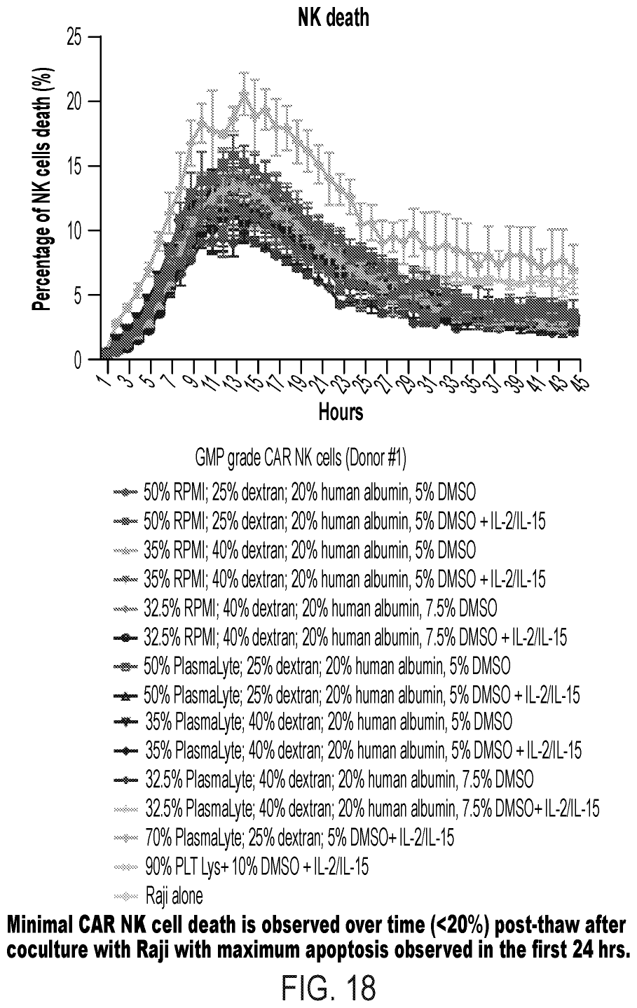

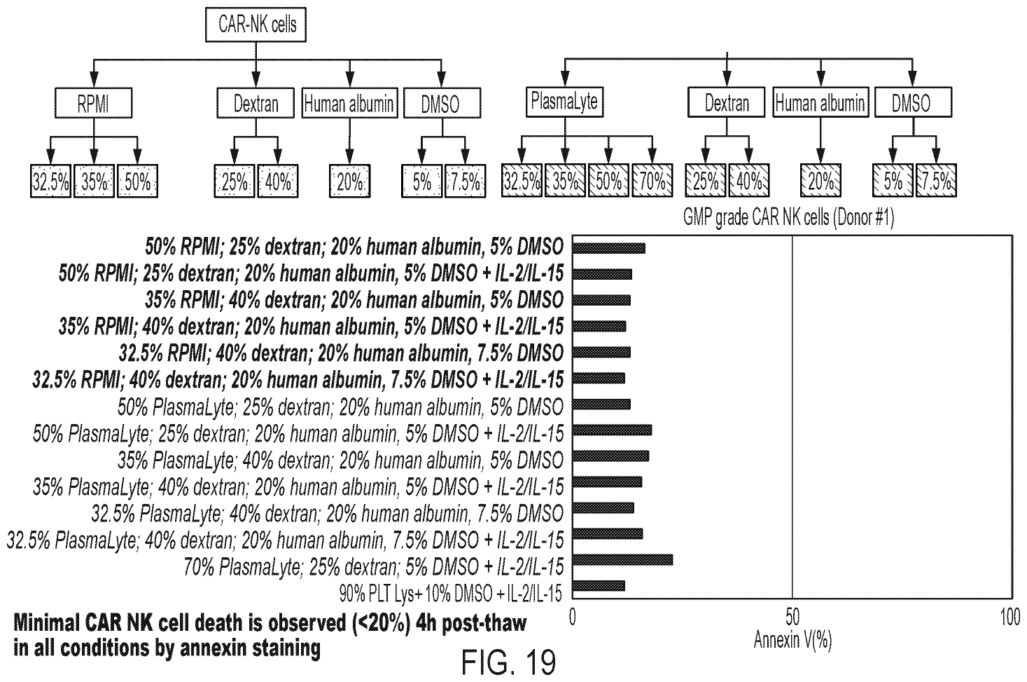

An example of a study design is shown in FIG. 9, where the CAR-NK cells were frozen in the freezing media comprising various concentrations of PlasmaLyte, RPMI, dextran, human albumin and DMSO. Additional experimental detail is summarized in FIG. 10, including the comparison of RPMI vs PlasmaLyte, different extracellular cryoprotectants (dextran and human albumin), the addition of cytokines to the freezing media (IL-2/IL-15) and comparing different intracellular cryoprotectant concentrations (DMSO 5% vs 7.5%). The post-thaw viabilities and recoveries of the CAR-NK cells immediately or 4 hours post-thaw are shown FIG. 11, demonstrating no major differences among these conditions. FIG. 12 shows the post-thaw CAR-expression that was not significantly different among the various conditions. FIG. 13 shows the post-thaw CD16-expression that was not significantly different among the various conditions. FIG. 14 shows the post thaw CD56-expression which was not significantly different among the various conditions. FIG. 15 shows that excellent cytotoxicity was demonstrated against Raji and K562 targets immediately post thaw for all conditions tested. In FIG. 16, it was demonstrated that the optimal concentration of the extracellular cyto-protective agents (CPAs), dextran is 40% or less and that CAR NK cells frozen in conditions containing 40% dextran or less have superior cytotoxicity against K562 and Raji targets when compared to those frozen in 90% platelet (PLT) lysate+10% DMSO+IL2/IL-15 or dextran 70% 4 hrs post thaw. FIG. 17 showed excellent killing of the Raji and K562 targets in the IncuCyte assay for all conditions tested. Again using the IncuCyte assay in FIG. 18, the inventors demonstrated minimal CAR NK cell death observed over time (<20%) post-thaw after coculture with Raji with maximum apoptosis observed in the first 24 hrs. FIG. 19 shows minimal CAR NK cell death (<20%) 4 h post-thaw for all conditions by annexin staining.

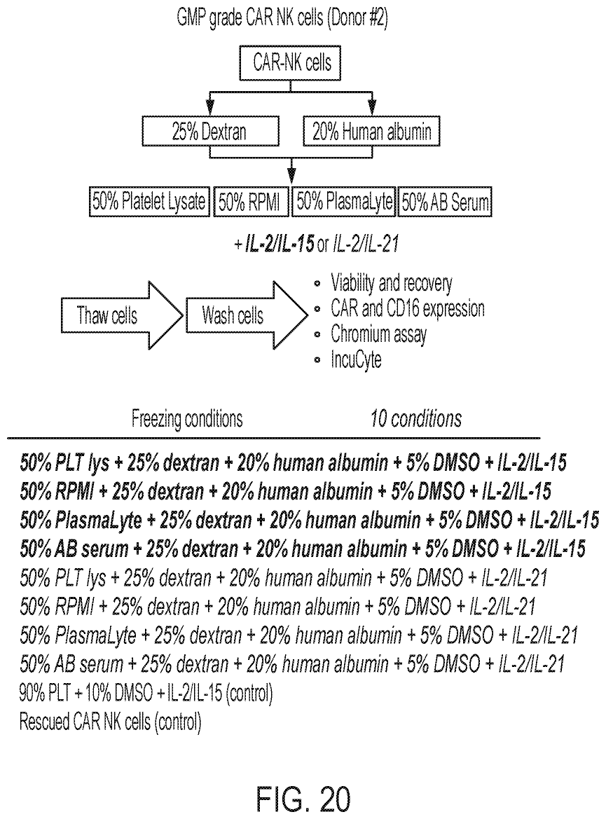

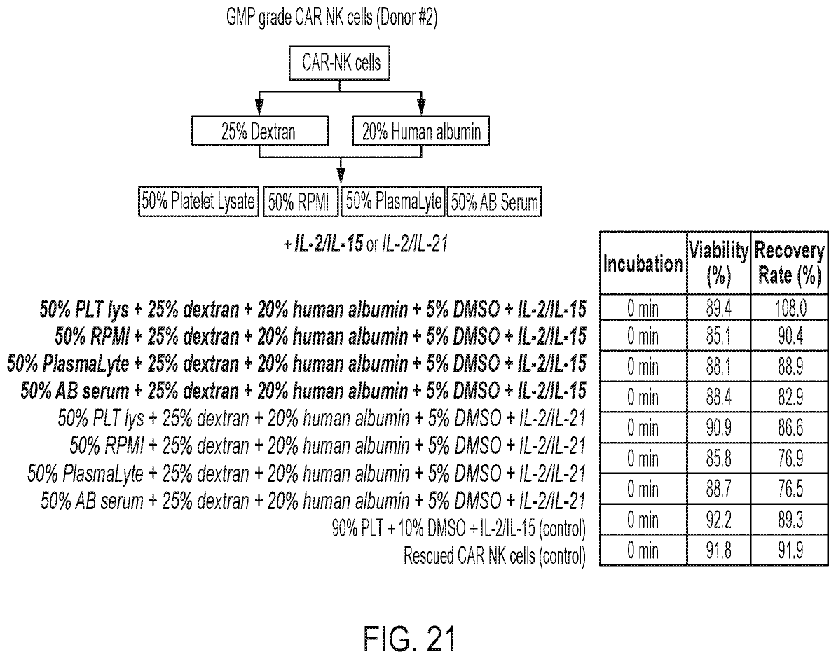

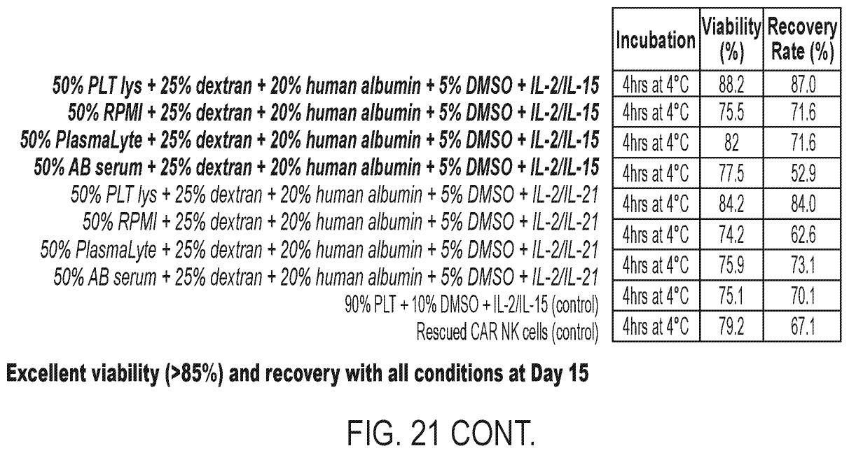

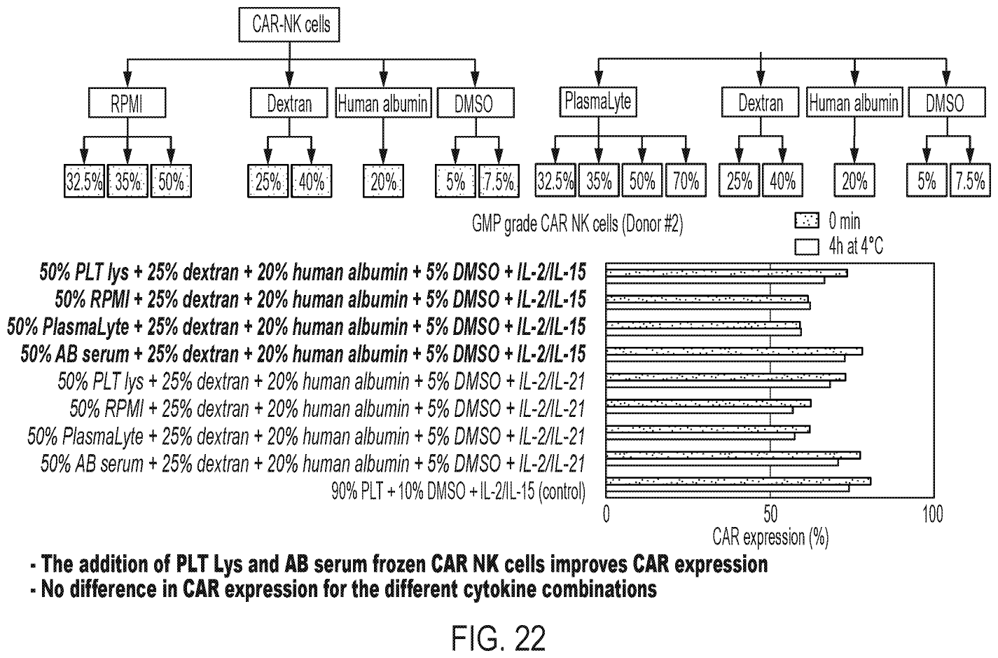

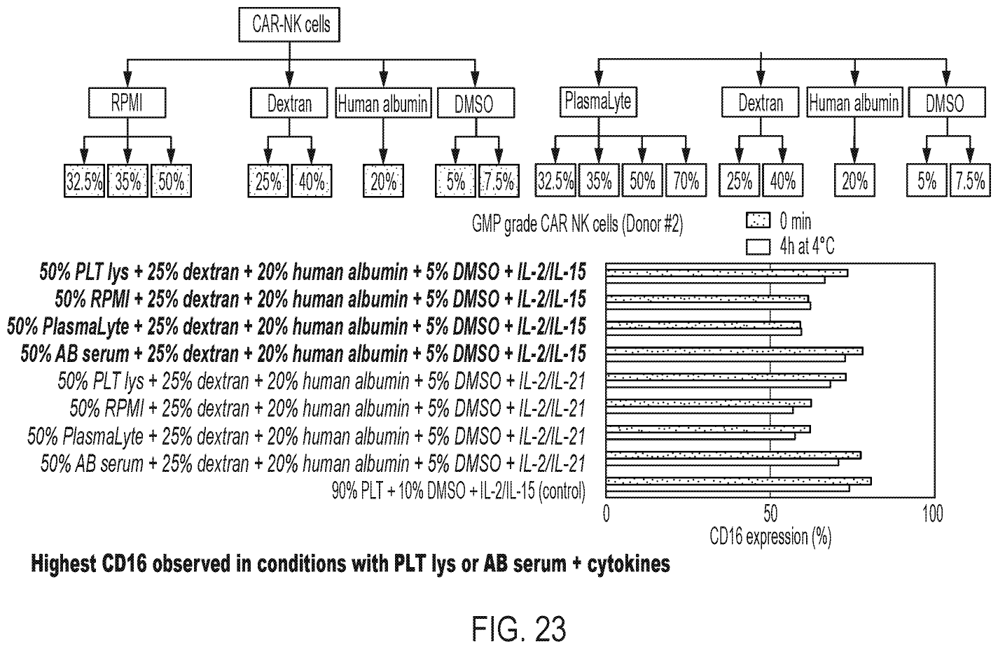

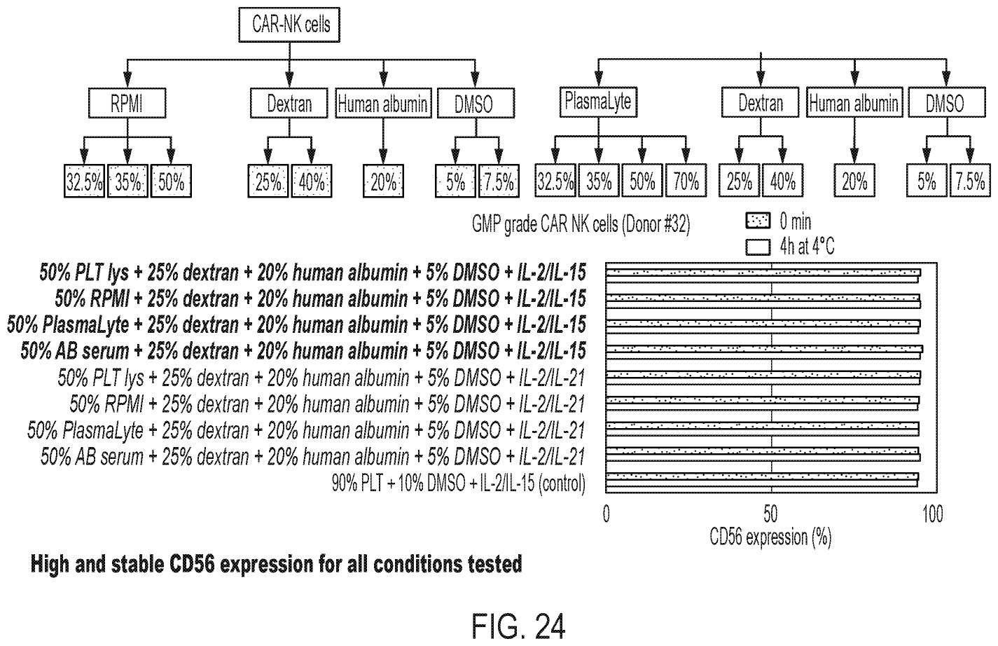

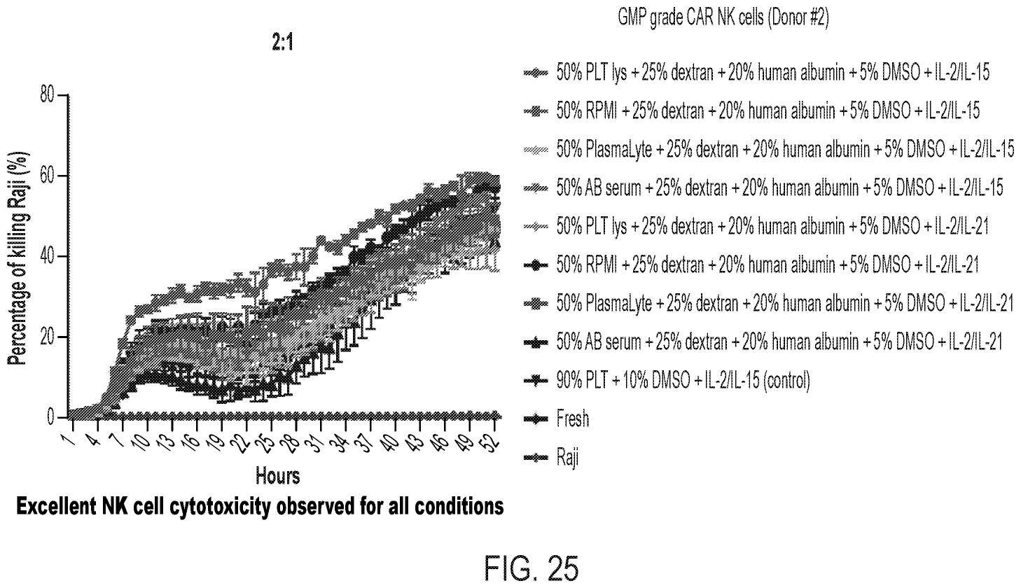

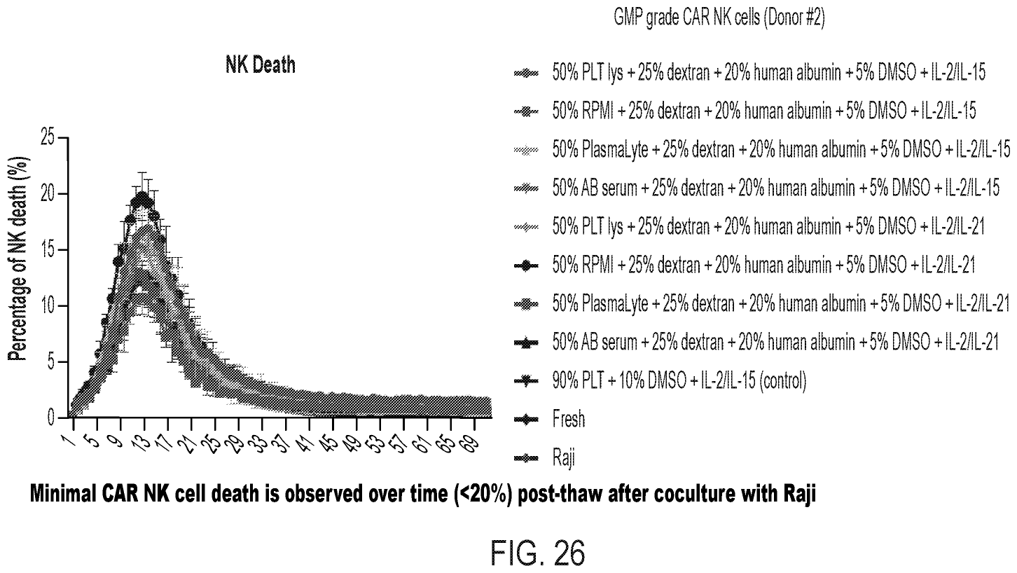

The impact was next tested of adding platelet lysate (PLT Lys) or AB serum to the extra and intracellular CPAs in Plasmalyte vs RMPI and with different cytokine combinations (IL-2/IL-15 vs IL-2/IL-21). CAR NK cells were expanded for either 15 days or 22 days prior to cryopreservation. The more detailed experimental design is shown in FIG. 20 describing evaluation of the freezing conditions for CAR NK cells that were expanded for 15 vs. 22 days. FIG. 21 demonstrated the excellent viability (>85%) and recovery post thaw for CAR NK cells expanded for 15 days and cryopreserved using the different conditions. FIG. 22 demonstrated that the addition of PLTLysate and AB serum to the freeze media preserved CAR expression post thaw, with no difference in CAR expression observed with different cytokine combination (IL-2/IL-15 vs IL-2/IL-21). FIG. 23 demonstrated the highest CD16 observed in conditions where PLT lysate or AB serum plus cytokines were added to the freeze media. FIG. 24 demonstrated high and stable CD56 expression for all conditions tested. FIG. 25 shows that CAR NK cells expanded for 22 days and cryopreserved using the different conditions retain excellent cytotoxicity against Raji cells immediately post thaw in the IncuCyte assay. FIG. 26 shows minimal CAR NK cell death observed in the IncuCyte assay over time (<20%) post-thaw after coculture with Raji.

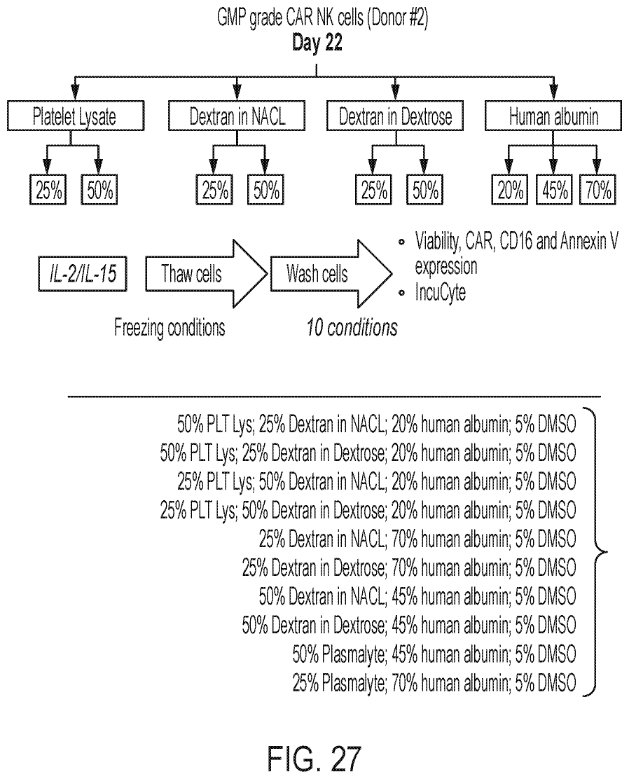

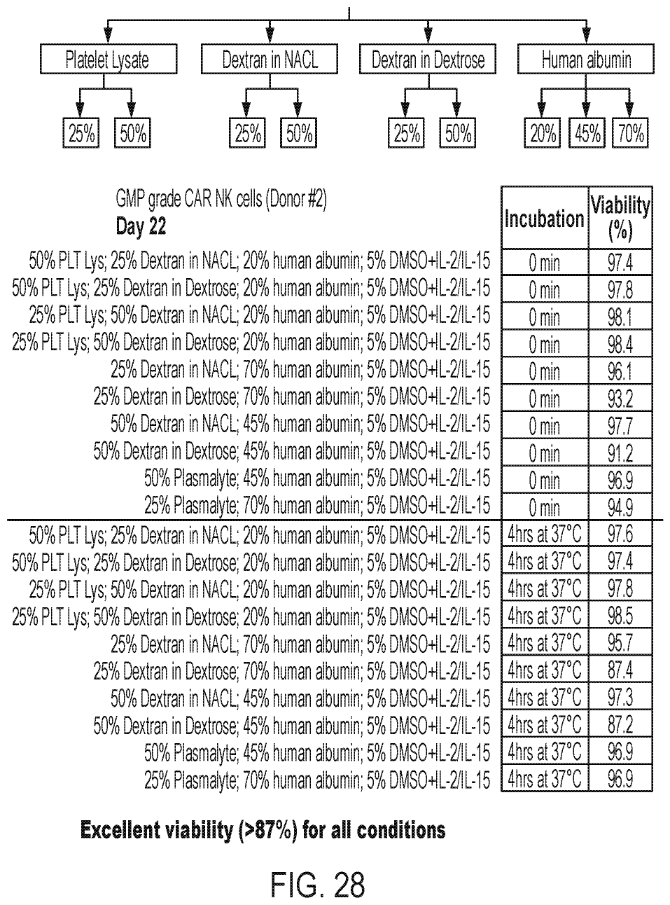

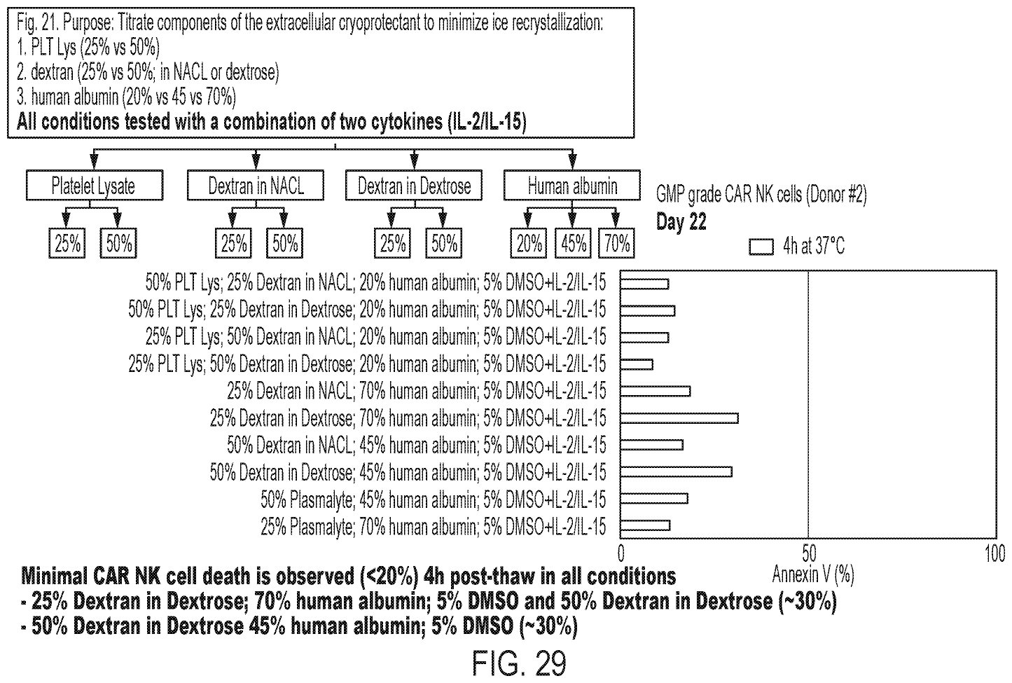

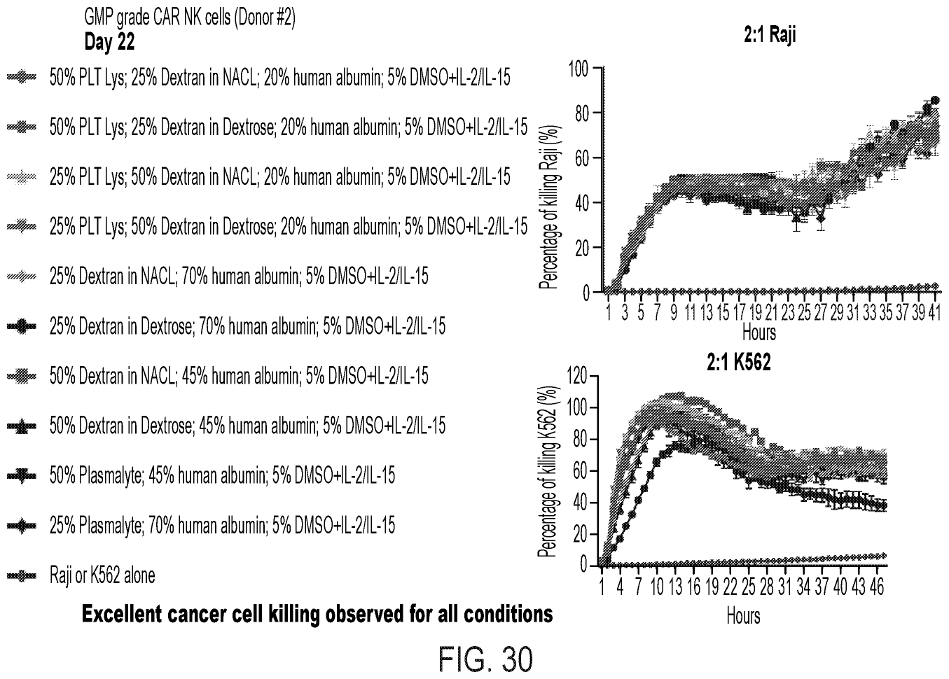

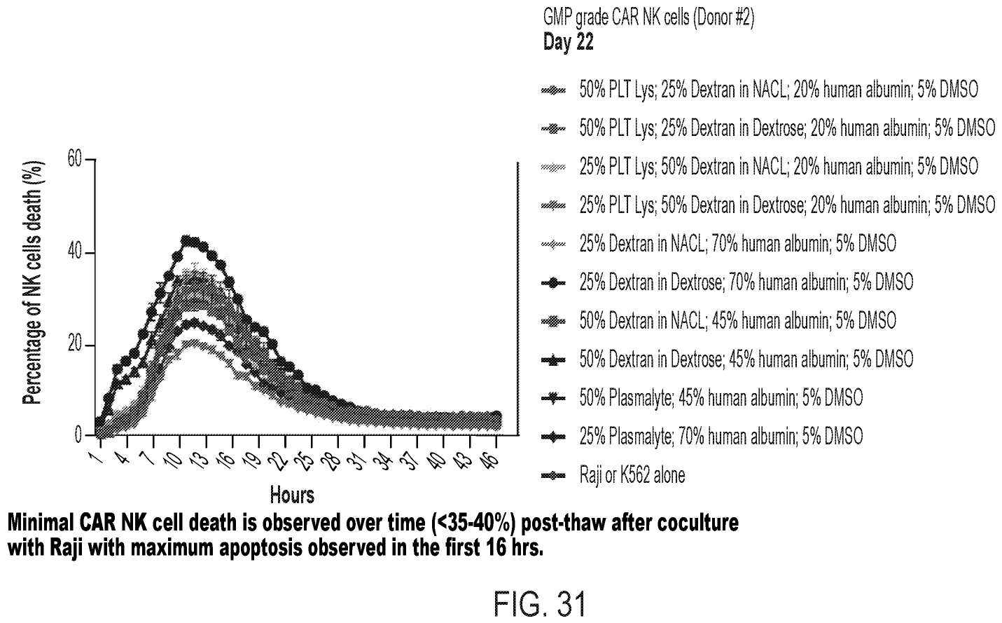

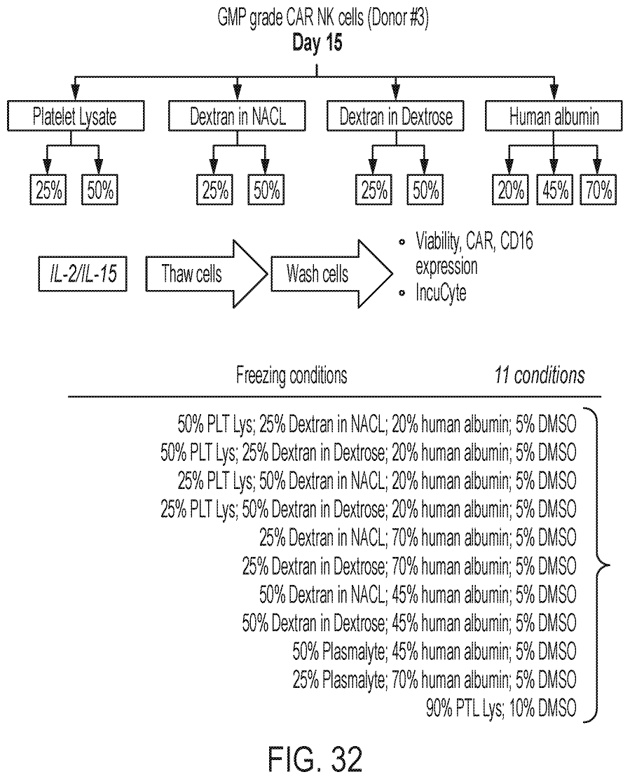

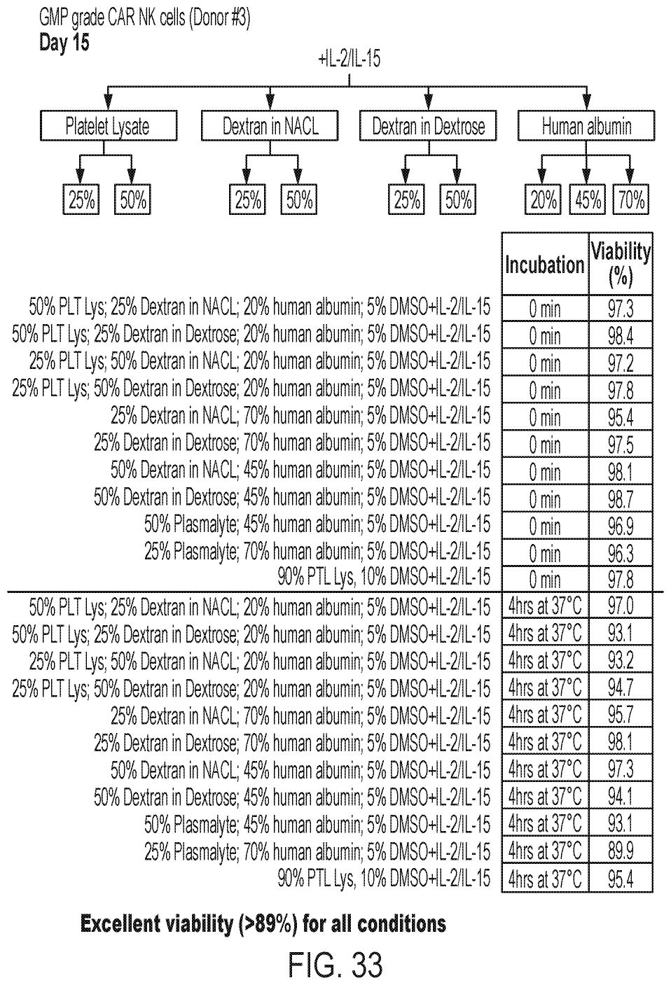

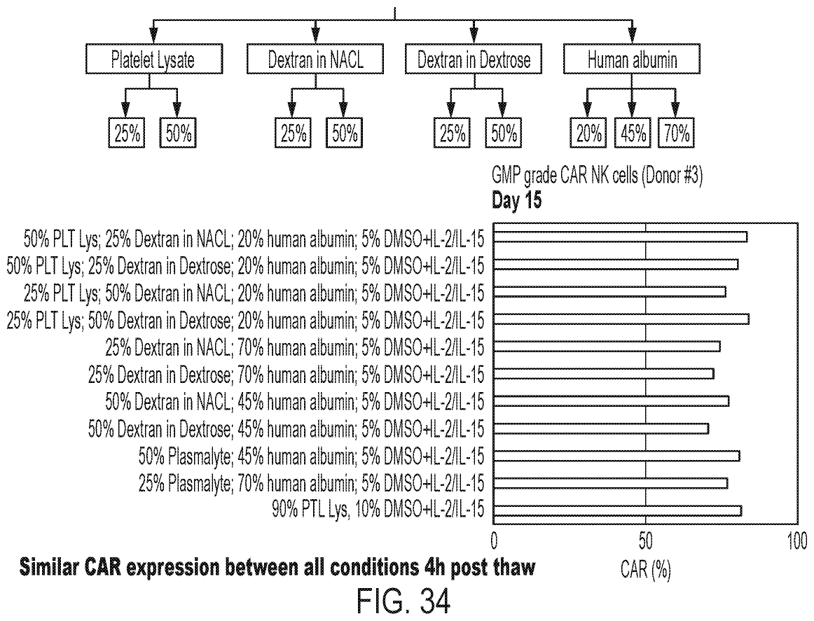

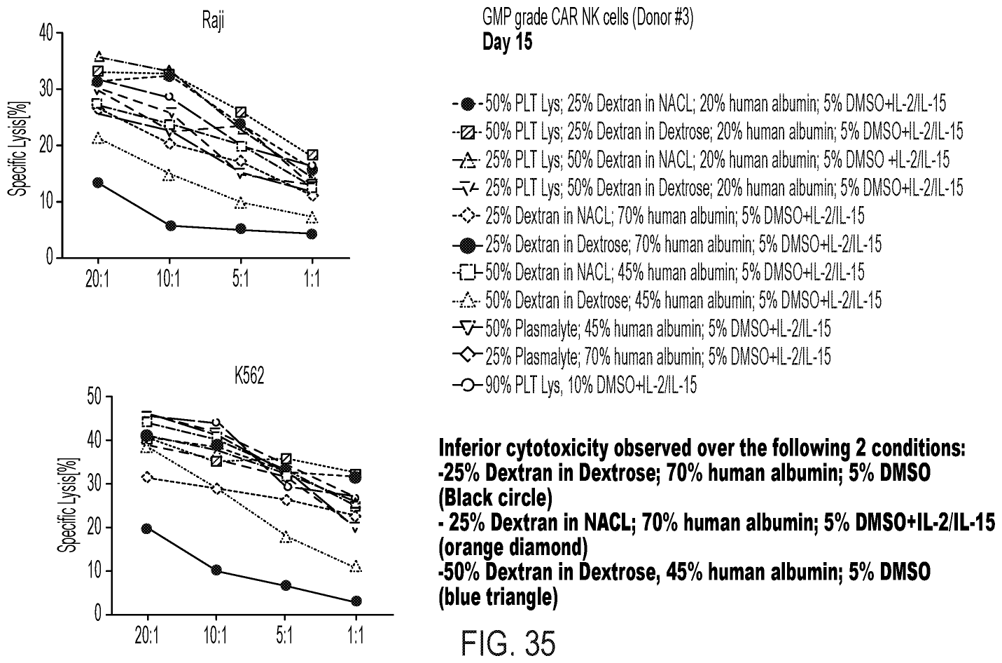

The inventors then elected to titrate components of the extracellular cryoprotectant in the freeze CAR NK cells that were expanded for 15 vs. 22 days: specifically, the concentration of PLTLysate (25% vs 50%) added to the freeze media; the concentration of dextran (25% vs 50%) and the diluent (NACL vs. dextrose); and the concentration of human albumin (20% vs 45% vs 70%). All conditions were tested with the addition of a combination of IL-2/IL-15 cytokines. FIG. 27 shows a detailed schema of these studies. FIG. 28 showed excellent viability (>87%) post thaw for all conditions tested. FIG. 29 shows that minimal CAR NK cell death (<20%) using annexin staining 4 h post-thaw most conditions tested, with the exception of freeze media containing: (i) 25% Dextran in Dextrose plus 70% human albumin plus 5% DMSO and (ii) 50% Dextran in Dextrose plus 45% human albumin plus 5% DMSO, where NK cell apoptosis post thaw was ˜30%. FIG. 30 shows excellent killing of Raji and K562 cells in the IncuCyte assay for all conditions tested. FIG. 31 shows that minimal CAR NK cell death was observed over time for most conditions except for cells frozen in 25% Dextran in Dextrose plus 70% human albumin plus 5% DMSO where >40% underwent apoptosis post-thaw after coculture with Raji, with maximum apoptosis observed in the first 16 hrs. FIG. 32 shows the schema for the next series of studies where the various freezing formulations included a combination of two cytokines (IL-2/IL-15). FIG. 33 shows excellent viability (>89%) for CAR NK cells immediate post thaw for all conditions tested. FIG. 34 shows similar CAR expression for CAR NK cells 4 h post thaw. FIG. 35 shows Inferior cytotoxicity for CAR NK cells immediate post thaw cryopreserved with the following 3 conditions:

25% Dextran in Dextrose; 70% human albumin; 5% DMSO (Black circle)

25% Dextran in NACL; 70% human albumin; 5% DMSO+IL-2/IL-15 (orange diamond)

50% Dextran in Dextrose, 45% human albumin; 5% DMSO (blue triangles)

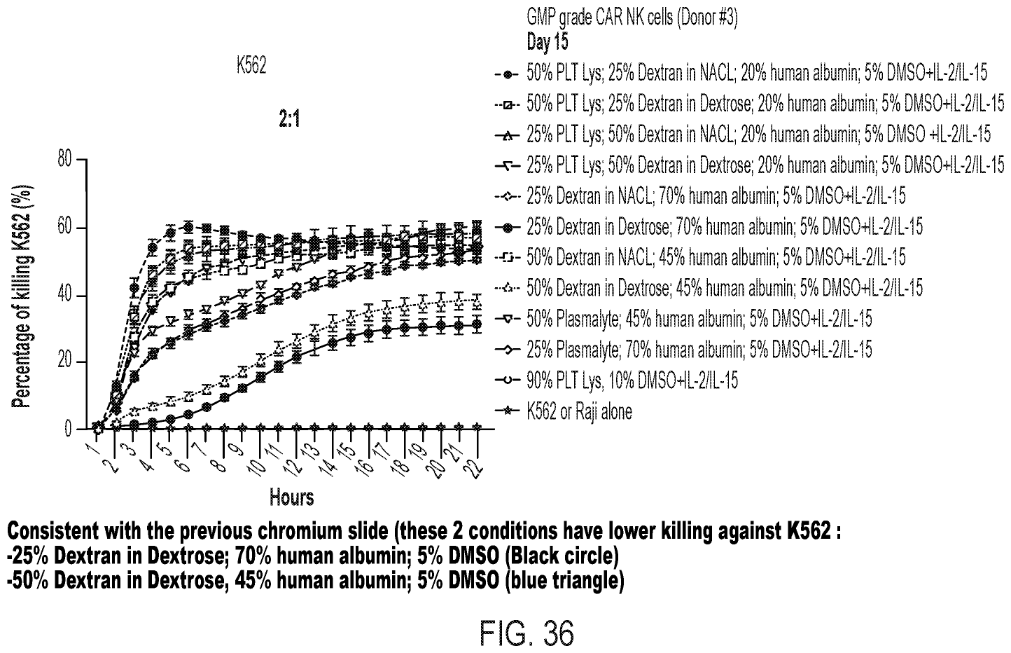

FIG. 36 in keeping with data with 51 chromium release assay in FIG. 35, live imaging using Incucyte killing assay confirmed inferior cytotoxicity against K562 targets by CAR NK cells cryopreserved with the following 2 conditions:

25% Dextran in Dextrose; 70% human albumin; 5% DMSO (Black circle)

50% Dextran in Dextrose, 45% human albumin; 5% DMSO (blue triangle)

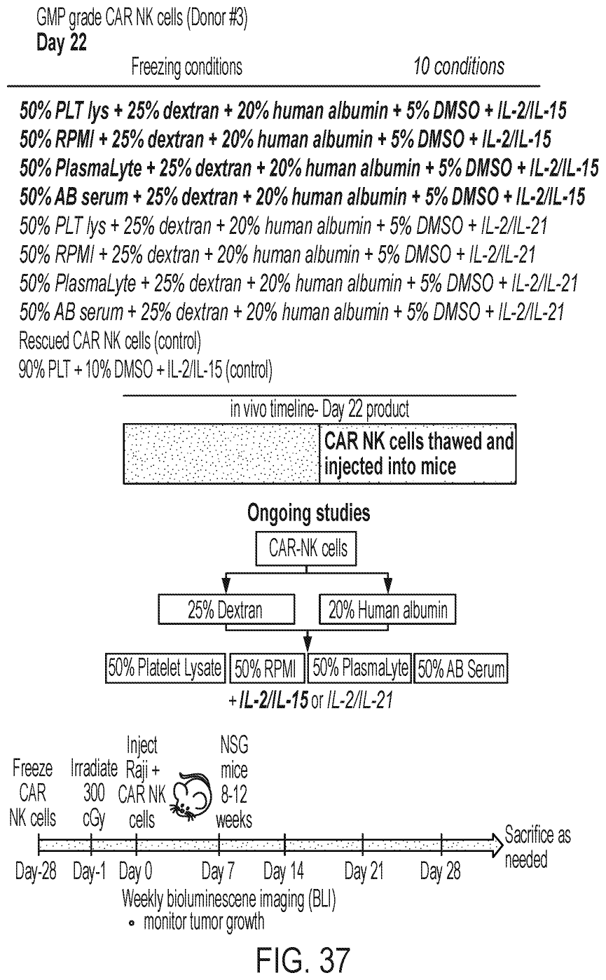

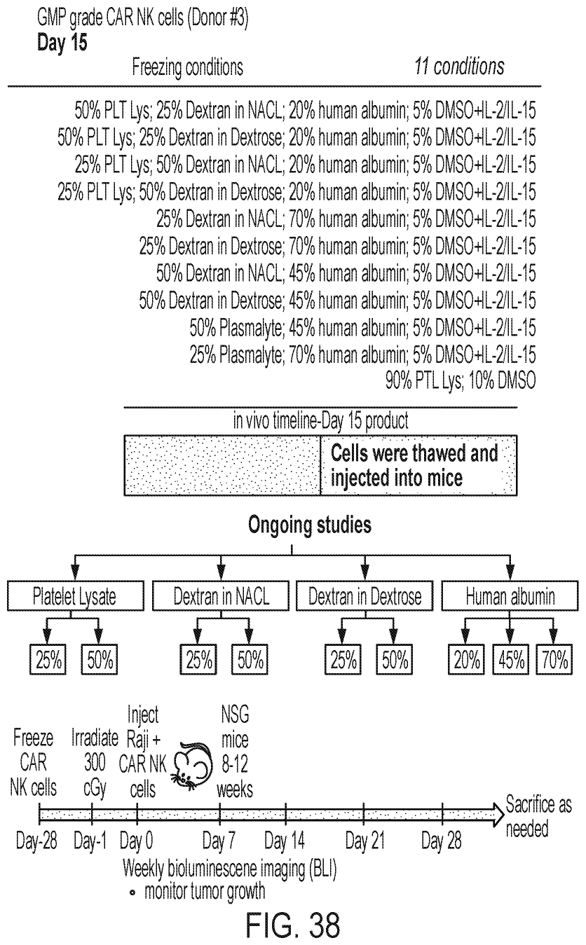

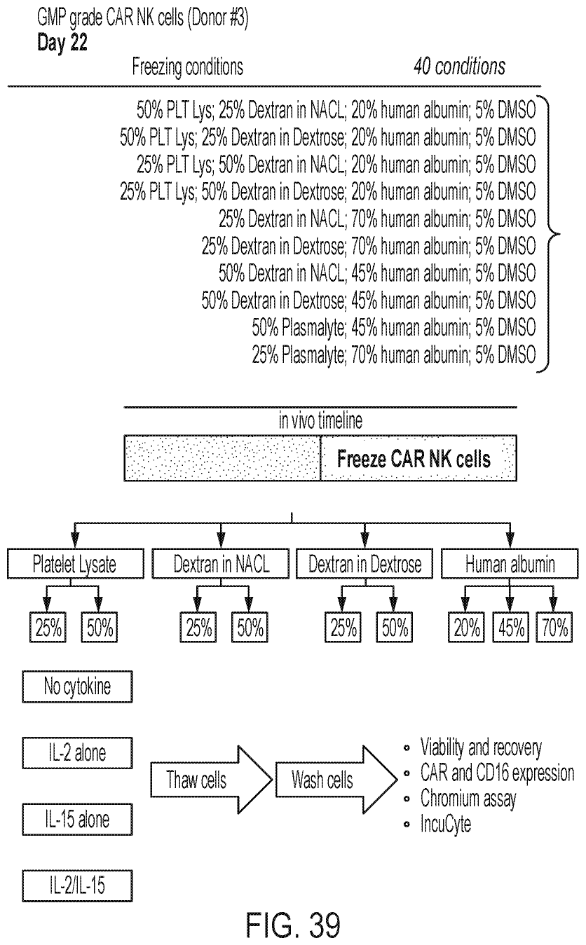

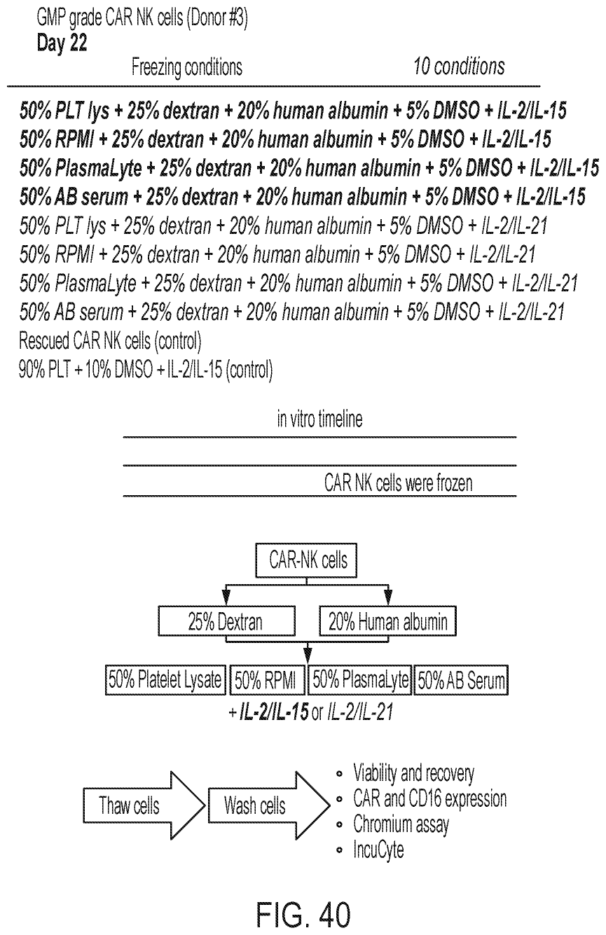

FIG. 37 through FIG. 40 show the detailed schema of subsequent studies evaluating clinical CAR-NK cell products in the various freezing formulations. These studies exhibited robust and reproducible viability and killing with the optimized formulations described above. In summary, these studies confirm that the cryopreservation formulation comprising novel concentrations of intracellular and extracellular cryoprotectants, as well as cytokines, results in a robust CAR-NK product with excellent viability, recovery and in vitro cytotoxicity following thawing.

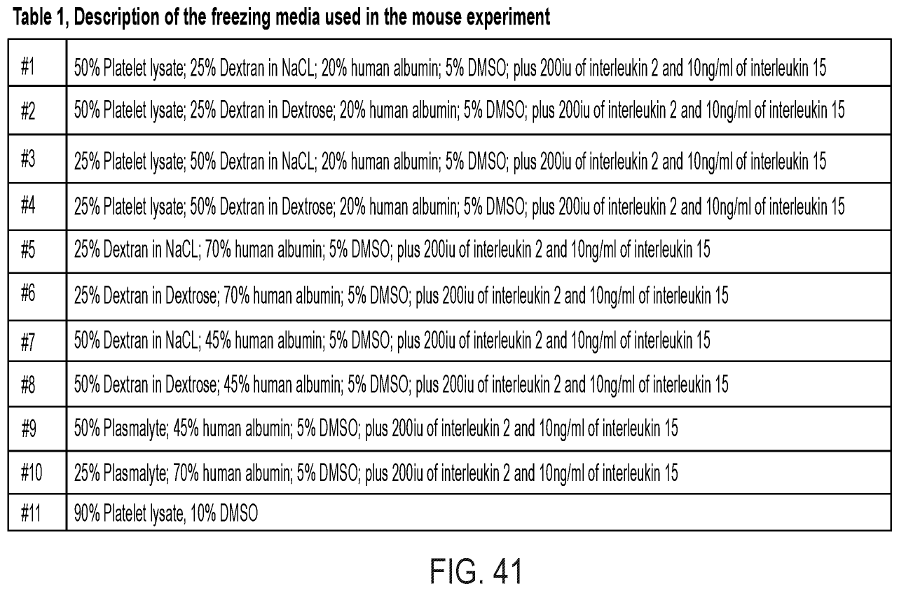

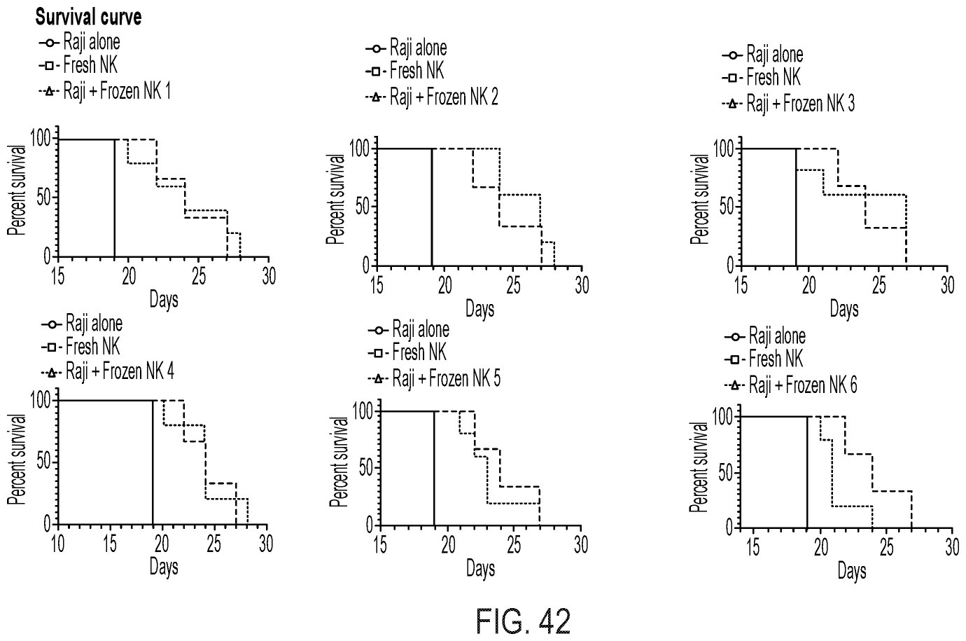

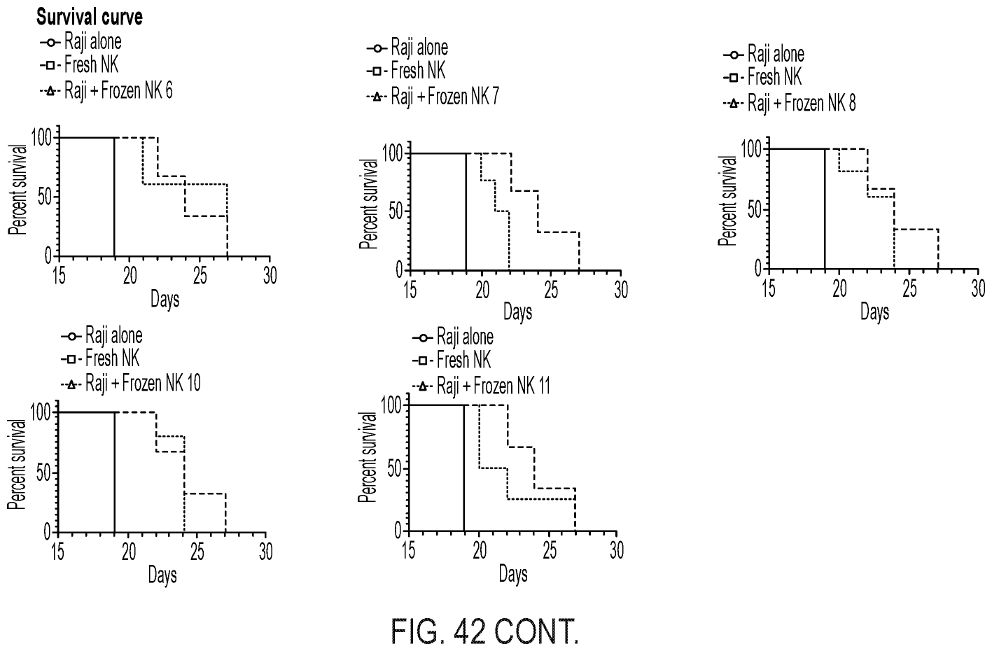

These results have been recapitulated in a xenogeneic murine model with impressive antitumor activity against Raji lymphoma cells that is comparable to that observed with fresh CAR-NK cells, as shown by bioluminescence and survival analysis (FIG. 41). Briefly, in order to identify the optimal freezing media to cryopreserve CAR NK cells, the inventors inoculated 13 cohorts of NSG mice with 2×10e4 Raji cells. On the same day (day 0), one cohort received 1×10e7 fresh CD19 CAR NK cells (positive control), 11 cohorts received 2 infusions of 1×10e7 frozen CAR NK cells (on days 0 and 7) that were cryopreserved in the freeze media as detailed in Table 1 (FIG. 41) and infused immediately post-thaw. One cohort did not received CARNK cells (negative cohort). Mice that received CAR NK cells had a statistically significant superior survival compared to mice than remained untreated (FIG. 42) irrespective of the freeze media used to cryopreserve the CAR cells, however for cohorts #6, #8 and #11, the survival was clearly inferior to the survival of mice that received fresh CAR NK cells (FIG. 42).

To further assess possible differences between fresh and frozen products, a Cox regression model for survival was utilized. Mice treated in cohorts #1 (HR=0.811, p=0.78), #2 (HR=0.6, p=0.49), #3 (HR=0.916, p=0.90), #4 (HR=0.859, p=0.83) and #7 (HR=0.883, p=0.87) had superior survival, although no statistically significant compared to mice treated with the fresh CAR NK cell product.

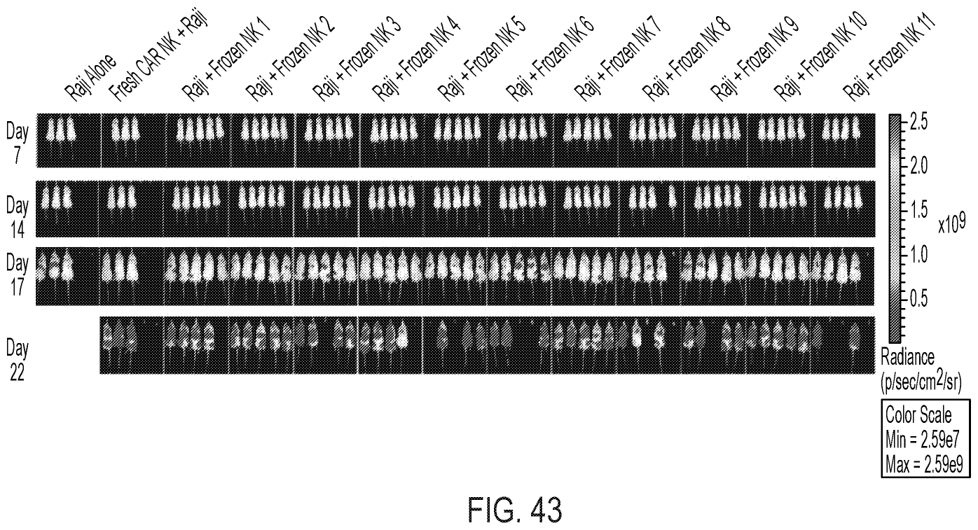

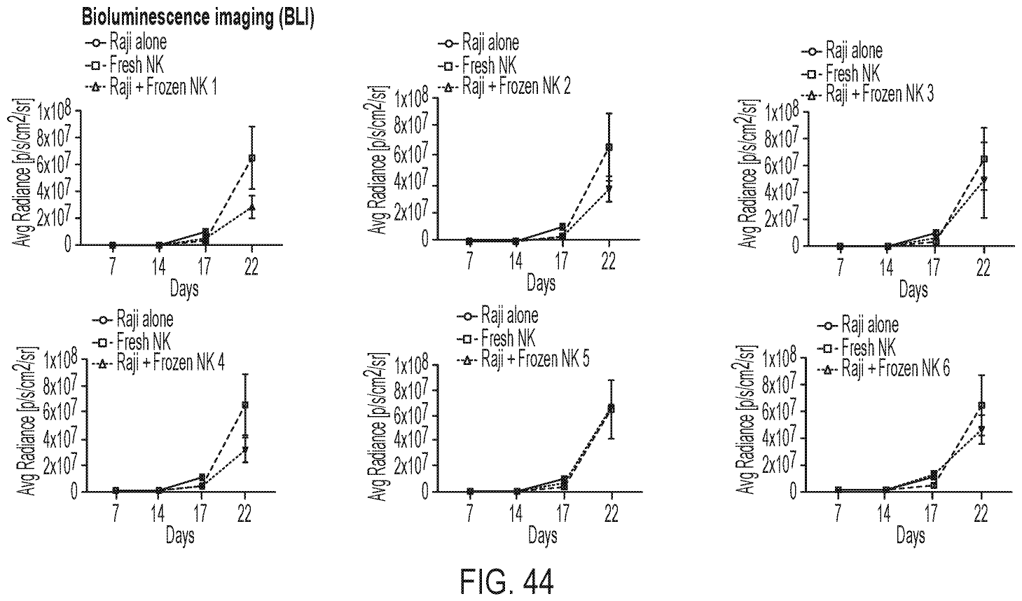

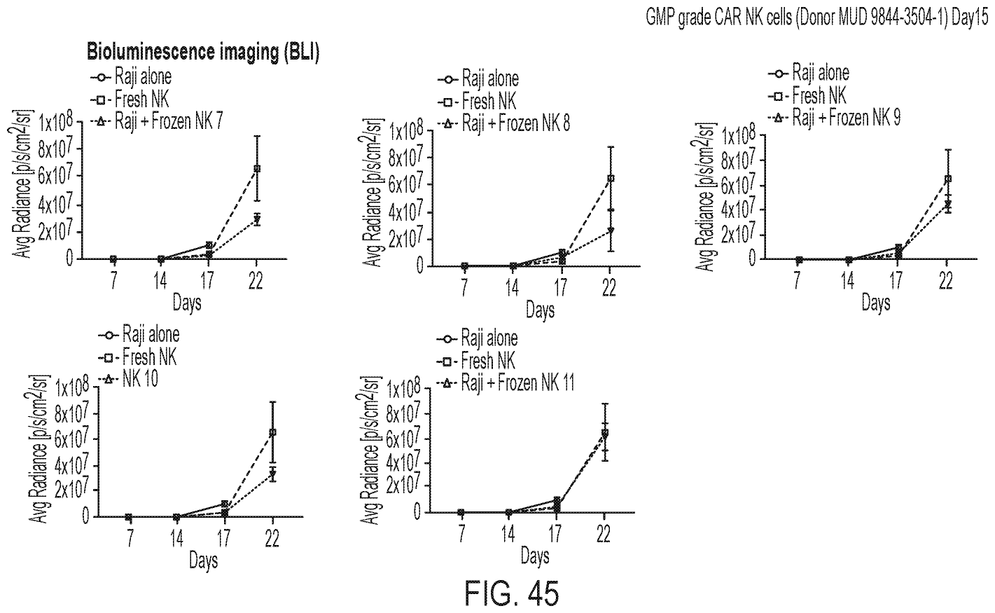

The bioluminescence imaging to determine tumor growth is presented in FIG. 43. The average radiance is presented for mice treated with CAR NK cells frozen using the different condition listed in FIG. 41, compared to mice treated with fresh CAR NK cells or no treatment as positive and negative controls, respectively. ROI is not available for animals treated with Raji alone beyond day 17 as they all succumbed to disease before the BLI scheduled for day 22. For all conditions tested, the ROI value for mice treated with frozen CAR NK cells, tumor control was either equivalent or better than that observed with fresh CAR NK group.

Utilizing methods and compositions of the disclosure, CAR-transduced cord blood derived NK cells can provide an off-the-shelf source of personalized NK cells that can recognize and attack many cancers including both liquid and solid tumors. CAR transduction or gene editing of natural killer cells from any source (cord blood, peripheral blood, bone marrow, cell lines such as NK92 cells, HSC-derived, iPSC derived) allows for longer persistence and improved efficacy of the engineered cells for use in the immunotherapy of many cancers and potentially for the treatment of many viral infections. The ability to cryopreserve NK cell such that post thaw they retain the same potency as their fresh counterpart is extremely valuable as it will allow for this type of immunotherapy to be used as an off-the-shelf therapy for patients with cancer. It is important to note that NK cells have been traditionally very difficult to freeze and there are currently no commercial or academic freezing protocols available for the cryopreservation of NK cells.

### Example 5—Specific Formulations of Cryopreservation Media

The present example provides particular formulations for cryopreservation media.

50% RPMI; 25% dextran; 20% human albumin, 5% DMSO
50% RPMI; 25% dextran; 20% human albumin, 5% DMSO + IL-2/IL-15
35% RPMI; 40% dextran; 20% human albumin, 5% DMSO
35% RPMI; 40% dextran; 20% human albumin, 5% DMSO + IL-2/IL-15
32.5% RPMI; 40% dextran; 20% human albumin, 7.5% DMSO
32.5% RPMI; 40% dextran; 20% human albumin, 7.5% DMSO + IL-2/IL-15
50% PlasmaLyte; 25% dextran; 20% human albumin, 5% DMSO
50% PlasmaLyte; 25% dextran; 20% human albumin, 5% DMSO + IL-2/IL-15
35% PlasmaLyte; 40% dextran; 20% human albumin, 5% DMSO
35% PlasmaLyte; 40% dextran; 20% human albumin, 5% DMSO + IL-2/IL-15
32.5% PlasmaLyte; 40% dextran; 20% human albumin, 7.5% DMSO
32.5% PlasmaLyte; 40% dextran; 20% human albumin, 7.5% DMSO + IL-2/IL-15
70% PlasmaLyte; 25% dextran; 5% DMSO + IL-2/IL-15
90% PLT Lys + 10% DMSO + IL-2/IL-15
50% PLT lys + 25% dextran + 20% human albumin + 5% DMSO + IL-2/IL-15
50% AB serum + 25% dextran + 20% human albumin + 5% DMSO + IL-2/IL-15
50% PLT lys + 25% dextran + 20% human albumin + 5% DMSO + IL-2/IL-21
50% RPMI + 25% dextran + 20% human albumin + 5% DMSO + IL-2/IL-21
50% PlasmaLyte + 25% dextran + 20% human albumin + 5% DMSO + IL-2/IL-21
50% AB serum + 25% dextran + 20% human albumin + 5% DMSO + IL-2/IL-21
50% PLT Lys; 25% Dextran in NACL; 20% human albumin; 5% DMSO + IL-2/IL-15
50% PLT Lys; 25% Dextran in Dextrose; 20% human albumin; 5% DMSO + IL-2/IL-15
25% PLT Lys; 50% Dextran in NACL; 20% human albumin; 5% DMSO + IL-2/IL-15
25% PLT Lys; 50% Dextran in Dextrose; 20% human albumin; 5% DMSO + IL-2/IL-15
25% Dextran in NACL; 70% human albumin; 5% DMSO + IL-2/IL-15
25% Dextran in Dextrose; 70% human albumin; 5% DMSO + IL-2/IL-15
50% Dextran in NACL; 45% human albumin; 5% DMSO + IL-2/IL-15
50% Dextran in Dextrose; 45% human albumin; 5% DMSO + IL-2/IL-15
50% Plasmalyte; 45% human albumin; 5% DMSO + IL-2/IL-15
25% Plasmalyte; 70% human albumin; 5% DMSO + IL-2/IL-15

Examples of particular formulations with certain concentrations may be utilized as follows:

50% Platelet lysate; 25% Dextran in NaCL; 20% human albumin; 5% DMSO; plus
200 iu of interleukin 2 and 10 ng/ml of interleukin 15
50% Platelet lysate; 25% Dextran in Dextrose; 20% human albumin; 5% DMSO; plus 200 iu of
interleukin 2 and 10 ng/ml of interleukin 15
25% Platelet lysate; 50% Dextran in NaCL; 20% human albumin; 5% DMSO; plus 200 iu of
interleukin 2 and 10 ng/ml of interleukin 15
25% Platelet lysate; 50% Dextran in Dextrose; 20% human albumin; 5% DMSO; plus 200 iu of
interleukin 2 and 10 ng/ml of interleukin 15
25% Dextran in NaCL; 70% human albumin; 5% DMSO; plus 200 iu of interleukin 2 and
10 ng/ml of interleukin 15
25% Dextran in Dextrose; 70% human albumin; 5% DMSO; plus 200 iu of interleukin 2 and
10 ng/ml of interleukin 15
50% Dextran in NaCL; 45% human albumin; 5% DMSO; plus 200 iu of interleukin 2 and
10 ng/ml of interleukin 15
50% Dextran in Dextrose; 45% human albumin; 5% DMSO; plus 200 iu of interleukin 2 and
10 ng/ml of interleukin 15
50% Plasmalyte; 45% human albumin; 5% DMSO; plus 200 iu of interleukin 2 and 10 ng/ml of
interleukin 15
25% Plasmalyte; 70% human albumin; 5% DMSO; plus 200 iu of interleukin 2 and 10 ng/ml of
interleukin 15
90% Platelet lysate, 10% DMSO

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A cryopreservation medium composition comprising at least one cryoprotectant, at least one serum or non-serum alternative to serum, and at least one cytokine and/or at least one growth factor.

2. The composition of claim 1, wherein the cryoprotectant is dimethyl sulfoxide (DMSO), glycerin, glycerol, hydroxyethol starch, or a combination thereof.

3. The composition of claim 1 or 2, wherein the non-serum alternative comprises platelet lysate and/or a blood product lysate or human or animal serum albumin.

4. The composition of any one of claims 1-3, wherein the at least one cytokine is a natural protein, a recombinant protein, a synthetic protein, or a mixture thereof.

5. The composition of any one of claims 1-4, wherein the at least one cytokine is a Food and Drug Administration (FDA)-approved cytokine.

6. The composition of any one of claims 1-5, wherein the composition comprises two or more cytokines.

7. The composition of any one of claims 1-6, wherein the at least one cytokine is IL-1, IL-2, IL-3, IL-4, IL-6, IL-7, IL-9, IL-10, IL-12, IL-13, IL-15, IL-17, IL-18, IL-21, IL-22, interferon, tumor necrosis factor, stem cell factor, FLT3-ligand, APRIL, thrombopoietin, erythropoietin, or a combination thereof.

8. The composition of any one of claims 1-7, wherein the serum is an animal-derived serum.

9. The composition of claim 8, wherein the animal-derived serum is human serum or bovine serum.

10. The composition of claim 9, wherein the human serum is human AB serum.

11. The composition of any one of claims 2-10, wherein the cryoprotectant comprises 4-6% of the composition.

12. The composition of any one of claims 2-10, wherein the cryoprotectant comprises 5-10% of the composition.

13. The composition of any one of claims 1-12, wherein the serum comprises 5-99% of the composition.

14. The composition of any one of claims 1-13, wherein the serum comprises 95% of the composition.

15. The composition of any one of claims 3-14, wherein the platelet lysate comprises 5%-99% of the composition.

16. The composition of any one of claims 3-15, wherein the platelet lysate comprises 95% of the composition.

17. The composition of any one of claims 7-16, wherein the IL-2 is present at a concentration of 1-5000 U/mL.

18. The composition of any one of claims 7-16, wherein the IL-2 is present at a concentration of 400 U/mL.

19. The composition of any one of claims 7-18, wherein the IL-21 is present at a concentration of 10-3000 ng/mL

20. The composition of any one of claims 7-19, wherein the IL-21 is present at a concentration of 20 ng/mL.

21. The composition of any one of claims 7-19, wherein the IL-15 is present at a concentration of 10-2000 ng/mL.

22. The composition of any one of claims 1-21, wherein the composition comprises:
(a) one or more of platelet lysate, PlasmaLyte, and Roswell Park Memorial Institute (RPMI) media;
(b) one or more of dextran, albumin, and DMSO; and
(c) one or more of IL-2, IL-15, and IL-21.

23. The composition of claim 22, wherein the platelet lysate is between 50% and 90% of the composition.

24. The composition of claim 22 or 23, wherein the platelet lysate is about 50% of the composition.

25. The composition of claim 22 or 23, wherein the platelet lysate is about 90% of the composition.

26. The composition of any one of claims 22-25, wherein the PlasmaLyte is between about 32.5% and 70% of the composition.

27. The composition of any one of claims 22-26, wherein the PlasmaLyte is about 32.5% of the composition.

28. The composition of any one of claims 22-26, wherein the PlasmaLyte is about 35% of the composition.

29. The composition of any one of claims 22-26, wherein the PlasmaLyte is about 50% of the composition.

30. The composition of any one of claims 22-26, wherein the PlasmaLyte is about 70% of the composition.

31. The composition of any one of claims 22-30, wherein the RPMI is between 32.5% and 50% of the composition.

32. The composition of any one of claims 22-31, wherein the RPMI is about 32.5% of the composition.

33. The composition of any one of claims 22-31, wherein the RPMI is about 35% of the composition.

34. The composition of any one of claims 22-31, wherein the RPMI is about 50% of the composition.

35. The composition of any one of claims 22-34, wherein the dextran is about 25-40% of the composition.

36. The composition of any one of claims 22-34, wherein the dextran is about 25% of the composition.

37. The composition of any one of claims 22-34, wherein the dextran is about 40% of the composition.

38. The composition of any one of claims 22-37, wherein the albumin is about 1-99% of the composition.

39. The composition of any one of claims 22-38, wherein the albumin is about 20% of the composition.

40. The composition of any one of claims 22-39, wherein the DMSO is about 5-7.5% of the composition.

41. The composition of any one of claims 22-40, wherein the DMSO is 5% of the composition.

42. The composition of any one of claims 22-40, wherein the DMSO is 7.5% of the composition.

43. The composition of any one of claims 1-42, wherein the composition comprises one of the following:
50% RPMI; 25% dextran; 20% human albumin, 5% DMSO
50% RPMI; 25% dextran; 20% human albumin, 5% DMSO + IL-2/IL-15
35% RPMI; 40% dextran; 20% human albumin, 5% DMSO
35% RPMI; 40% dextran; 20% human albumin, 5% DMSO + IL-2/IL-15
32.5% RPMI; 40% dextran; 20% human albumin, 7.5% DMSO
32.5% RPMI; 40% dextran; 20% human albumin, 7.5% DMSO + IL-2/IL-15
50% PlasmaLyte; 25% dextran; 20% human albumin, 5% DMSO
50% PlasmaLyte; 25% dextran; 20% human albumin, 5% DMSO + IL-2/IL-15
35% PlasmaLyte; 40% dextran; 20% human albumin, 5% DMSO
35% PlasmaLyte; 40% dextran; 20% human albumin, 5% DMSO + IL-2/IL-15
32.5% PlasmaLyte; 40% dextran; 20% human albumin, 7.5% DMSO
32.5% PlasmaLyte; 40% dextran; 20% human albumin, 7.5% DMSO + IL-2/IL-15
70% PlasmaLyte; 25% dextran; 5% DMSO + IL-2/IL-15
90% platelet lysate (PLT Lys) + 10% DMSO + IL-2/IL-15
50% PLT lys + 25% dextran + 20% human albumin + 5% DMSO + IL-2/IL-15
50% AB serum + 25% dextran + 20% human albumin + 5% DMSO + IL-2/IL-15
50% PLT lys + 25% dextran + 20% human albumin + 5% DMSO + IL-2/IL-21
50% RPMI + 25% dextran + 20% human albumin + 5% DMSO + IL-2/IL-21
50% PlasmaLyte + 25% dextran + 20% human albumin + 5% DMSO + IL-2/IL-21
50% AB serum + 25% dextran + 20% human albumin + 5% DMSO + IL-2/IL-21
50% PLT Lys; 25% Dextran in NACL; 20% human albumin; 5% DMSO + IL-2/IL-15
50% PLT Lys; 25% Dextran in Dextrose; 20% human albumin; 5% DMSO + IL-2/IL-15
25% PLT Lys; 50% Dextran in NACL; 20% human albumin; 5% DMSO + IL-2/IL-15
25% PLT Lys; 50% Dextran in Dextrose; 20% human albumin; 5% DMSO + IL-2/IL-15
25% Dextran in NACL; 70% human albumin; 5% DMSO + IL-2/IL-15
25% Dextran in Dextrose; 70% human albumin; 5% DMSO + IL-2/IL-15
50% Dextran in NACL; 45% human albumin; 5% DMSO + IL-2/IL-15
50% Dextran in Dextrose; 45% human albumin; 5% DMSO + IL-2/IL-15
50% Plasmalyte; 45% human albumin; 5% DMSO + IL-2/IL-15
25% Plasmalyte; 70% human albumin; 5% DMSO + IL-2/IL-15

44. The composition of any one of claims 1-43, wherein the composition comprises one of the following:
50% Platelet lysate; 25% Dextran in NaCL; 20% human albumin; 5% DMSO; plus 200
International Units (iu) of interleukin 2 and 10 ng/ml of interleukin 15
50% Platelet lysate; 25% Dextran in Dextrose; 20% human albumin; 5% DMSO; plus 200 iu of
interleukin 2 and 10 ng/ml of interleukin 15
25% Platelet lysate; 50% Dextran in NaCL; 20% human albumin; 5% DMSO; plus 200 iu of
interleukin 2 and 10 ng/ml of interleukin 15
25% Platelet lysate; 50% Dextran in Dextrose; 20% human albumin; 5% DMSO; plus 200 iu of
interleukin 2 and 10 ng/ml of interleukin 15
25% Dextran in NaCL; 70% human albumin; 5% DMSO; plus 200 iu of interleukin 2 and
10 ng/ml of interleukin 15
25% Dextran in Dextrose; 70% human albumin; 5% DMSO; plus 200 iu of interleukin 2 and
10 ng/ml of interleukin 15
50% Dextran in NaCL; 45% human albumin; 5% DMSO; plus 200 iu of interleukin 2 and
10 ng/ml of interleukin 15
50% Dextran in Dextrose; 45% human albumin; 5% DMSO; plus 200 iu of interleukin 2 and
10 ng/ml of interleukin 15
50% Plasmalyte; 45% human albumin; 5% DMSO; plus 200 iu of interleukin 2 and 10 ng/ml of
interleukin 15
25% Plasmalyte; 70% human albumin; 5% DMSO; plus 200 iu of interleukin 2 and 10 ng/ml of
interleukin 15
90% Platelet lysate, 10% DMSO

45. The composition of any one of claims 1-44, further comprising a plurality of cells.

46. The composition of claim 44, wherein the cells are NK cells, T cells, B cells, iNKT cells, gamma-delta T cells, MSCs, macrophages, monocytes, dendritic cells, NKT cells derived from mature cells, tumor cells, stem cells, induced pluripotent stem cells, MSCs, or a mixture thereof.

47. The composition of claim 46, wherein the NK cells are expanded NK cells.

48. A pharmaceutical composition comprising the composition of any one of claims 22-47 and a pharmaceutically acceptable carrier.

49. A method of producing the composition of any one of claims 22-47, comprising the step of subjecting the cells to an effective amount of the cryopreservation medium composition.

50. The method of claim 49, wherein the cells are immune cells or stem cells.

51. The method of claim 49 or 50, wherein the cells are NK cells, T cells, NKT cells, invariant NKT cells, B cells, MSCs, monocytes, macrophages, dendritic cells derived from mature cells, tumor cells, stem cells, induced pluripotent stem cells, or hematopoietic stem cells.

52. The method of any one of claims 49-51, wherein the cells are expanded NK cells.

53. A population of cells produced according to the method of any one of claims 49-53.

54. The population of claim 53, and a pharmaceutically acceptable carrier.

55. The population of claim 53 or 53, wherein the cells are immune cells or stem cells.

56. The population of claim 53, 54, or 55, wherein the cells are NK cells, T cells, NKT cells, B cells, invariant NKT cells derived from mature cells, tumor cells, stem cells, induced pluripotent stem cells, or MSCs.

57. The population of claim 56, wherein the NK cells are expanded NK cells.

58. A method of treating an immune-related disorder in a subject comprising administering an effective amount of a thawed population of any one of claims 53-57 to the subject.

59. The method of claim 58, wherein the immune-related disorder is a cancer, autoimmune disorder, graft versus host disease, allograft rejection, or an inflammatory condition.

60. The method of claim 58 or 59, wherein the population comprises cells that are NK cells, T cells, invariant NKT cells, B cells, NKT cells, monocytes, macrophages, dendritic cells derived from mature cells, tumor cells, stem cells, induced pluripotent stem cells, or MSCs.

61. The method of any one of claims 58-60, wherein the immune-related disorder is cancer.

62. The method of any one of claims 58-61, wherein the at least one cytokine is present in the composition at a level that provides no therapeutic effect to the subject.

63. The method of any one of claims 58-62, wherein the cells in the composition are washed prior to the administering step.

64. The method of any one of claims 58-62, wherein the cells in the composition are not washed prior to the administering step.

65. A method of preserving cells that are sensitive to cryopreservation, comprising the step of subjecting cells that are sensitive to cryopreservation to an effective amount of the cryopreservation medium composition of any one of claims 1-47.

66. The method of claim 65, wherein the cells are NK cells, T cells, NKT cells, B cells, invariant NKT cells, monocytes, macrophages, dendritic cells derived from mature cells, stem cells, induced pluripotent stem cells, or MSCs.

67. The method of claim 65 or 66, further comprising the step of obtaining or providing the cells to be subjected to the cryopreservation medium composition.

68. The method of any one of claims 65-67, wherein following cryopreservation and thawing of the cells, an effective amount of the cells are delivered to a subject in need thereof.

69. The method of claim 68, wherein the cells are allogeneic or autologous with respect to the subject.

70. The method of claim 68 or 69, wherein the subject has cancer, autoimmune disorder, graft versus host disease, allograft rejection, or an inflammatory condition.

71. The method of any one of claims 68-70, wherein the at least one cytokine is present in the composition at a level that provides no therapeutic effect to the subject.

72. The method of any one of claims 68-71, wherein the cells in the composition are washed prior to the administering step.

73. The method of any one of claims 68-71, wherein the cells in the composition are not washed prior to the administering step.

74. A method of maintaining the viability of a population of cells over at least 50% percent following cryopreservation of the population, comprising the step of subjecting the population to an effective amount of the cryopreservation medium composition of any one of claims 1-44 and thawing said population, wherein upon thawing the viability of the population is over at least 50%.

75. The method of claim 74, wherein upon thawing the viability of the population of cells is over at least 55, 60, 65, 70, 75, 80, 85, 90, 91, 92, 93, 94, 95, 96, 97, 98, or 99% following cryopreservation of the population.

76. The method of any one of claims 74-75, wherein the cells are NK cells, T cells, NKT cells, B cells, invariant NKT cells derived from mature cells, tumor cells, stem cells, induced pluripotent stem cells, monocytes, macrophages, dendritic cells, or MSCs.

77. A method of prolonging the shelf life of a population of cells upon cryopreservation of the population, comprising the step of subjecting the population to an effective amount of the cryopreservation medium composition of any one of claims 1-44.

78. The method of claim 77, wherein the cells are NK cells, T cells, NKT cells, B cells, invariant NKT cells derived from mature cells, tumor cells, stem cells, induced pluripotent stem cells, monocytes, macrophages, dendritic cells, or MSCs.

79. The method of claim 77 or 78, further comprising the step of obtaining the cells.

80. The method of any one of claims 77-79, wherein following cryopreservation and thawing of the cells, an effective amount of the cells are delivered to a subject in need thereof.

81. The method of claim 80, wherein the cells are allogeneic or autologous with respect to the subject.

82. The method of claim 80 or 81, wherein the subject has cancer, autoimmune disorder, graft versus host disease, allograft rejection, a bacterial, viral or fungal infection, or an inflammatory condition.

83. The method of any one of claims 80-82, wherein the at least one cytokine is present in the composition at a level that provides no therapeutic effect to the subject.

84. The method of any one of claims 80-83, wherein the cells in the composition are washed prior to the administering step.

85. The method of any one of claims 80-83, wherein the cells in the composition are not washed prior to the administering step.

86. A method of thawing a population cells that have been cryopreserved with the cryopreservation medium composition of any one of claims 1-44, comprising the steps of:
exposing the population of cells to an effective amount of the cryopreservation medium composition to produce a cryopreserved population; and
exposing the cryopreserved population to suitable thawing conditions.

87. A method of delivering cells to a target site or tissue in an individual, comprising the step of infusing an effective amount of cells to the target site or tissue substantially immediately or directly following thawing of the cells, wherein the cells were cryopreserved in the cryopreservation medium composition of any one of claims 1-44.

88. The method of claim 87, wherein the target site or tissue is cancerous.

89. The method of claim 87, wherein the target site or tissue is a solid tumor.

90. The method of any one of claims 87-89, wherein at least one cytokine is present in the composition at a level that provides no therapeutic effect to the subject.

91. The method of any one of claims 87-90, wherein the cells in the composition are washed prior to the administering step.

92. One or more immune cells, comprised in the cryopreservation medium of any one of claims 1-44.

93. The cell or cells of claim 92, wherein the cell or cells are NK cells, T cells, invariant NKT cells, B cells, NKT cells, monocytes, macrophages, dendritic cells derived from mature cells, stem cells, induced pluripotent stem cells, MSCs, or a mixture thereof.


SEQUENCE LISTING (6 sequences)

SEQ ID NO: 1 - DNA, 18 bp, Artificial Sequence
  Description of Artificial Sequence Synthetic oligonucleotide
tgctgttgaggagctgga

SEQ ID NO: 2 - DNA, 18 bp, Artificial Sequence
  Description of Artificial Sequence Synthetic oligonucleotide
agcacaccaggcagagtt

SEQ ID NO: 3 - DNA, 18 bp, Artificial Sequence
  Description of Artificial Sequence Synthetic oligonucleotide
cggctgaggagcggaaga

SEQ ID NO: 4 - DNA, 18 bp, Artificial Sequence
  Description of Artificial Sequence Synthetic oligonucleotide
tggaggtgagcaatcccc

SEQ ID NO: 5 - DNA, 20 bp, Artificial Sequence
  Description of Artificial Sequence Synthetic oligonucleotide
ttaatacgactcactatagg

SEQ ID NO: 6 - DNA, 20 bp, Artificial Sequence
  Description of Artificial Sequence Synthetic oligonucleotide
gttttagagctagaaatagc